(19) United States
(12) Patent Application Publication
(10) Pub. No.: US 2024/0315128 A1
(43) Pub. Date: Sep. 19, 2024

(54) COMPOUND CONTAINING CARBAZOLE DERIVATIVE AND ORGANIC ELECTROLUMINESCENT DEVICE COMPRISING SAME

(71) Applicant: Shijiazhuang Chengzhi Yonghua Display Material Co., Ltd., Shijiazhuang (CN)

(72) Inventors: Zaifeng XIE, Shijiazhuang (CN); Tianyu WEI, Shijiazhuang (CN); Songyuan LIU, Shijiazhuang (CN)

(73) Assignee: Shijiazhuang Chengzhi Yonghua Display Material Co., Ltd., Shijiazhuang (CN)

(21) Appl. No.: 18/668,318

(22) Filed: May 20, 2024

(30) Foreign Application Priority Data
May 29, 2023 (CN) 202310619306.4

(51) Int. Cl. H10K 85/60; C07B 59/00; H10K 50/15

(52) U.S. Cl. CPC H10K 85/633; C07B 59/004; H10K 50/15; H10K 85/615; H10K 85/636; H10K 85/654; H10K 85/6572; H10K 85/6574; H10K 85/6576; H10K 85/658; C07B 2200/05

(57) ABSTRACT

The present invention discloses a compound containing a carbazole derivative and an organic electroluminescent device comprising the compound. The structure of the compound containing the carbazole derivative is one of the following structures. The compound has a relatively high hole transport rate, a relatively high HOMO energy level value, and a relatively good thermal stability. When the compound containing the carbazole derivative as provided by the present invention is applied to a hole transport layer of an organic electroluminescent device, the hole transport layer can improve the luminous efficiency of the organic electroluminescent device, reduce the driving voltage of the organic electroluminescent device, prolong the lifetime of the organic electroluminescent device, and overcome the defects in the prior art.

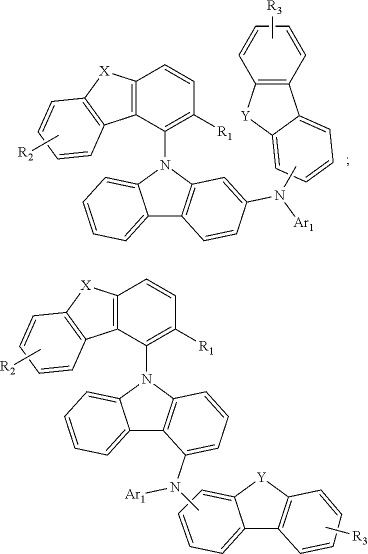

### TECHNICAL FIELD

The present invention belongs to the technical field of organic light-emitting semiconductors and particularly includes a compound containing a carbazole derivative and an organic electroluminescent device comprising the compound.

### BACKGROUND ART

An organic electroluminescent device generally comprises an anode, a cathode, and an organic layer between the two electrodes, wherein the organic layer can be composed of a hole injection layer, a hole transport layer, a luminescent layer (containing a host material and a dopant material), an electron transport layer, an electron injection layer, etc. The light-emitting process of the organic electroluminescent device can be simply divided into four steps, i.e., carrier injection, carrier transport, exciton generation by carrier recombination, and exciton radiation transition. (1) Carrier injection: in the OLED device, when a voltage is applied to the electrodes, an electric field is generated, and at this time, holes are generated at the anode, which holes overcome the barrier potential between the anode and the hole injection layer and are injected into the HOMO energy level of the adjacent hole injection layer. Similarly, under the action of the electric field, the electrons generated at the cathode overcome the barrier potential and are then injected into the LUMO energy level of the electron injection layer. (2) Carrier transport: After the carriers are injected from the electrodes, the holes in the anode migrate from the HOMO levels of the hole injection layer and the hole transport layer to the luminescent layer under the action of the electric field, and similarly, electron migration occurs from the LUMO level of the organic material. When the electric field intensity is constant, the mobilities of holes and electrons in organic thin film materials are definite, so the transport rates of holes or electrons in the device are mainly determined depending on the mobility. (3) Exciton generation by carrier recombination: Under the action of the electric field, holes and electrons are transferred to the HOMO and LUMO energy levels of the luminescent layer, respectively, and they recombine in the luminescent material to form excitons. (4) Exciton light radiation attenuation process: After the electrons and holes in the luminescent layer recombine to form excitons, they are in a higher energy state, i.e., an excited state. At this time, the excitons release energy and returns to the ground state (S₀ state) by radiation transition or non-radiation transition, and the excitons in the excited state are accompanied by light emission when radiation transition occurs.

The hole transport material has great influence on the performance of the organic light-emitting device. For devices that works continuously, the operating half-life of the hole transport material is related to the barrier potential of hole injection, i.e., to the difference between the ionization potential of the hole transport material and the work function of an ITO film. When the difference between the work function of the ITO film and the ionization potential of the hole transport material is relatively large, a higher barrier potential will be introduced at the interface between the ITO film and the hole transport layer. In addition, the basic function of adding the hole transport layer in the organic electroluminescent device is to improve the transport rate of holes and effectively block electrons in the luminescent layer, so as to realize the maximum recombination between carriers and improve the luminous efficiency. Therefore, it is required that the hole transport material should have a relatively good electron-donating property, a relatively high hole mobility, a relatively high HOMO energy level value, and a relatively good thermal stability.

However, none of currently existing hole transport layer materials can meet the requirements of organic electroluminescent devices in terms of hole transport rate, HOMO energy level and thermal stability, so it is urgent to develop a novel organic material for organic electroluminescent devices.

### SUMMARY OF THE INVENTION

In view of this, the present invention provides a compound containing a carbazole derivative and an organic electroluminescent device containing the compound. The compound containing the carbazole derivative not only has a relatively high hole transport rate and a relatively high HOMO energy level value but also has a relatively good thermal stability. When the compound containing the carbazole derivative as provided by the present invention is applied to a hole transport layer of an organic electroluminescent device, the hole transport layer can improve the luminous efficiency of the organic electroluminescent device, reduce the driving voltage of the organic electroluminescent device, prolong the lifetime of the organic electroluminescent device, and overcome the defects in the prior art.

In order to achieve the above object of the present invention, the following technical solution is used in the present invention. In a first aspect, the present invention provides a compound containing a carbazole derivative, wherein the general structural formula of the compound containing the carbazole derivative is one of the following structures:

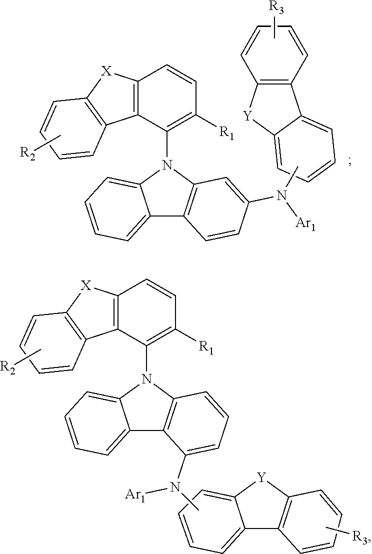

wherein
R₁ is selected from any one of hydrogen, deuterated or non-deuterated phenyl, and deuterated or non-deuterated alkyl with a carbon atom number of 1 to 5;
R₂ represents hydrogen on a benzene ring connected thereto, wherein any hydrogen can be independently replaced by deuterium;
R₃ represents hydrogen on a benzene ring connected thereto, wherein any hydrogen can be independently replaced by any one of deuterium, deuterated or non-deuterated phenyl, deuterated or non-deuterated alkyl with a carbon atom number of 1 to 5, and deuterated or non-deuterated alkenyl with a carbon atom number of 2 to 5;
X and Y are each independently selected from any one of non-bonding,

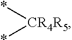

O, and S, and X and Y are not both selected from non-bonding;
Ar₁ is selected from any one of or a combination of arbitrarily some of substituted or unsubstituted aryl with a carbon atom number of 6 to 30, substituted or unsubstituted fused cyclic aryl with a carbon atom number of 10 to 30, and substituted or unsubstituted fused cyclic heteroaryl with a carbon atom number of 9 to 30;
R₄ and R₅ are each independently selected from any one of or a combination of arbitrarily some of hydrogen, deuterium, substituted or unsubstituted alkyl with a carbon atom number of 1 to 10, substituted or unsubstituted cycloalkyl with a carbon atom number of 3 to 15, and substituted or unsubstituted aryl with a carbon atom number of 6 to 15;
when R₄, R₅, and Ar₁ have substituents, the substituents on R₄, R₅, and Ar₁ are each independently selected from any one of or a combination of arbitrarily some of deuterium, alkyl with a carbon atom number of 1 to 10, aryl with a carbon atom number of 6 to 30, heteroaryl with a carbon atom number of 5 to 30, fused cyclic aryl with a carbon atom number of 10 to 30, fused cyclic heteroaryl with a carbon atom number of 9 to 30, and cycloalkyl with a carbon atom number of 3 to 10; and the above two or more adjacent substituents can be bonded to one another via a linker group or a single bond to form an aliphatic ring with a carbon atom number of 6 to 15 or an aromatic ring with a carbon atom number of 6 to 30; and any hydrogen on the compound containing the carbazole derivative can be independently replaced by deuterium, alkyl, or cycloalkyl.

In conjunction with the first aspect, the general structural formula of the compound containing the carbazole derivative is selected from any one of the following structures:

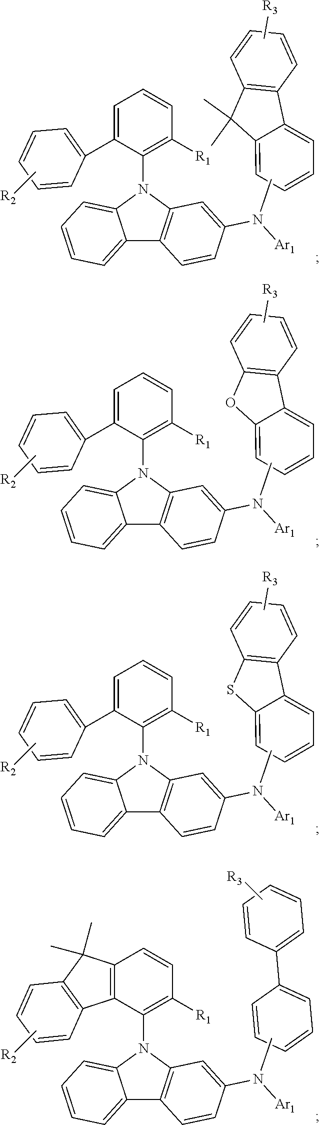

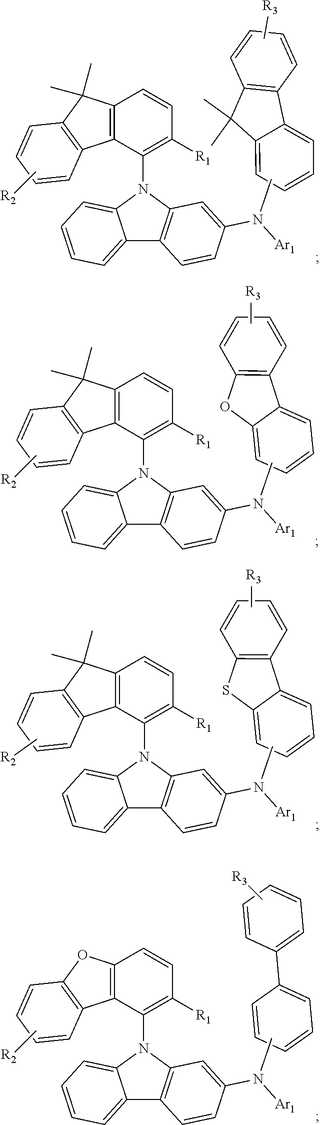

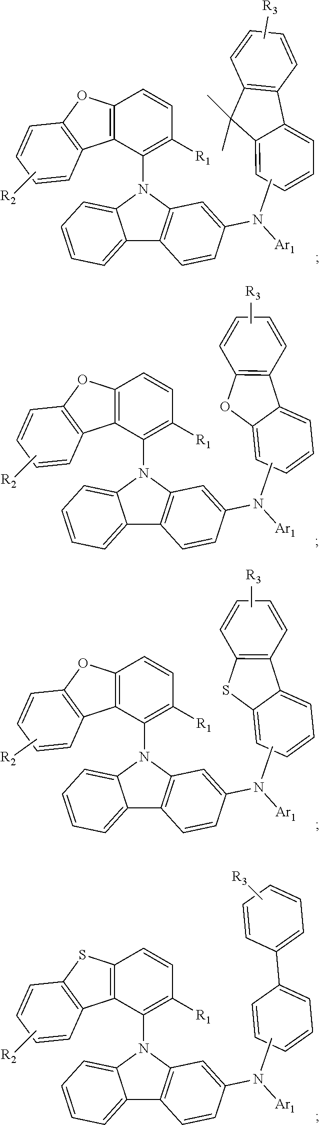

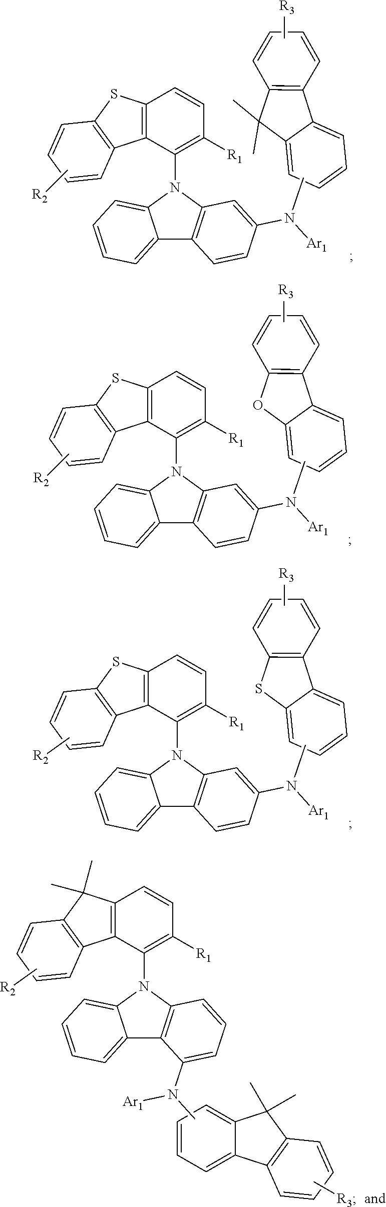

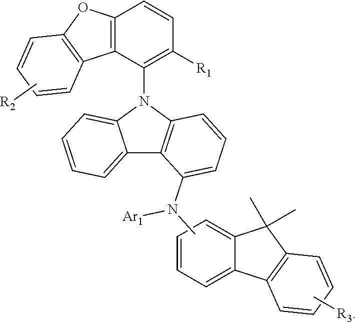

In conjunction with the first aspect, the general structural formula of the compound containing the carbazole derivative is selected from any one of the following structures:

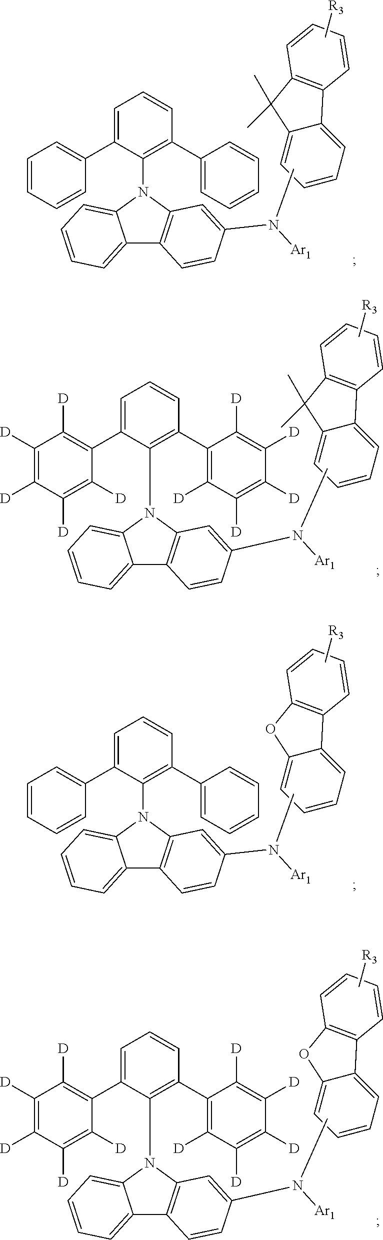

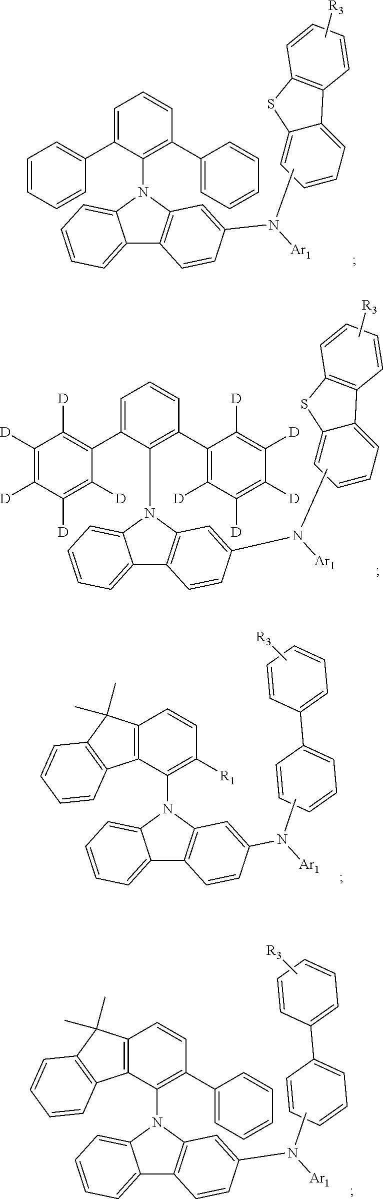

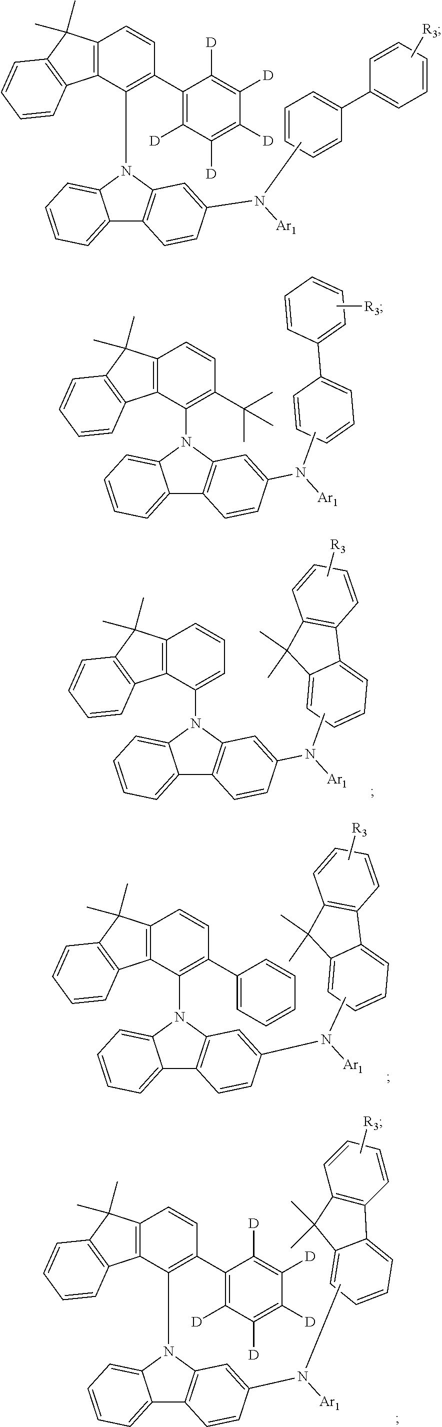

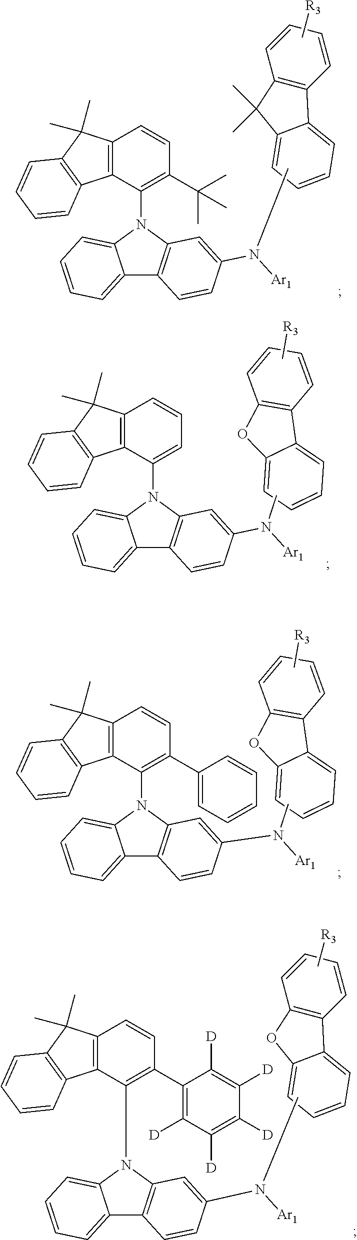

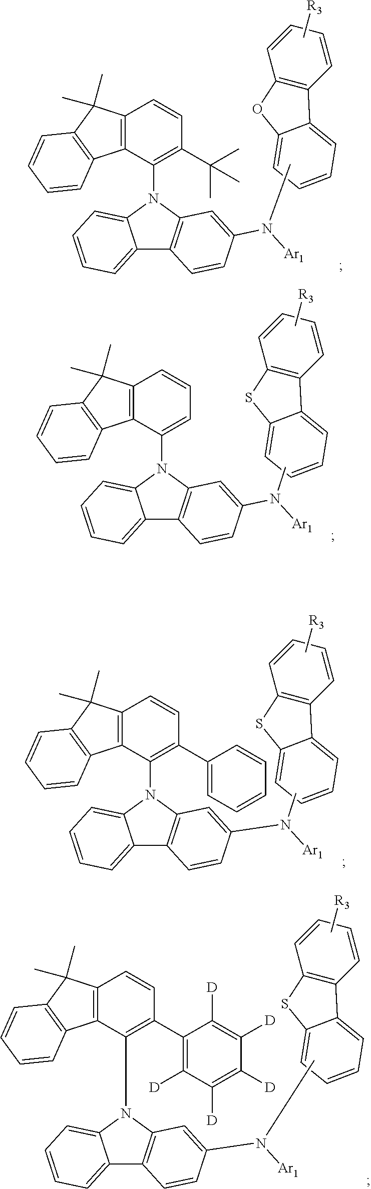

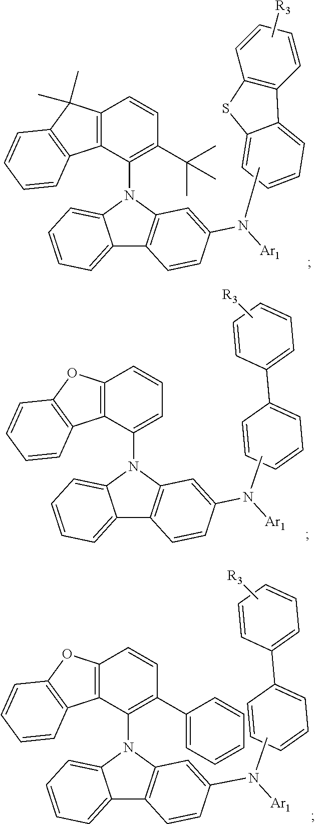

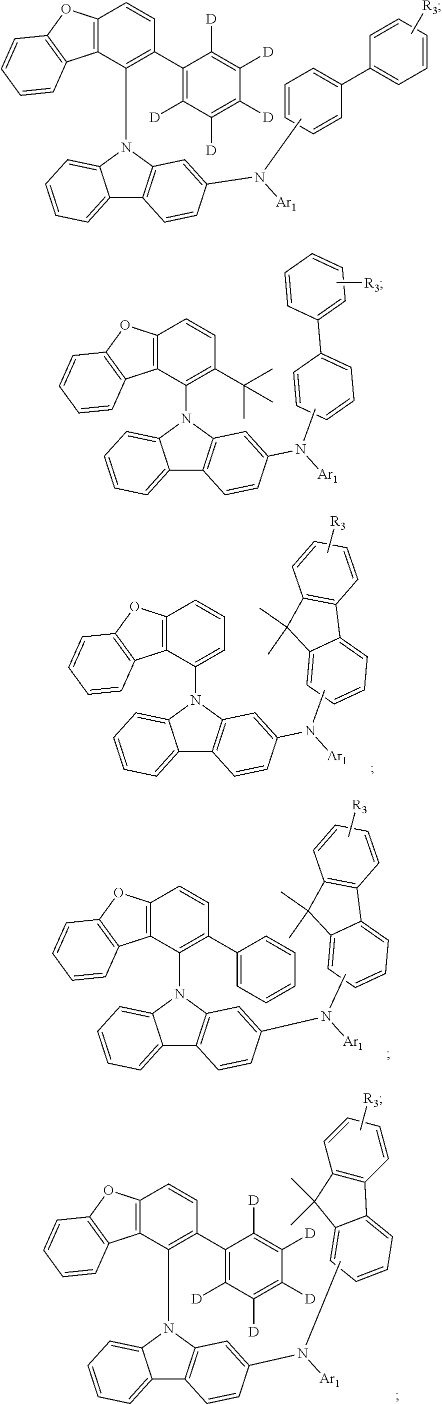

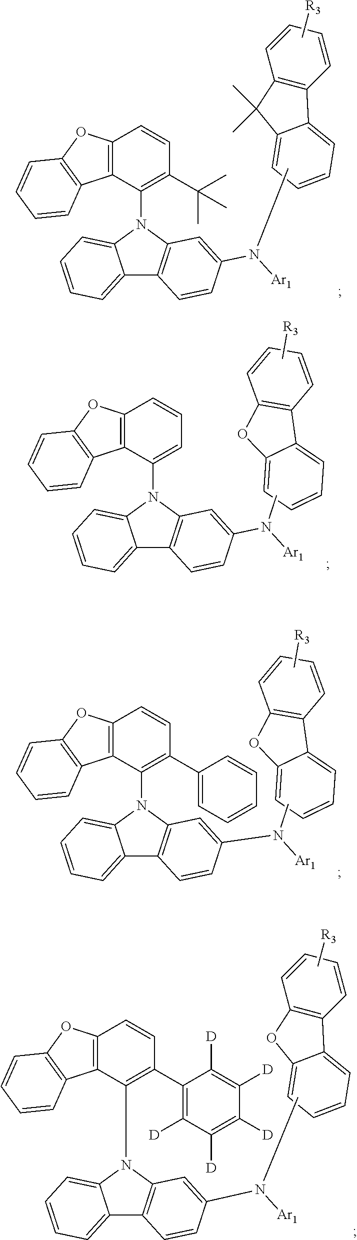

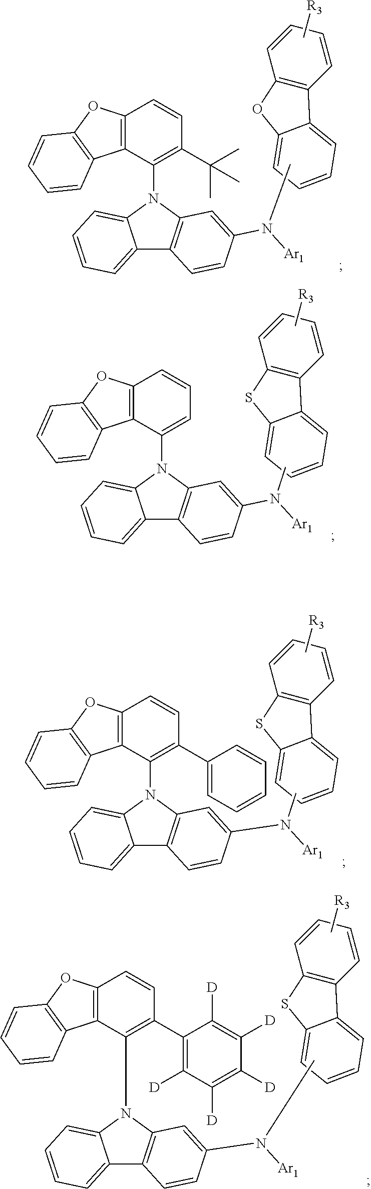

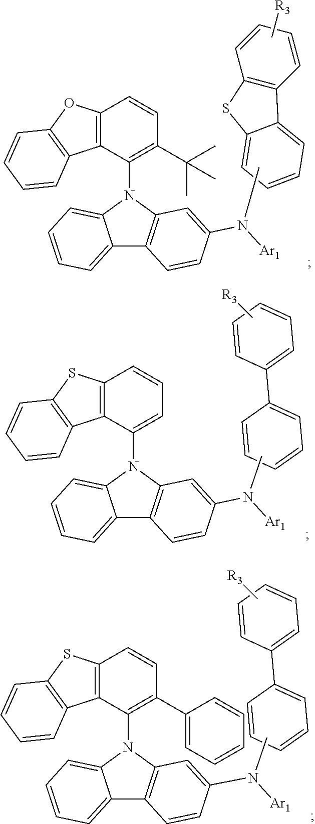

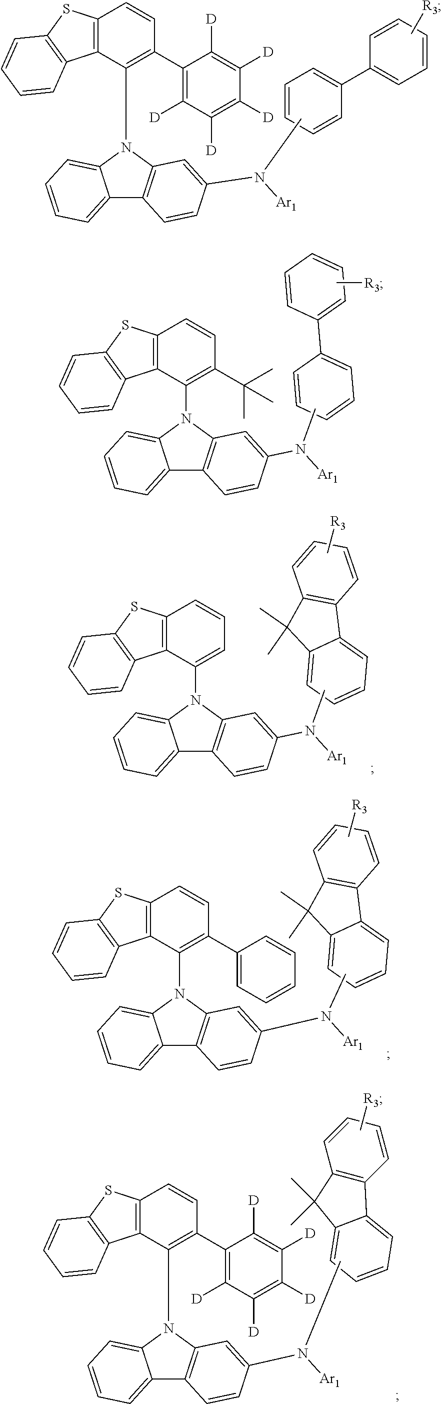

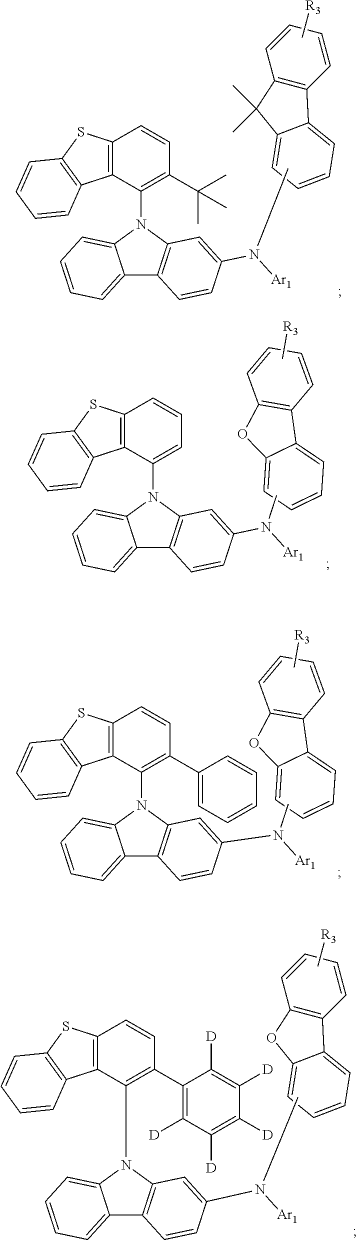

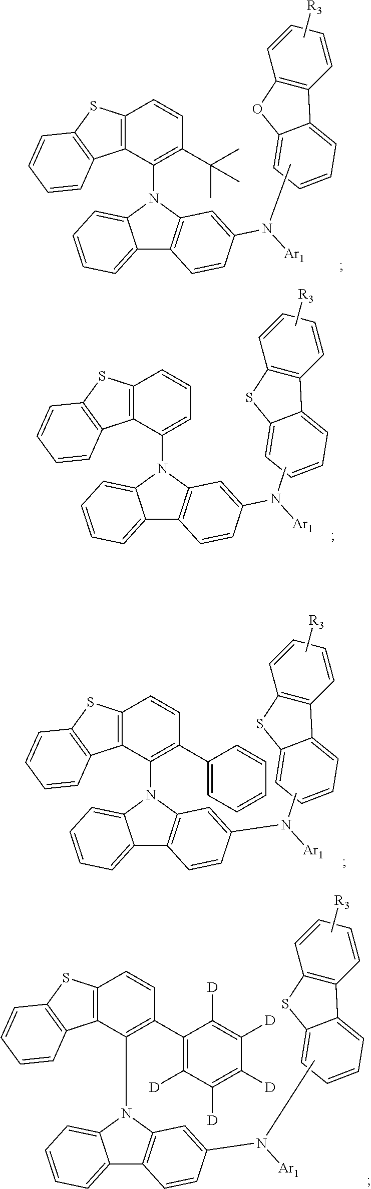

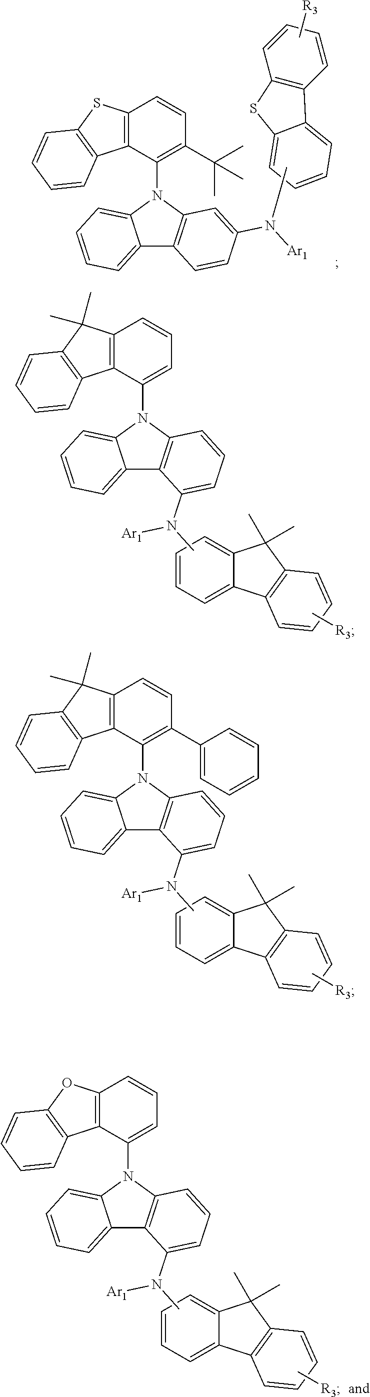

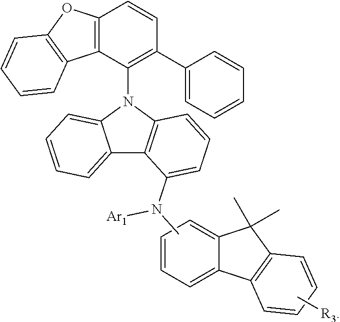

In conjunction with the first aspect, the general structural formula of the compound containing the carbazole derivative is selected from any one of the following structures:

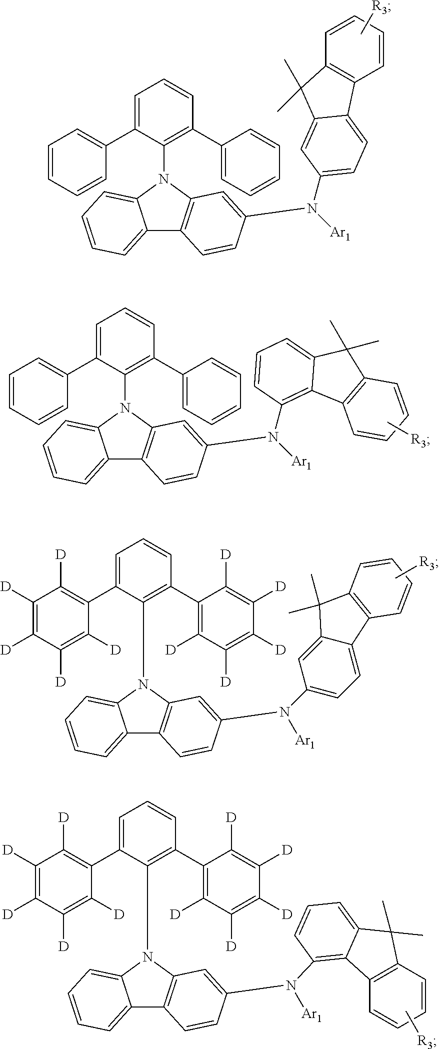

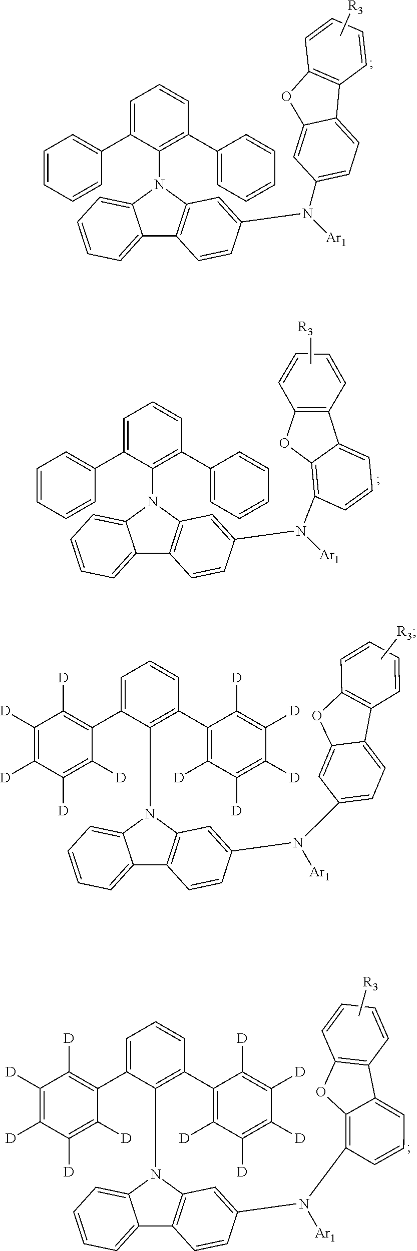

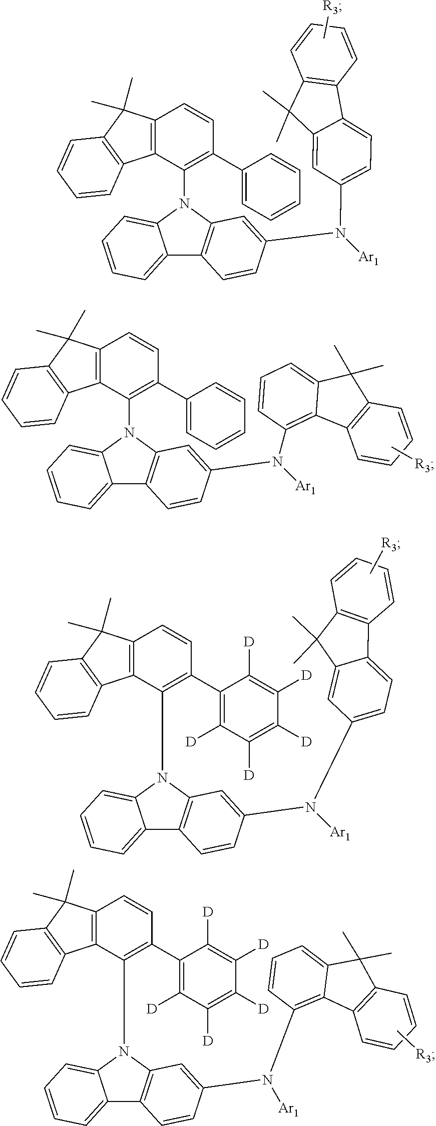

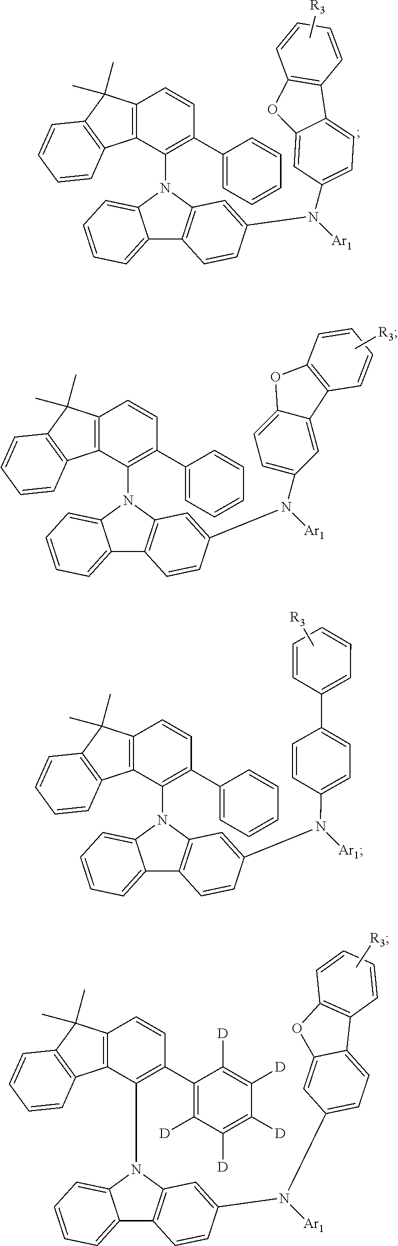

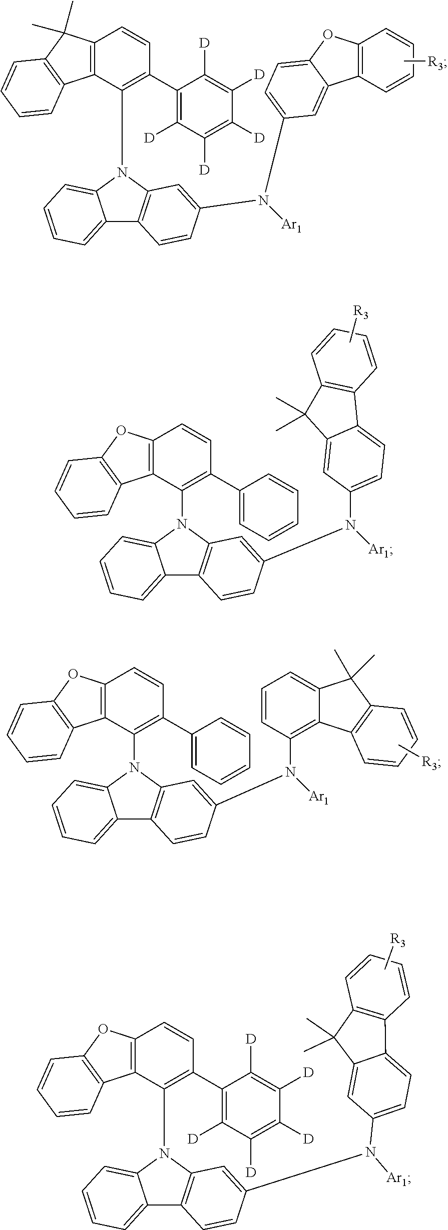

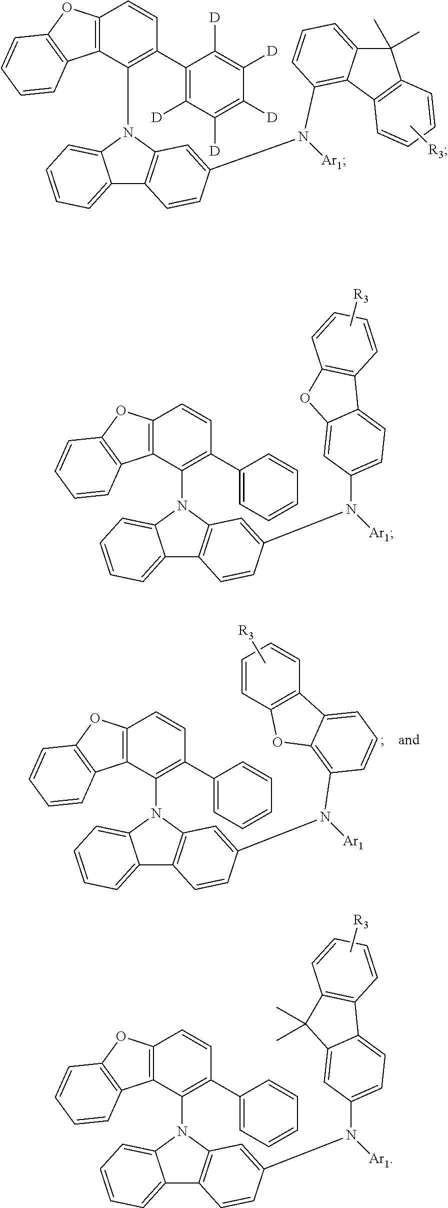

In conjunction with the first aspect, Ar₁ is selected from any one of substituted or unsubstituted aryl with a carbon atom number of 6 to 20, substituted or unsubstituted naphthyl, substituted or unsubstituted phenanthryl, substituted or unsubstituted 9,9-dimethylfluorenyl, and deuterated or non-deuterated phenyldibenzofuranyl.

In conjunction with the first aspect, the compound containing the carbazole derivative is selected from any one of the following compounds:

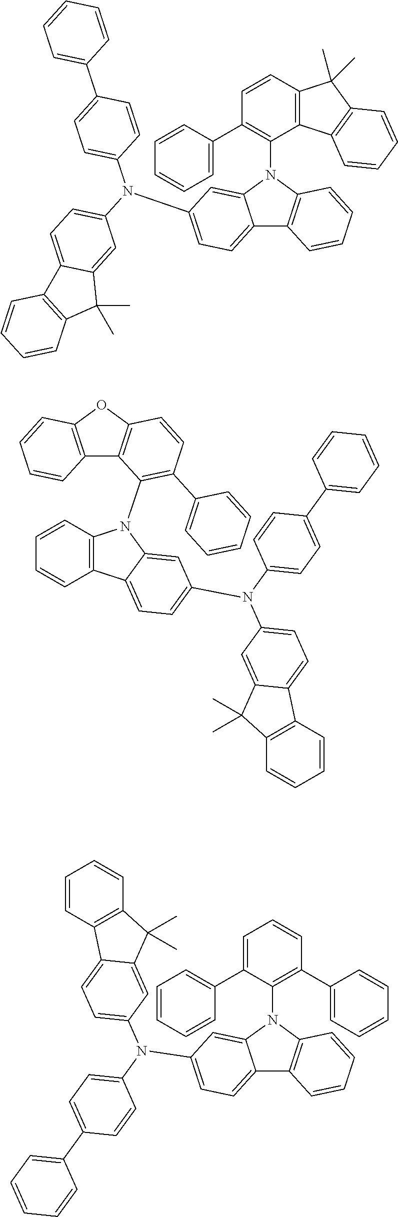 [SMILES: CC1(C)c2ccccc2-c2ccc(N(c3ccc(-c4ccccc4)cc3)c3ccc4c5ccccc5n(-c5c(-c6ccccc6)ccc6c5-c5ccccc5C6(C)C)c4c3)cc21 ; CC1(C)c2ccccc2-c2ccc(N(c3ccc(-c4ccccc4)cc3)c3ccc4c5ccccc5n(-c5c(-c6ccccc6)ccc6oc7ccccc7c56)c4c3)cc21 ; CC1(C)c2ccccc2-c2ccc(N(c3ccc(-c4ccccc4)cc3)c3ccc4c5ccccc5n(-c5c(-c6ccccc6)cccc5-c5ccccc5)c4c3)cc21]

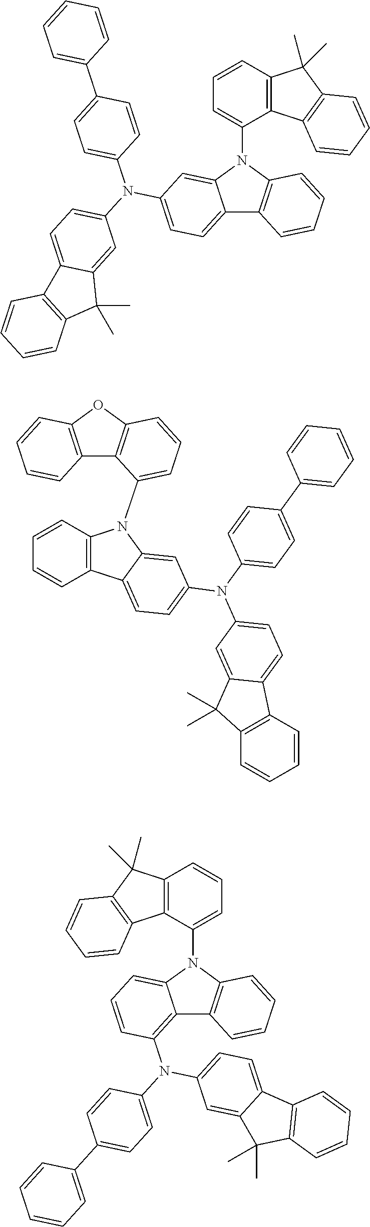 [SMILES: CC1(C)c2ccccc2-c2ccc(N(c3ccc(-c4ccccc4)cc3)c3ccc4c5ccccc5n(-c5cccc6c5-c5ccccc5C6(C)C)c4c3)cc21 ; CC1(C)c2ccccc2-c2ccc(N(c3ccc(-c4ccccc4)cc3)c3ccc4c5ccccc5n(-c5cccc6oc7ccccc7c56)c4c3)cc21 ; CC1(C)c2ccccc2-c2ccc(N(c3ccc(-c4ccccc4)cc3)c3cccc4c3c3ccccc3n4-c3cccc4c3-c3ccccc3C4(C)C)cc21]

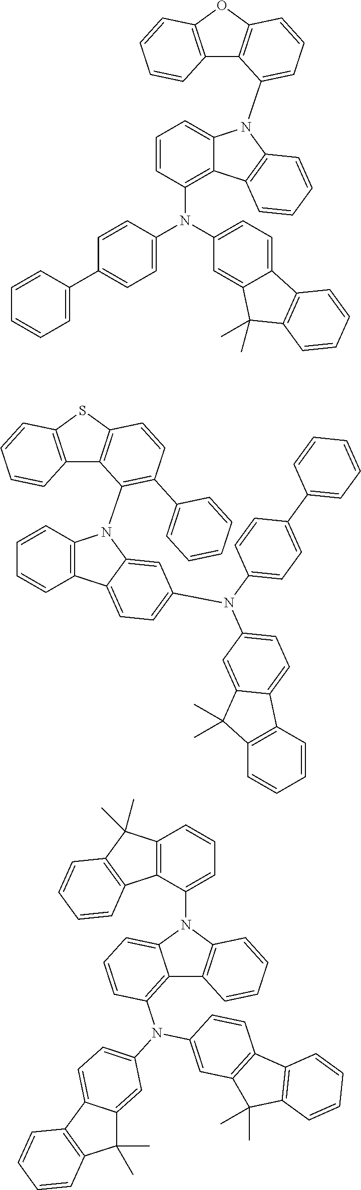 [SMILES: CC1(C)c2ccccc2-c2ccc(N(c3ccc(-c4ccccc4)cc3)c3cccc4c3c3ccccc3n4-c3cccc4oc5ccccc5c34)cc21 ; CC1(C)c2ccccc2-c2ccc(N(c3ccc(-c4ccccc4)cc3)c3ccc4c5ccccc5n(-c5c(-c6ccccc6)ccc6sc7ccccc7c56)c4c3)cc21 ; CC1(C)c2ccccc2-c2ccc(N(c3ccc4c(c3)C(C)(C)c3ccccc3-4)c3cccc4c3c3ccccc3n4-c3cccc4c3-c3ccccc3C4(C)C)cc21]

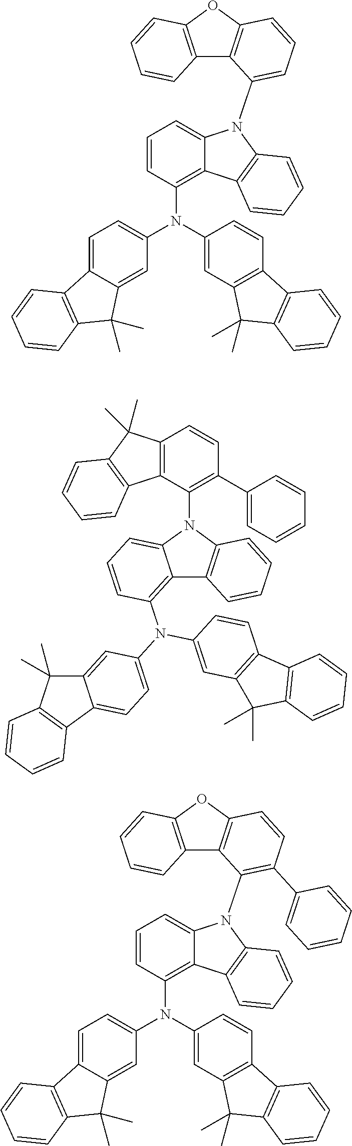 [SMILES: CC1(C)c2ccccc2-c2ccc(N(c3ccc4c(c3)C(C)(C)c3ccccc3-4)c3cccc4c3c3ccccc3n4-c3cccc4oc5ccccc5c34)cc21 ; CC1(C)c2ccccc2-c2ccc(N(c3ccc4c(c3)C(C)(C)c3ccccc3-4)c3cccc4c3c3ccccc3n4-c3c(-c4ccccc4)ccc4c3-c3ccccc3C4(C)C)cc21 ; CC1(C)c2ccccc2-c2ccc(N(c3ccc4c(c3)C(C)(C)c3ccccc3-4)c3cccc4c3c3ccccc3n4-c3c(-c4ccccc4)ccc4oc5ccccc5c34)cc21]

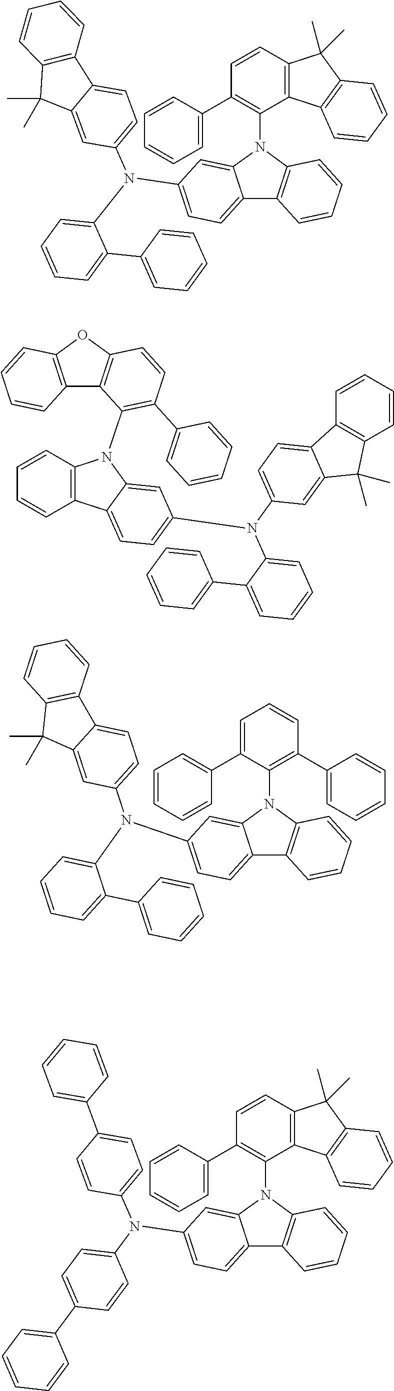 [SMILES: CC1(C)c2ccccc2-c2ccc(N(c3ccc4c5ccccc5n(-c5c(-c6ccccc6)ccc6c5-c5ccccc5C6(C)C)c4c3)c3ccccc3-c3ccccc3)cc21 ; CC1(C)c2ccccc2-c2ccc(N(c3ccc4c5ccccc5n(-c5c(-c6ccccc6)ccc6oc7ccccc7c56)c4c3)c3ccccc3-c3ccccc3)cc21 ; CC1(C)c2ccccc2-c2ccc(N(c3ccc4c5ccccc5n(-c5c(-c6ccccc6)cccc5-c5ccccc5)c4c3)c3ccccc3-c3ccccc3)cc21 ; CC1(C)c2ccccc2-c2c1ccc(-c1ccccc1)c2-n1c2ccccc2c2ccc(N(c3ccc(-c4ccccc4)cc3)c3ccc(-c4ccccc4)cc3)cc21]

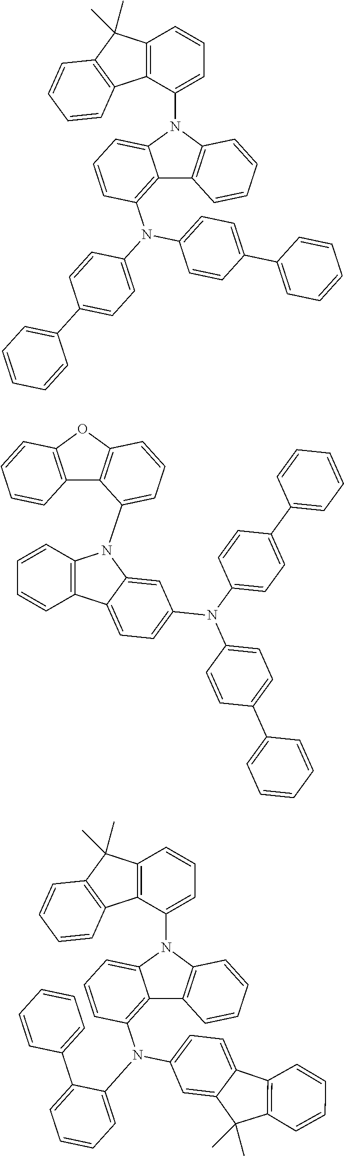 [SMILES: CC1(C)c2ccccc2-c2c(-n3c4ccccc4c4c(N(c5ccc(-c6ccccc6)cc5)c5ccc(-c6ccccc6)cc5)cccc43)cccc21 ; c1ccc(-c2ccc(N(c3ccc(-c4ccccc4)cc3)c3ccc4c5ccccc5n(-c5cccc6oc7ccccc7c56)c4c3)cc2)cc1 ; CC1(C)c2ccccc2-c2ccc(N(c3ccccc3-c3ccccc3)c3cccc4c3c3ccccc3n4-c3cccc4c3-c3ccccc3C4(C)C)cc21]

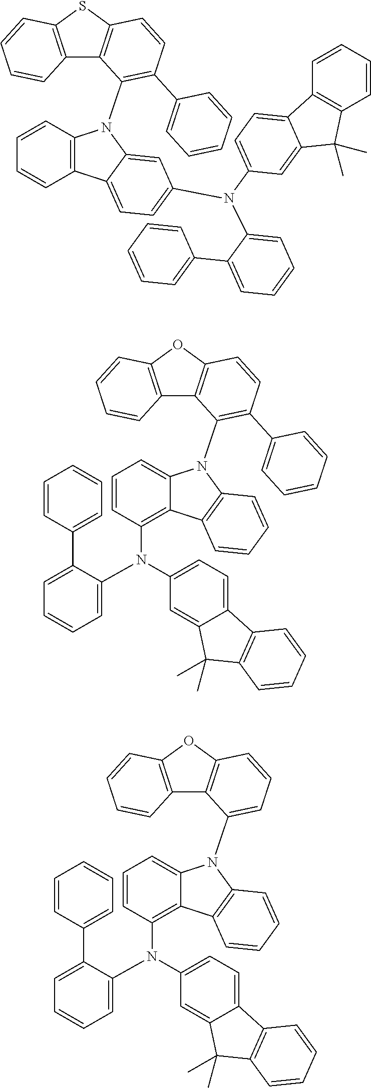 [SMILES: CC1(C)c2ccccc2-c2ccc(N(c3ccc4c5ccccc5n(-c5c(-c6ccccc6)ccc6sc7ccccc7c56)c4c3)c3ccccc3-c3ccccc3)cc21 ; CC1(C)c2ccccc2-c2ccc(N(c3ccccc3-c3ccccc3)c3cccc4c3c3ccccc3n4-c3c(-c4ccccc4)ccc4oc5ccccc5c34)cc21 ; CC1(C)c2ccccc2-c2ccc(N(c3ccccc3-c3ccccc3)c3cccc4c3c3ccccc3n4-c3cccc4oc5ccccc5c34)cc21]

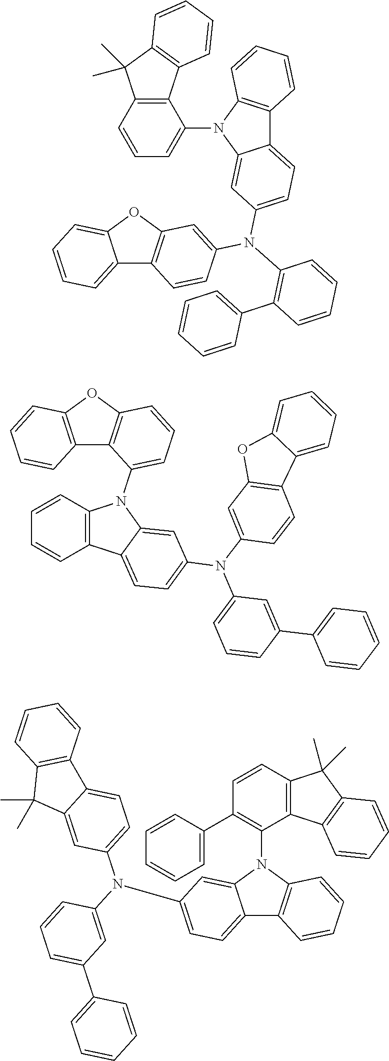

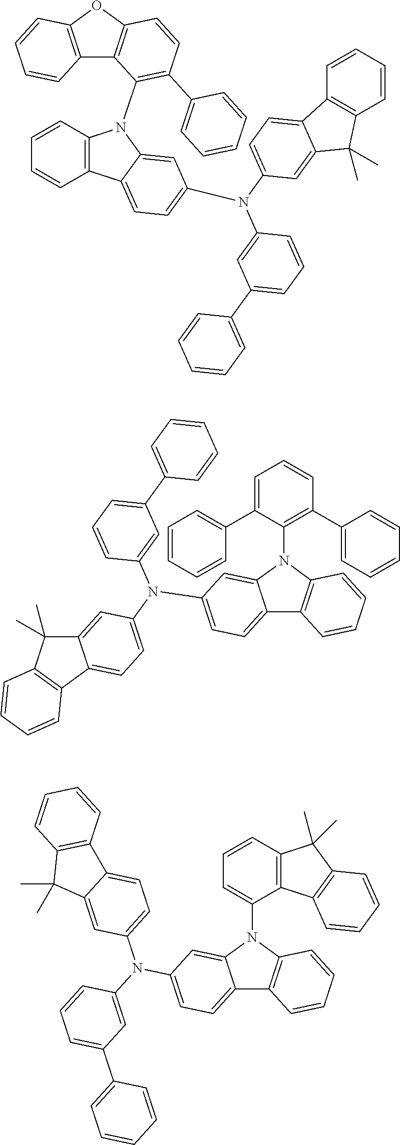 [SMILES: CC1(C)c2ccccc2-c2ccc(N(c3cccc(-c4ccccc4)c3)c3ccc4c5ccccc5n(-c5c(-c6ccccc6)ccc6oc7ccccc7c56)c4c3)cc21 ; CC1(C)c2ccccc2-c2ccc(N(c3cccc(-c4ccccc4)c3)c3ccc4c5ccccc5n(-c5c(-c6ccccc6)cccc5-c5ccccc5)c4c3)cc21 ; CC1(C)c2ccccc2-c2ccc(N(c3cccc(-c4ccccc4)c3)c3ccc4c5ccccc5n(-c5cccc6c5-c5ccccc5C6(C)C)c4c3)cc21]

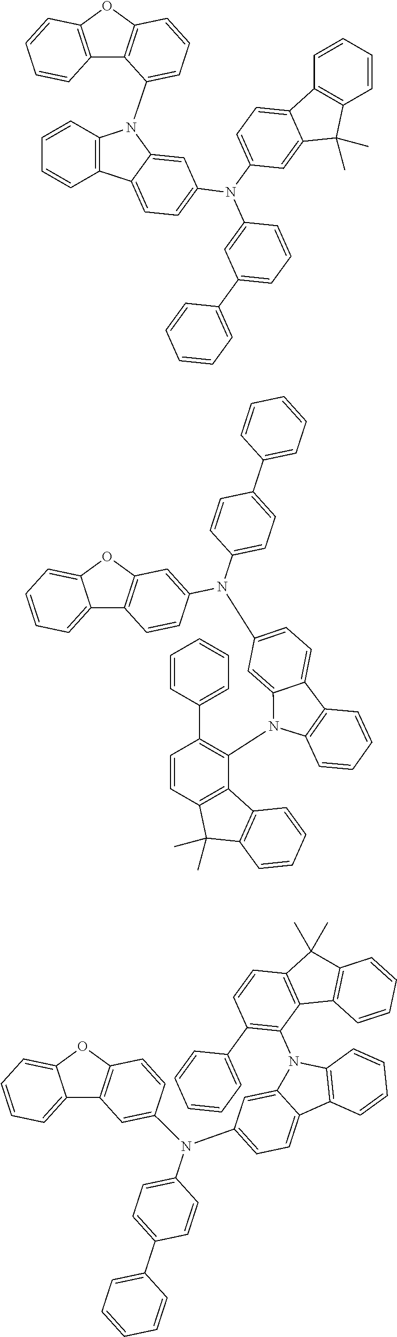 [SMILES: CC1(C)c2ccccc2-c2ccc(N(c3cccc(-c4ccccc4)c3)c3ccc4c5ccccc5n(-c5cccc6oc7ccccc7c56)c4c3)cc21 ; CC1(C)c2ccccc2-c2c1ccc(-c1ccccc1)c2-n1c2ccccc2c2ccc(N(c3ccc(-c4ccccc4)cc3)c3ccc4c(c3)oc3ccccc34)cc21 ; CC1(C)c2ccccc2-c2c1ccc(-c1ccccc1)c2-n1c2ccccc2c2ccc(N(c3ccc(-c4ccccc4)cc3)c3ccc4oc5ccccc5c4c3)cc21]

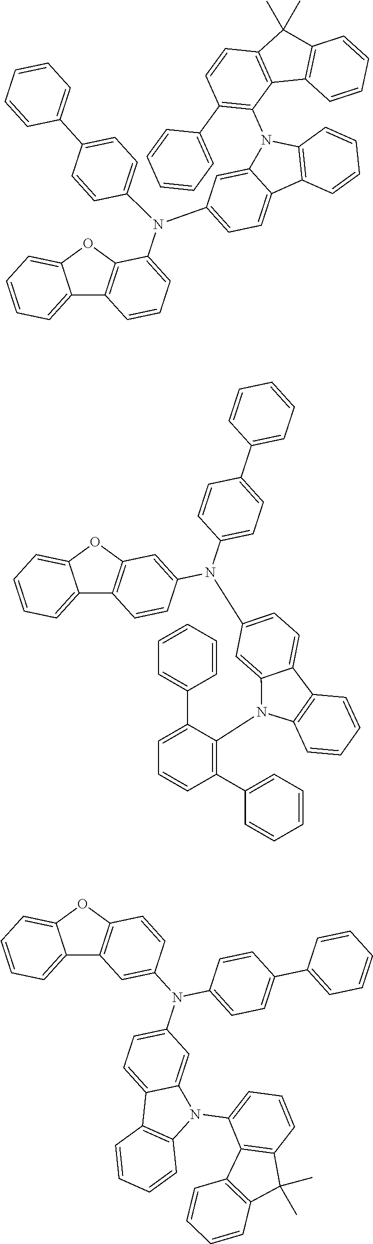 [SMILES: CC1(C)c2ccccc2-c2c1ccc(-c1ccccc1)c2-n1c2ccccc2c2ccc(N(c3ccc(-c4ccccc4)cc3)c3cccc4c3oc3ccccc34)cc21 ; c1ccc(-c2ccc(N(c3ccc4c(c3)oc3ccccc34)c3ccc4c5ccccc5n(-c5c(-c6ccccc6)cccc5-c5ccccc5)c4c3)cc2)cc1 ; CC1(C)c2ccccc2-c2c(-n3c4ccccc4c4ccc(N(c5ccc(-c6ccccc6)cc5)c5ccc6oc7ccccc7c6c5)cc43)cccc21]

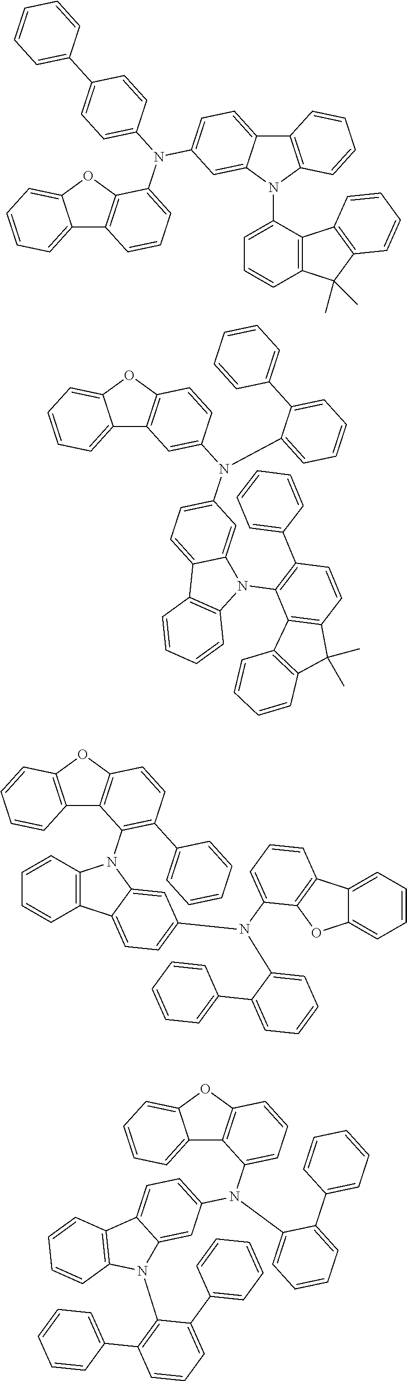

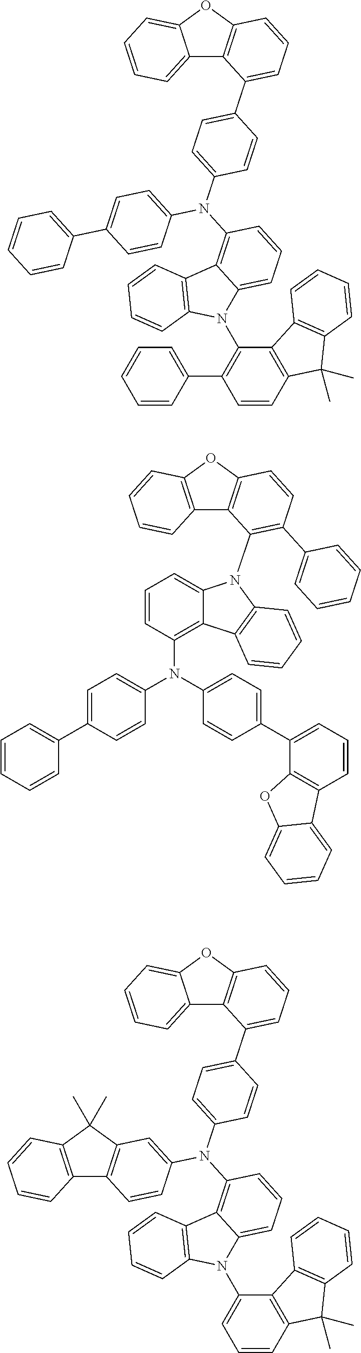 [SMILES: CC1(C)c2ccccc2-c2c1ccc(-c1ccccc1)c2-n1c2ccccc2c2c(N(c3ccc(-c4ccccc4)cc3)c3ccc(-c4cccc5oc6ccccc6c45)cc3)cccc21 ; c1ccc(-c2ccc(N(c3ccc(-c4cccc5c4oc4ccccc45)cc3)c3cccc4c3c3ccccc3n4-c3c(-c4ccccc4)ccc4oc5ccccc5c34)cc2)cc1 ; CC1(C)c2ccccc2-c2ccc(N(c3ccc(-c4cccc5oc6ccccc6c45)cc3)c3cccc4c3c3ccccc3n4-c3cccc4c3-c3ccccc3C4(C)C)cc21]

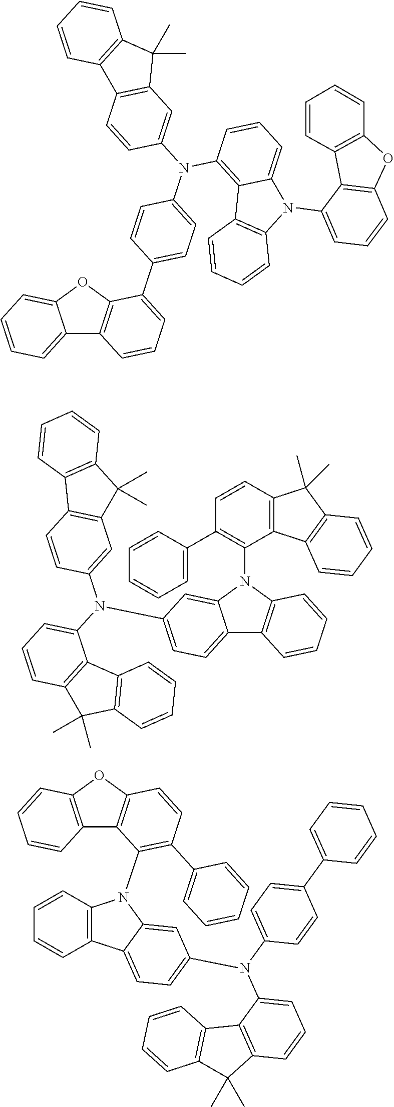

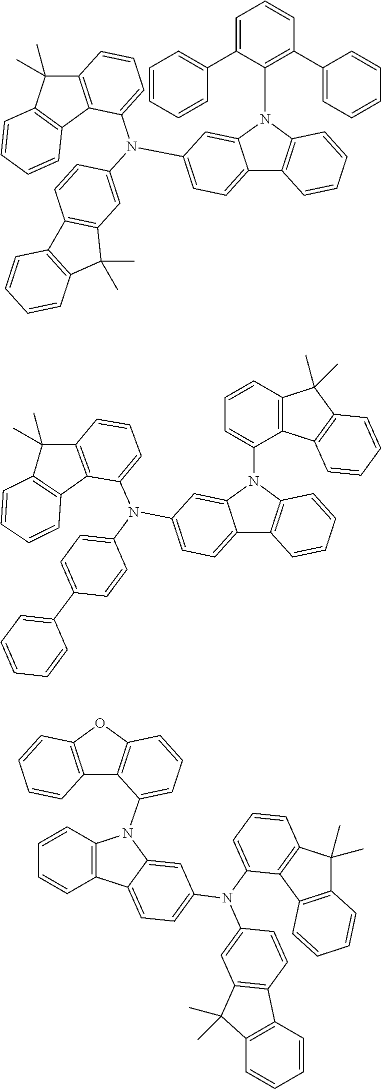 [SMILES: CC1(C)c2ccccc2-c2ccc(N(c3ccc4c5ccccc5n(-c5c(-c6ccccc6)cccc5-c5ccccc5)c4c3)c3cccc4c3-c3ccccc3C4(C)C)cc21 ; CC1(C)c2ccccc2-c2c(N(c3ccc(-c4ccccc4)cc3)c3ccc4c5ccccc5n(-c5cccc6c5-c5ccccc5C6(C)C)c4c3)cccc21 ; CC1(C)c2ccccc2-c2ccc(N(c3ccc4c5ccccc5n(-c5cccc6oc7ccccc7c56)c4c3)c3cccc4c3-c3ccccc3C4(C)C)cc21]

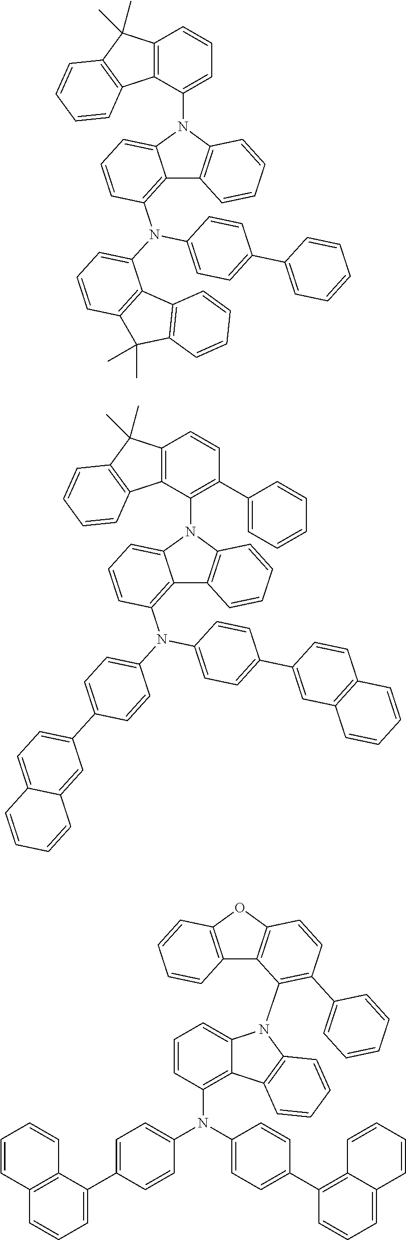

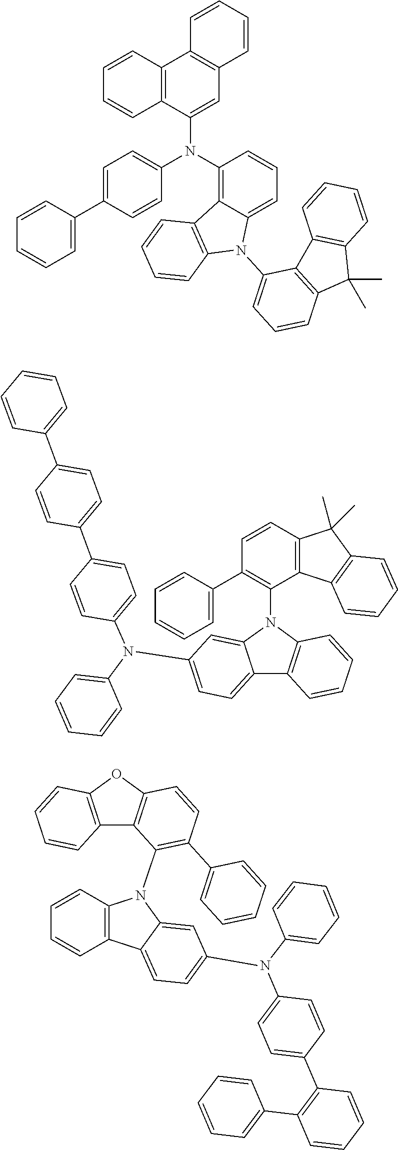

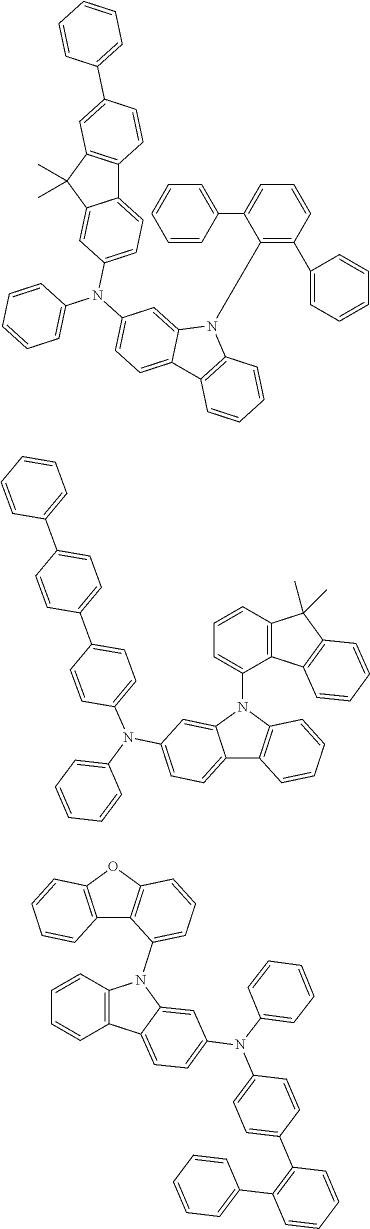

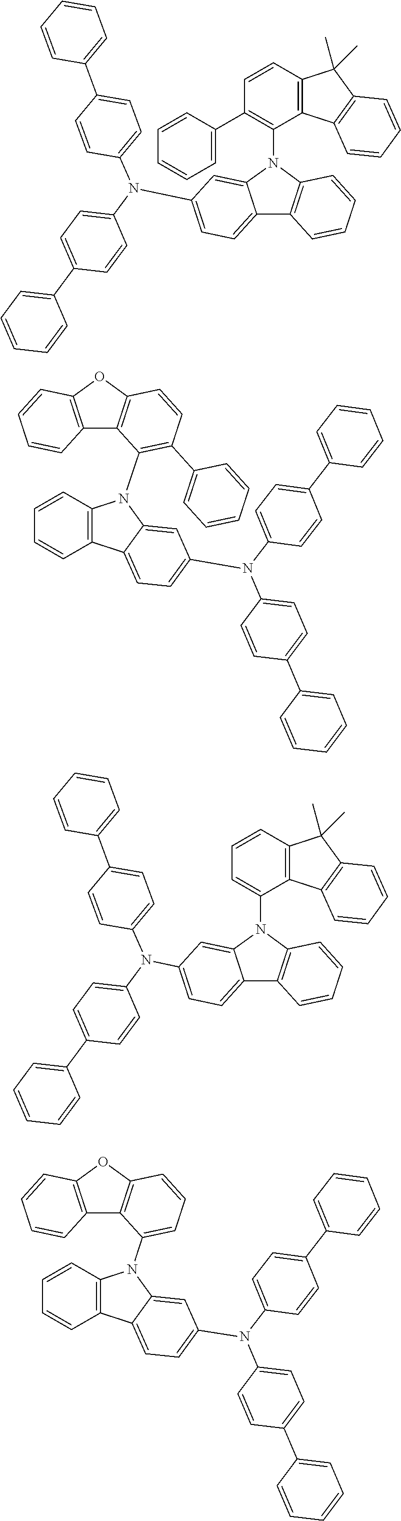

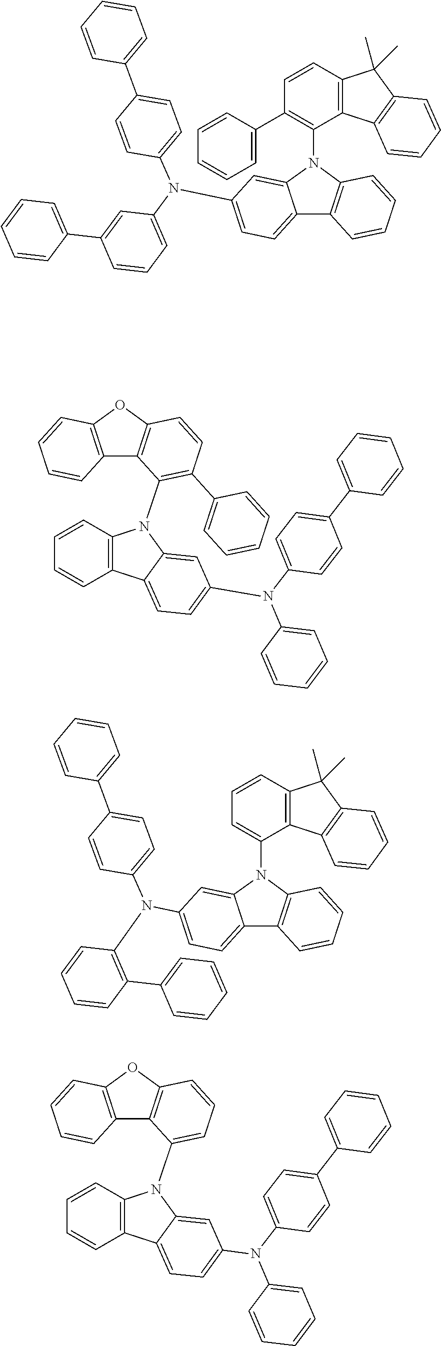 [SMILES: CC1(C)c2ccccc2-c2c1ccc(-c1ccccc1)c2-n1c2ccccc2c2ccc(N(c3ccc(-c4ccccc4)cc3)c3cccc(-c4ccccc4)c3)cc21 ; c1ccc(-c2ccc(N(c3ccccc3)c3ccc4c5ccccc5n(-c5c(-c6ccccc6)ccc6oc7ccccc7c56)c4c3)cc2)cc1 ; CC1(C)c2ccccc2-c2c(-n3c4ccccc4c4ccc(N(c5ccc(-c6ccccc6)cc5)c5ccccc5-c5ccccc5)cc43)cccc21 ; c1ccc(-c2ccc(N(c3ccccc3)c3ccc4c5ccccc5n(-c5cccc6oc7ccccc7c56)c4c3)cc2)cc1]

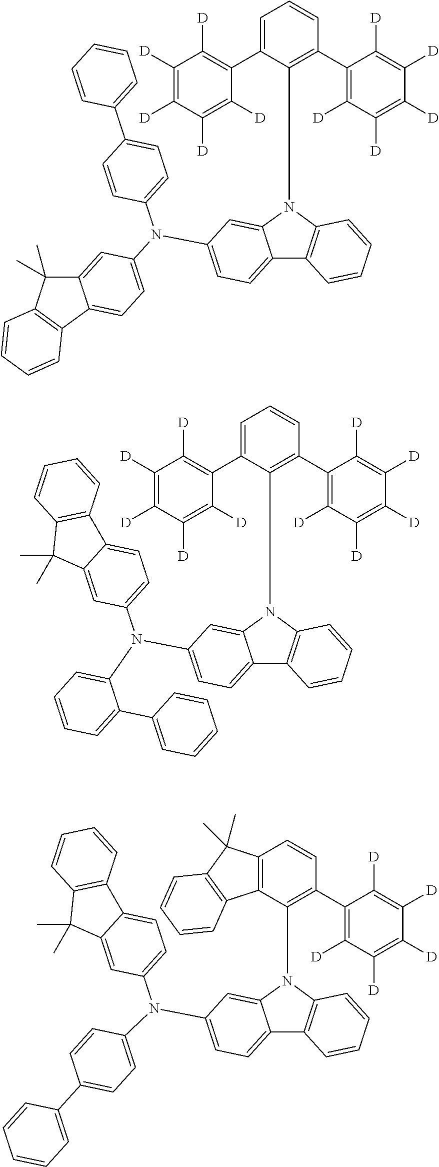 [SMILES: [2H]c1c([2H])c([2H])c(-c2cccc(-c3c([2H])c([2H])c([2H])c([2H])c3[2H])c2-n2c3ccccc3c3ccc(N(c4ccc(-c5ccccc5)cc4)c4ccc5c(c4)C(C)(C)c4ccccc4-5)cc32)c([2H])c1[2H] ; [2H]c1c([2H])c([2H])c(-c2cccc(-c3c([2H])c([2H])c([2H])c([2H])c3[2H])c2-n2c3ccccc3c3ccc(N(c4ccc5c(c4)C(C)(C)c4ccccc4-5)c4ccccc4-c4ccccc4)cc32)c([2H])c1[2H] ; [2H]c1c([2H])c([2H])c(-c2ccc3c(c2-n2c4ccccc4c4ccc(N(c5ccc(-c6ccccc6)cc5)c5ccc6c(c5)C(C)(C)c5ccccc5-6)cc42)-c2ccccc2C3(C)C)c([2H])c1[2H]]

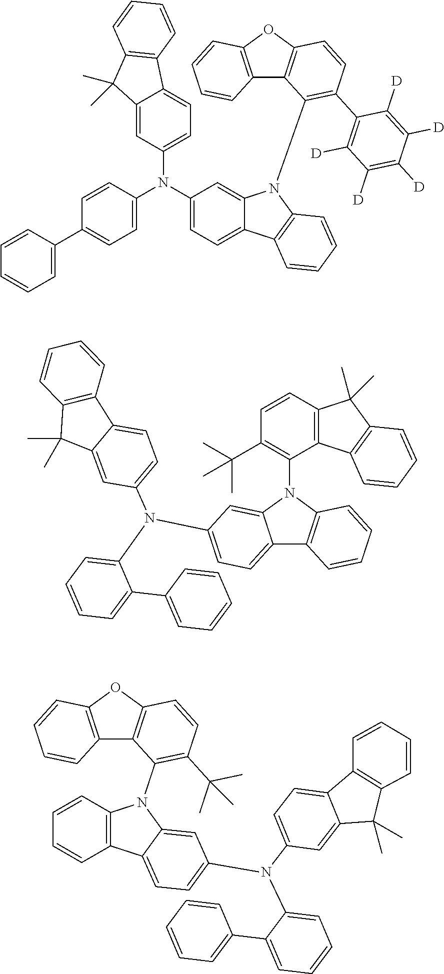 [SMILES: [2H]c1c([2H])c([2H])c(-c2ccc3oc4ccccc4c3c2-n2c3ccccc3c3ccc(N(c4ccc(-c5ccccc5)cc4)c4ccc5c(c4)C(C)(C)c4ccccc4-5)cc32)c([2H])c1[2H] ; CC(C)(C)c1ccc2c(c1-n1c3ccccc3c3ccc(N(c4ccc5c(c4)C(C)(C)c4ccccc4-5)c4ccccc4-c4ccccc4)cc31)-c1ccccc1C2(C)C ; CC(C)(C)c1ccc2oc3ccccc3c2c1-n1c2ccccc2c2ccc(N(c3ccc4c(c3)C(C)(C)c3ccccc3-4)c3ccccc3-c3ccccc3)cc21]

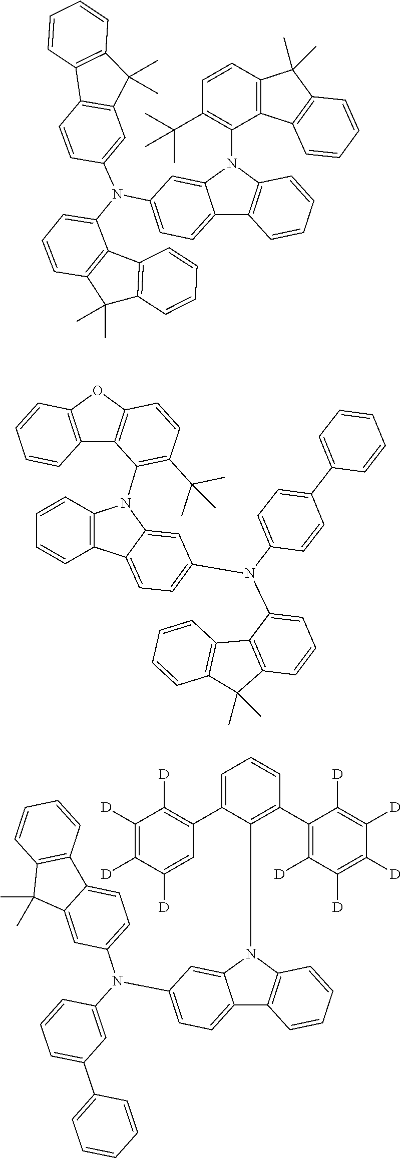

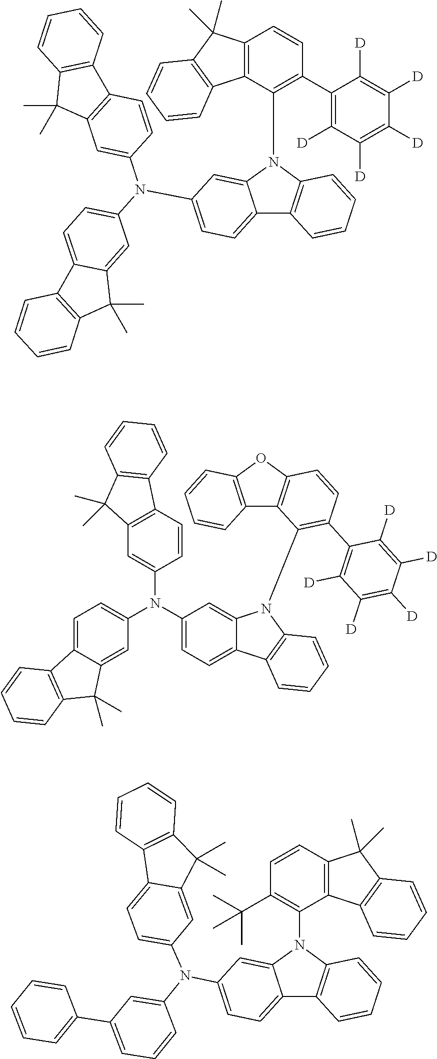 [SMILES: [2H]c1c([2H])c([2H])c(-c2ccc3c(c2-n2c4ccccc4c4ccc(N(c5ccc6c(c5)C(C)(C)c5ccccc5-6)c5ccc6c(c5)C(C)(C)c5ccccc5-6)cc42)-c2ccccc2C3(C)C)c([2H])c1[2H] ; [2H]c1c([2H])c([2H])c(-c2ccc3oc4ccccc4c3c2-n2c3ccccc3c3ccc(N(c4ccc5c(c4)C(C)(C)c4ccccc4-5)c4ccc5c(c4)C(C)(C)c4ccccc4-5)cc32)c([2H])c1[2H] ; CC(C)(C)c1ccc2c(c1-n1c3ccccc3c3ccc(N(c4cccc(-c5ccccc5)c4)c4ccc5c(c4)C(C)(C)c4ccccc4-5)cc31)-c1ccccc1C2(C)C]

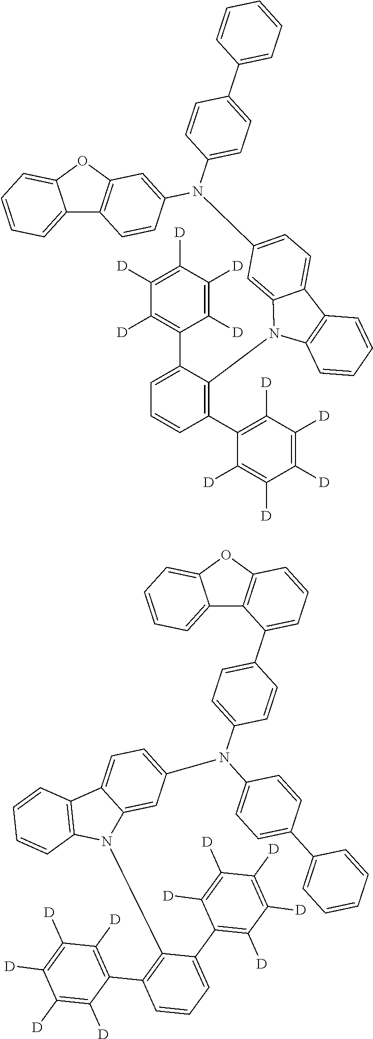 [SMILES: [2H]c1c([2H])c([2H])c(-c2cccc(-c3c([2H])c([2H])c([2H])c([2H])c3[2H])c2-n2c3ccccc3c3ccc(N(c4ccc(-c5ccccc5)cc4)c4ccc5c(c4)oc4ccccc45)cc32)c([2H])c1[2H] ; [2H]c1c([2H])c([2H])c(-c2cccc(-c3c([2H])c([2H])c([2H])c([2H])c3[2H])c2-n2c3ccccc3c3ccc(N(c4ccc(-c5ccccc5)cc4)c4ccc(-c5cccc6oc7ccccc7c56)cc4)cc32)c([2H])c1[2H]]

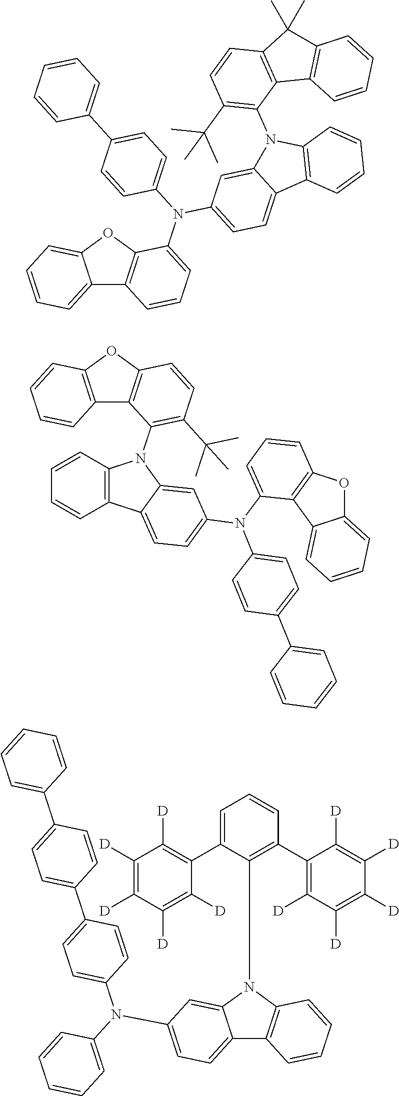 [SMILES: CC(C)(C)c1ccc2c(c1-n1c3ccccc3c3ccc(N(c4ccc(-c5ccccc5)cc4)c4cccc5c4oc4ccccc45)cc31)-c1ccccc1C2(C)C ; CC(C)(C)c1ccc2oc3ccccc3c2c1-n1c2ccccc2c2ccc(N(c3ccc(-c4ccccc4)cc3)c3cccc4oc5ccccc5c34)cc21 ; [2H]c1c([2H])c([2H])c(-c2cccc(-c3c([2H])c([2H])c([2H])c([2H])c3[2H])c2-n2c3ccccc3c3ccc(N(c4ccccc4)c4ccc(-c5ccc(-c6ccccc6)cc5)cc4)cc32)c([2H])c1[2H]]

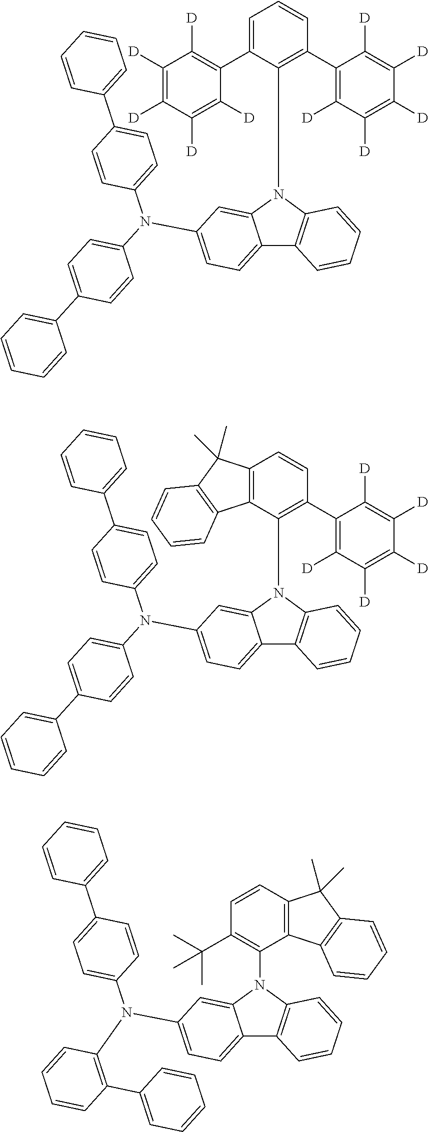 [SMILES: [2H]c1c([2H])c([2H])c(-c2cccc(-c3c([2H])c([2H])c([2H])c([2H])c3[2H])c2-n2c3ccccc3c3ccc(N(c4ccc(-c5ccccc5)cc4)c4ccc(-c5ccccc5)cc4)cc32)c([2H])c1[2H] ; [2H]c1c([2H])c([2H])c(-c2ccc3c(c2-n2c4ccccc4c4ccc(N(c5ccc(-c6ccccc6)cc5)c5ccc(-c6ccccc6)cc5)cc42)-c2ccccc2C3(C)C)c([2H])c1[2H] ; CC(C)(C)c1ccc2c(c1-n1c3ccccc3c3ccc(N(c4ccc(-c5ccccc5)cc4)c4ccccc4-c4ccccc4)cc31)-c1ccccc1C2(C)C]

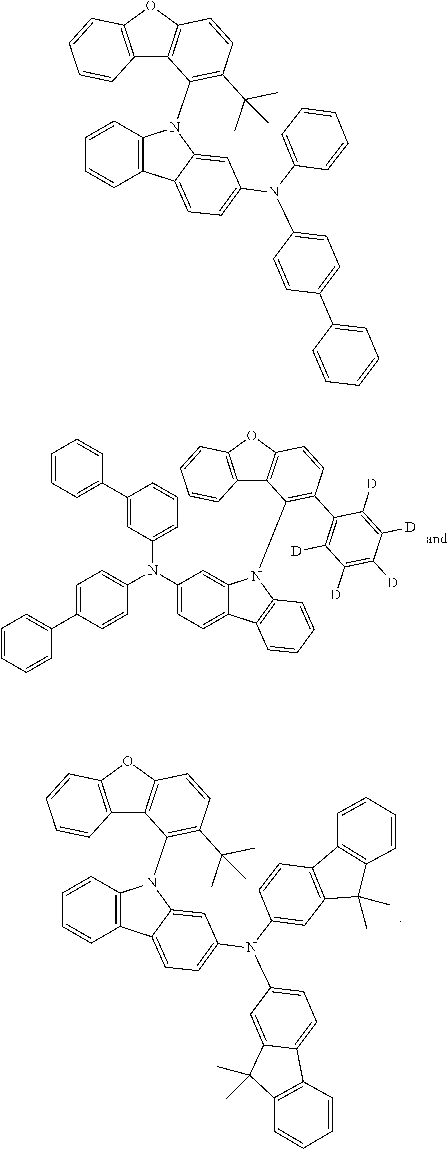 [SMILES: CC(C)(C)c1ccc2oc3ccccc3c2c1-n1c2ccccc2c2ccc(N(c3ccccc3)c3ccc(-c4ccccc4)cc3)cc21 ; [2H]c1c([2H])c([2H])c(-c2ccc3oc4ccccc4c3c2-n2c3ccccc3c3ccc(N(c4ccc(-c5ccccc5)cc4)c4cccc(-c5ccccc5)c4)cc32)c([2H])c1[2H] ; CC(C)(C)c1ccc2oc3ccccc3c2c1-n1c2ccccc2c2ccc(N(c3ccc4c(c3)C(C)(C)c3ccccc3-4)c3ccc4c(c3)C(C)(C)c3ccccc3-4)cc21]

In a second aspect, the present invention provides an application of the above-mentioned compound containing the carbazole derivative in the preparation of an organic electroluminescent device.

In a third aspect, the present invention provides an organic electroluminescent device, wherein the organic electroluminescent device comprises one or more compounds containing carbazole derivatives as provided in the first aspect of the present invention.

In conjunction with the third aspect, the organic electroluminescent device comprises an anode, a hole transport layer, a luminescent auxiliary layer, a luminescent layer, an electron transport layer, an electron injection layer, and a cathode, which are sequentially arranged on the substrate, wherein the hole transport layer comprises one or more compounds containing carbazole derivatives as provided in the first aspect of the present invention.

### Beneficial Effects of the Invention

The compound containing the carbazole derivative as provided by the present invention has a fluorene structure, which is beneficial to delocalization of electrons in the molecule, can effectively reduce the hole transport barrier potential and increase the hole transport rate. In the compound containing the carbazole derivative as provided by the present invention, by introducing phenyl, which has a relatively strong electron-donating ability, in the ortho positions of the phenyl group connected to carbazole, the HOMO energy level value of the compound can be improved, the steric hindrance of the molecule can be significantly increased, the molecular rotation during heating is reduced, and the glass transition temperature of the molecule is increased. The compound containing the carbazole derivative as provided by the present invention can be formed with a crisscross spatial structure by introducing a fluorene structure in the ortho position of the phenyl connected to carbazole, which is beneficial to the stacking between molecules and facilitates the transport of holes between molecules. In the compound containing the carbazole derivative as provided by the present invention, the diarylamine is directly connected to the benzene rings of the carbazole, whereby the conjugation of two benzene rings in the carbazole is better, which can improve the hole transport rate, and the carbazole has a relatively strong electron-donating ability, which can significantly improve the HOMO energy level value of the compound. Therefore, the compound containing the carbazole derivative as provided by the present invention has a relatively high hole transport rate, a relatively high HOMO energy level value and a relatively high glass transition temperature. Where the compound containing the carbazole derivative as provided by the present invention is applied to the hole transport layer of an organic electroluminescent device, the hole transport layer has a relatively high HOMO energy level value, so that the interface barrier potential between the ITO film and the hole transport layer can be reduced, and the hole injection barrier potential can be reduced, thereby effectively reducing the driving voltage of the organic electroluminescent device. The hole transport layer has a relatively high hole transport rate, which can effectively improve the hole injection and migration efficiency in the organic electroluminescent device and ensure the maximum recombination between carriers in the luminescent layer, thus ensuring that the organic electroluminescent device attains the excellent effects of a high luminous efficiency and a low starting voltage. The hole transport layer has a relatively high glass transition temperature, a relatively good thermal stability and less possibility of crystallization or agglomeration, thereby enabling prolonged lifetime of the organic electroluminescent device and overcoming the defects in the prior art. It is thus indicated that the compound containing the carbazole derivative as provided by the present invention is a hole transport layer material with a good performance, which can meet the performance requirements of organic electroluminescent devices and has practical value.

### BRIEF DESCRIPTION OF THE DRAWINGS

The specific embodiments of the present invention will be described in further detail with reference to the accompanying drawings.

FIG. 1 is a schematic structural diagram of an organic electroluminescent device comprising the polycyclic compound of the present invention.

In the Brief Description of the Drawings: 1—substrate, 2—anode, 3—hole transport layer, 4—luminescent auxiliary layer, 5—luminescent layer, 6—electron transport layer, 7—electron injection layer, and 8—cathode.

### DETAILED DESCRIPTION OF EMBODIMENTS

In order to explain the present invention more clearly, the present invention will be further explained below in conjunction with preferred examples and the accompanying drawings. In the drawings, like parts are denoted by like reference signs. A person skilled in the art should understand that the following detailed description is illustrative rather than restrictive and should not limit the scope of protection of the present invention. The examples and comparative examples in the present description are provided to explain the present description more completely to those skilled in the art. The examples and comparative examples according to the present description can be transformed into various forms, and the scope of protection of the present invention should not be limited to the examples and comparative examples detailed below.

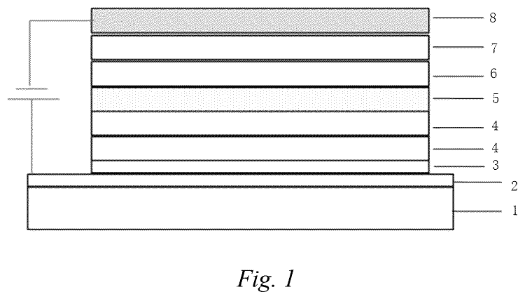

The compound of the present invention is suitable for light-emitting elements, display panels, and electronic devices, especially for organic electroluminescent devices. The electronic device of the present invention is a device that comprises a layer of at least one organic compound, and the device may also comprise an inorganic material or a layer formed entirely of an inorganic material. The electronic device is preferably an organic electroluminescent device (OLED), an organic integrated circuit (O-IC), an organic field-effect transistor (O-FET), an organic thin film transistor (O-TFT), an organic light-emitting transistor (O-LET), an organic solar cell (O-SC), an organic dye-sensitized solar cell (O-DSSC), an organic optical detector, an organic photosensor, an organic field-quenching device (O-FQD), a luminescent electrochemical cell (LEC), an organic laser diode (O-laser), and an organic plasma emitting device. The electronic device is preferably an organic electroluminescent device (OLED). The schematic structural diagram of an exemplary organic electroluminescent device is as shown in FIG. 1.

The polycyclic aromatic amine compound as provided by the present invention is prepared by means of Buchwald-Hartwig coupling reaction, Suzuki coupling reaction, or Heck coupling reaction as representative reactions.

In order to understand the content of the present invention more clearly, the compound, the preparation method for the compound, and the luminescent characteristics of the device comprising the compound will be explained in detail in conjunction with examples. Various chemical reactions can be applied to the synthesis method for a compound according to one embodiment of the present invention. However, it should be noted that the synthesis method for the compound according to one embodiment of the present invention is not limited to the synthesis method described below. Unless otherwise specified, the subsequent synthesis is carried out in an anhydrous solvent in a protective gas atmosphere. Solvents and reagents can be purchased from conventional reagent suppliers.

### INTERMEDIATE SYNTHESIS EXAMPLES

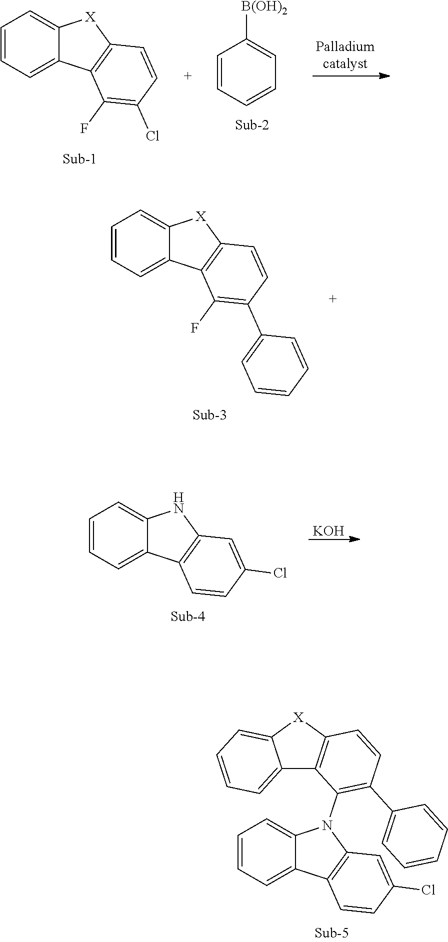

Sub-1 and Sub-2 (the molar ratio of Sub-1 to Sub-2 was 1:1) were added to a mixed solvent of toluene and water (the volume ratio of toluene to water was 5:1), 0.1 equivalent of [1,1′-bis(diphenylphosphino)ferrocene]palladium dichloride and 2 equivalents of potassium carbonate were added, the mixture was stirred for 8 hours under reflux under nitrogen protection, and the mixture resulting from the reaction was purified by column chromatography to obtain Sub-3.

Sub-3 and Sub-4 (the molar ratio of Sub-3 to Sub-4 was 1:1) were added to dimethyl sulfoxide, 1.5 equivalents of potassium hydroxide was added, the mixture was stirred for 24 hours under reflux at 160° C., and the mixture resulting from the reaction was purified by column chromatography to obtain Sub-5.

Other intermediates B1 to B10 could be prepared by a method similar to the preparation of Sub-5.

### COMPOUND SYNTHESIS EXAMPLES

### Synthesis Example 1

This example provided Compound C1 containing a carbazole derivative, and the synthesis route of the compound was as follows.

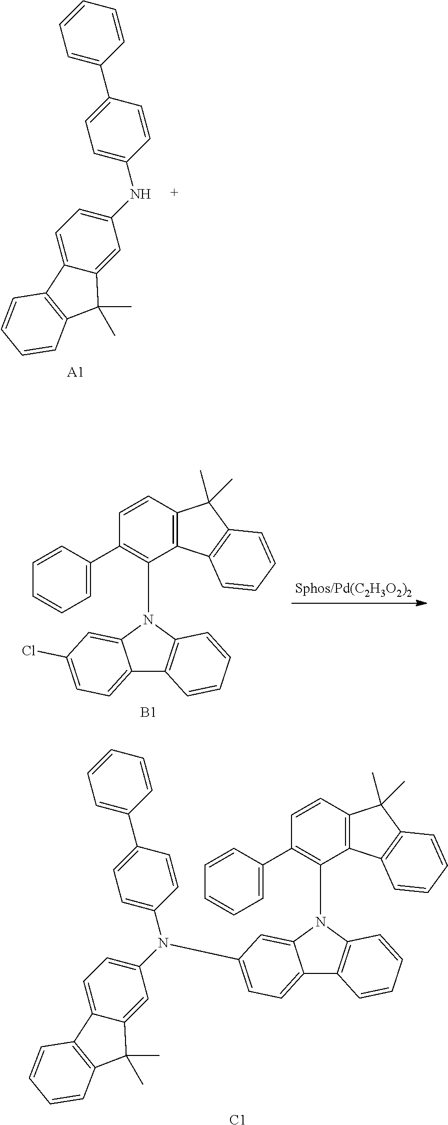

A1 (2.6 g, 10 mmol), B1 (4.70 g, 10 mmol), and sodium tert-butoxide (1.06 g, 11 mmol) were added to toluene (50 mL), and palladium acetate (23 mg, 0.1 mmol) and Sphos (82 mg, 0.2 mmol) were then introduced under nitrogen protection. Subsequently, the reaction system was heated to 110° C., refluxed and maintained for 8 hours. After cooling to room temperature, the reaction system was quenched with water and separated. The organic phase was dried with anhydrous sodium sulfate and subjected to rotary evaporation to remove the solvent to obtain a crude product, and the crude product was separated and purified by column chromatography (the mobile phase for the column chromatography was petroleum ether:ethyl acetate=15:1). Finally, the product C1 was obtained: 4.36 g (yield: 62%), MS (m/z) (M+): 795.0.

### Synthesis Example 2

This example provided Compound C2 containing a carbazole derivative, and the synthesis route of the compound was as follows.

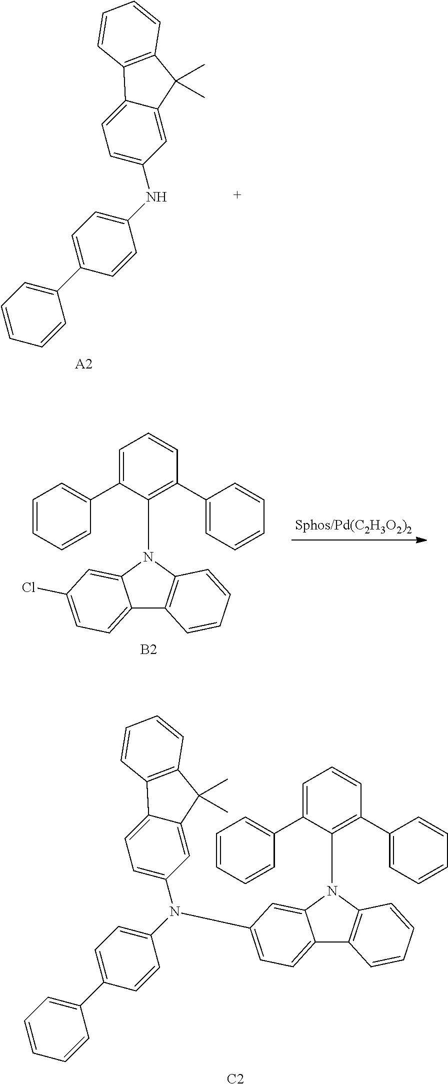

The same method as in Synthesis Example 1 was used, except that A1 and B1 were replaced with A2 (3.62 g, 10 mmol) and B2 (4.30 g, 10 mmol) to finally obtain the product C2: 4.72 g (yield: 67%), MS (m/z) (M+): 704.

### Synthesis Example 3

This example provided Compound C3 containing a carbazole derivative, and the synthesis route of the compound was as follows.

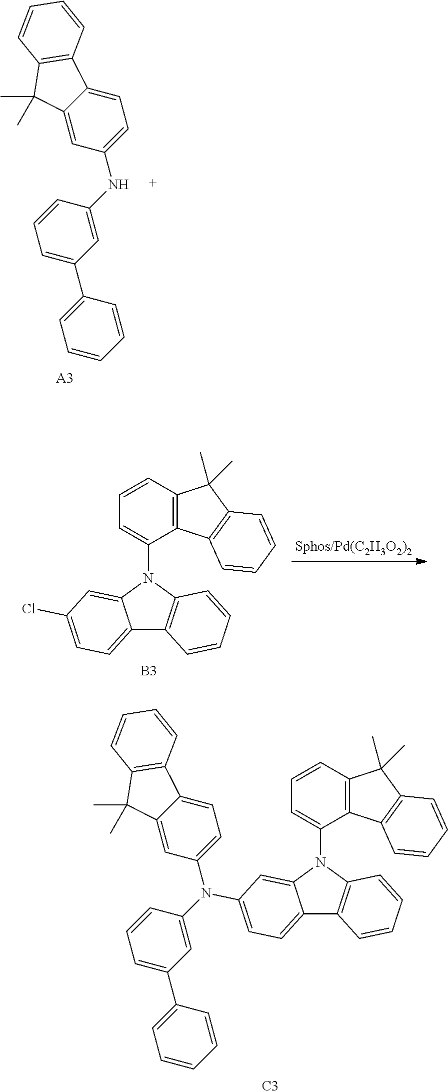

The same method as in Synthesis Example 1 was used, except that A1 and B1 were replaced with A3 (3.62 g, 10 mmol) and B3 (3.94 g, 10 mmol) to finally obtain the product C3: 3.99 g (yield: 55.6%), MS (m/z) (M+): 718.9.

### Synthesis Example 4

This example provided Compound C4 containing a carbazole derivative, and the synthesis route of the compound was as follows.

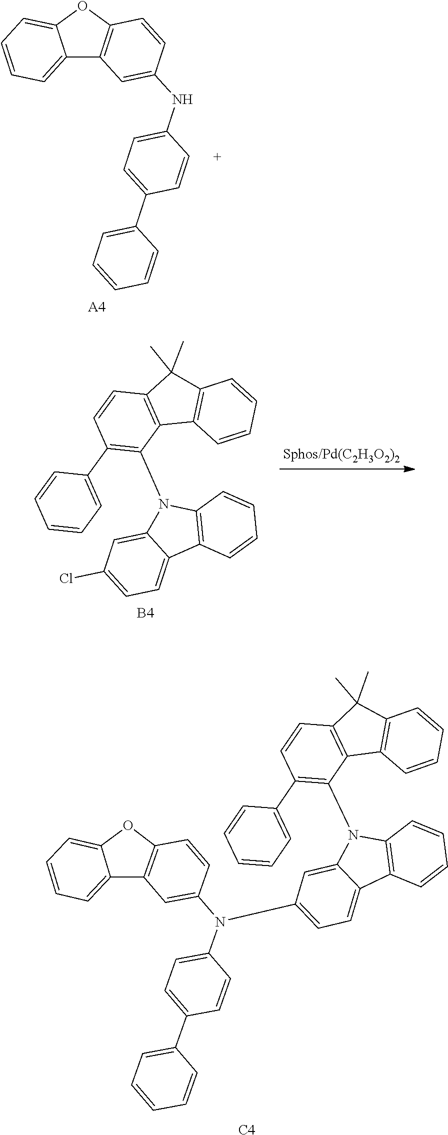

The same method as in Synthesis Example 1 was used, except that A1 and B1 were replaced with A4 (3.35 g, 10 mmol) and B4 (4.70 g, 10 mmol) to finally obtain the product C4: 4.63 g (yield: 60.2%), MS (m/z) (M+): 769.0.

### Synthesis Example 5

This example provided Compound C5 containing a carbazole derivative, and the synthesis route of the compound was as follows.

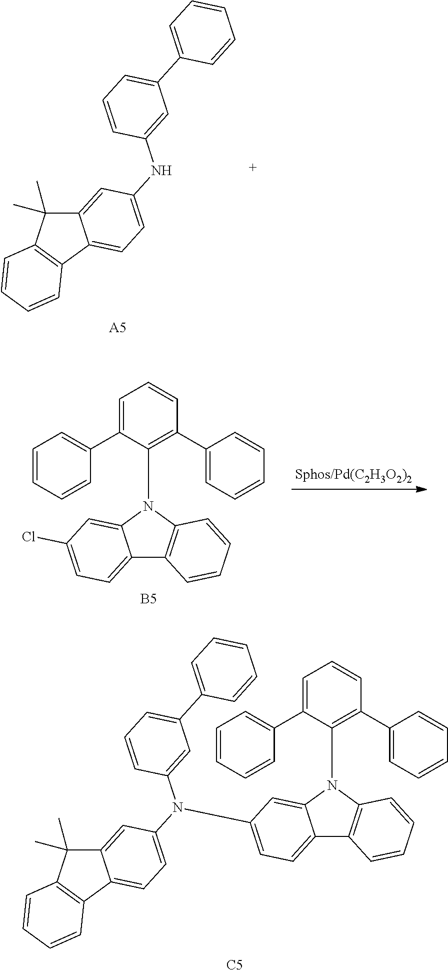

The same method as in Synthesis Example 1 was used, except that A1 and B1 were replaced with A5 (3.62 g, 10 mmol) and B5 (4.30 g, 10 mmol) to finally obtain the product C5: 5.27 g (yield: 69.8%), MS (m/z) (M+): 755.0.

### Synthesis Example 6

This example provided Compound C6 containing a carbazole derivative, and the synthesis route of the compound was as follows.

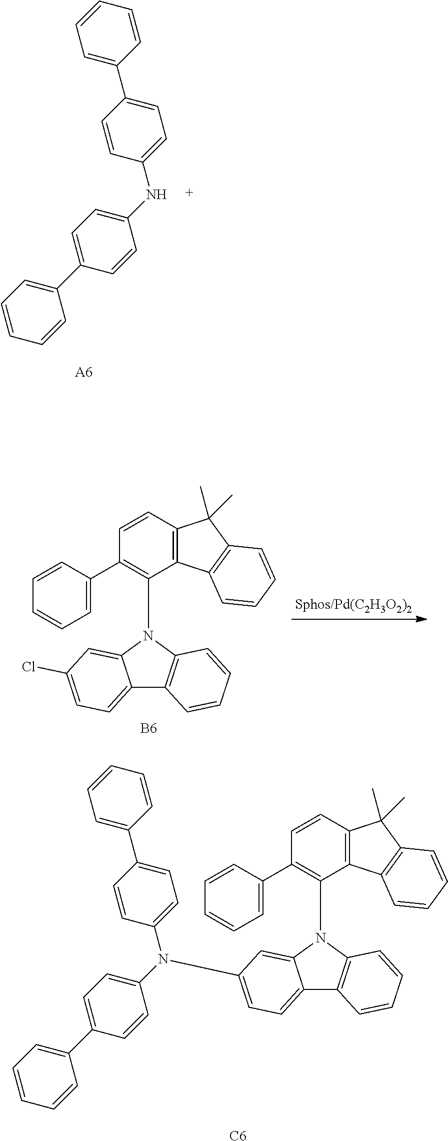

The same method as in Synthesis Example 1 was used, except that A1 and B1 were replaced with A6 (3.21 g, 10 mmol) and B6 (4.70 g, 10 mmol) to finally obtain the product C6: 5.15 g (yield: 68.2%), MS (m/z) (M+): 755.0.

### Synthesis Example 7

This example provided Compound C7 containing a carbazole derivative, and the synthesis route of the compound was as follows.

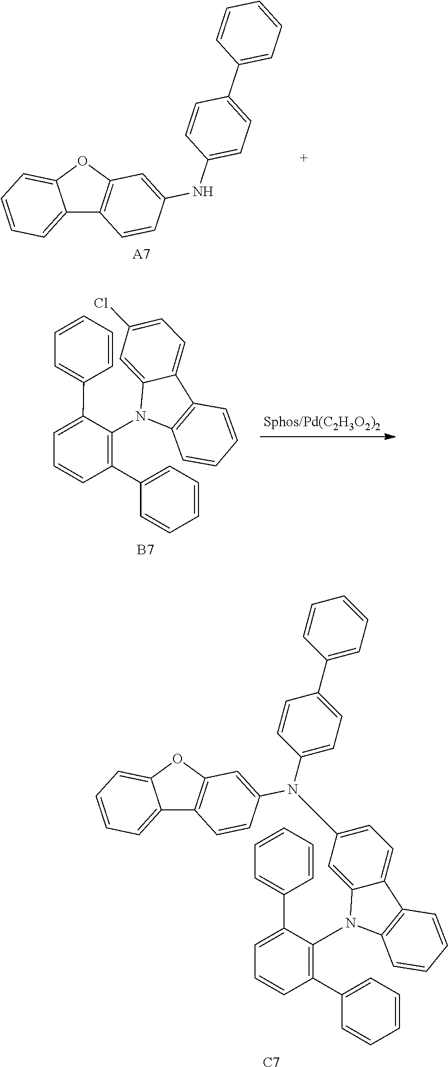

The same method as in Synthesis Example 1 was used, except that A1 and B1 were replaced with A7 (3.55 g, 10 mmol) and B7 (4.3 g, 10 mmol) to finally obtain the product C7: 3.67 g (yield: 50.3%), MS (m/z) (M+): 728.9.

### Synthesis Example 8

This example provided Compound C8 containing a carbazole derivative, and the synthesis route of the compound was as follows.

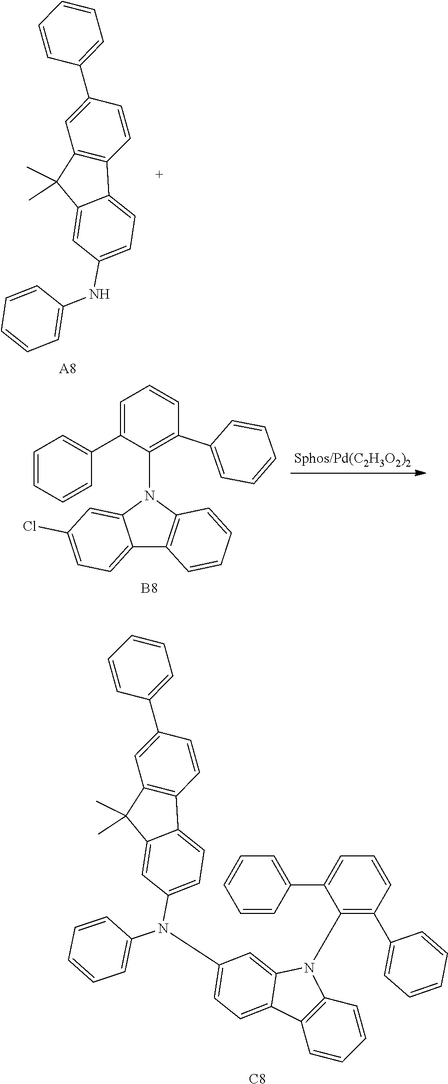

The same method as in Synthesis Example 1 was used, except that A1 and B1 were replaced with A8 (3.61 g, 10 mmol) and B8 (4.29 g, 10 mmol) to finally obtain the product C8: 5.47 g (yield: 70.6%), MS (m/z) (M+): 775.0.

### Synthesis Example 9

This example provided Compound C9 containing a carbazole derivative, and the synthesis route of the compound was as follows.

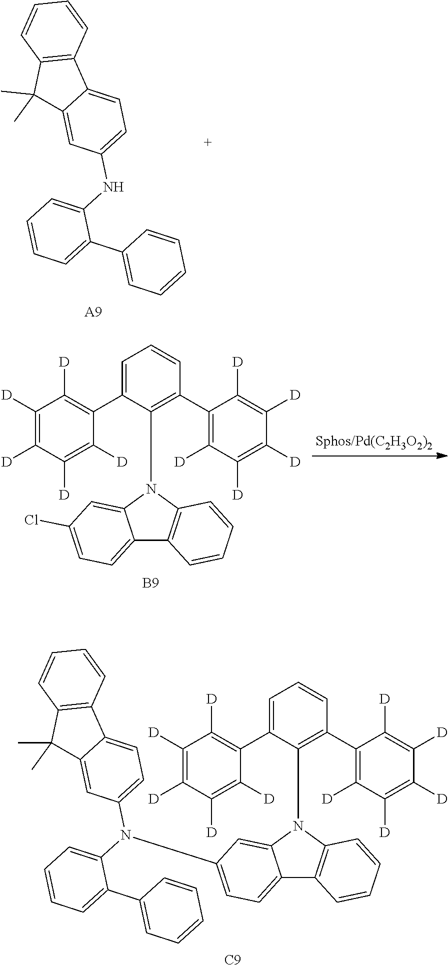

The same method as in Synthesis Example 1 was used, except that A1 and B1 were replaced with A9 (3.62 g, 10 mmol) and B9 (4.4 g, 10 mmol) to finally obtain the product C9: 5.03 g (yield: 65.7%), MS (m/z) (M+): 765.0.

### Synthesis Example 10

This example provided Compound C10 containing a carbazole derivative, and the synthesis route of the compound was as follows.

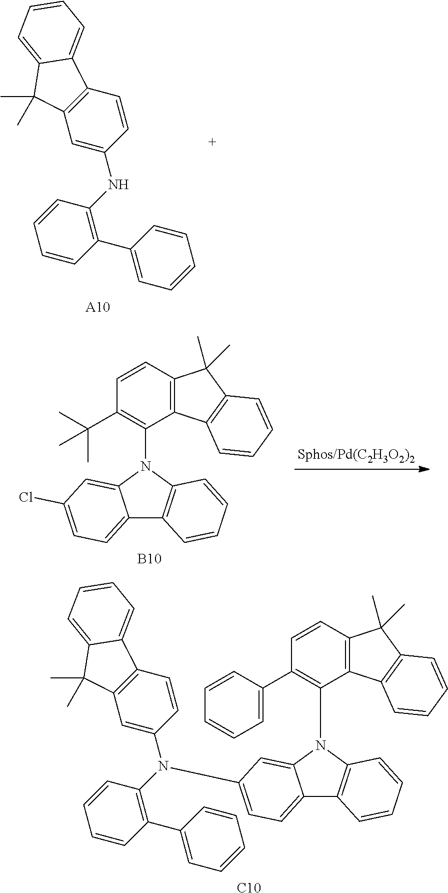

The same method as in Synthesis Example 1 was used, except that A1 and B1 were replaced with A10 (3.62 g, 10 mmol) and B10 (4.5 g, 10 mmol) to finally obtain the product C10: 4.26 g (yield: 55.0%), MS (m/z) (M+): 775.1.

### Comparative Examples 1-5

This comparative example provided Compounds D1, D2, D3, D4 and D5 which had been experimented in the research process, and the specific structural formulas thereof were:

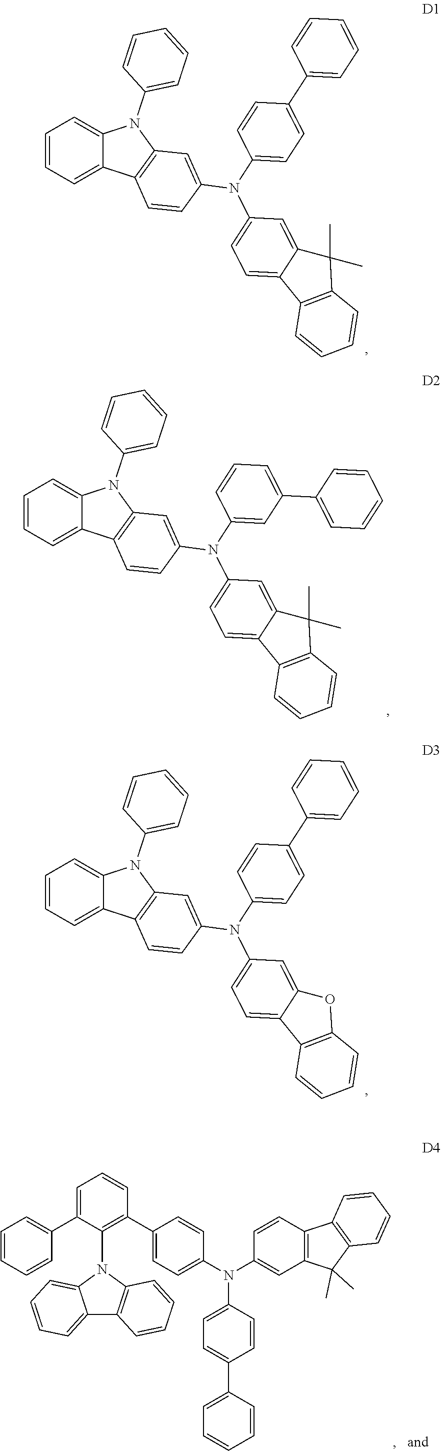

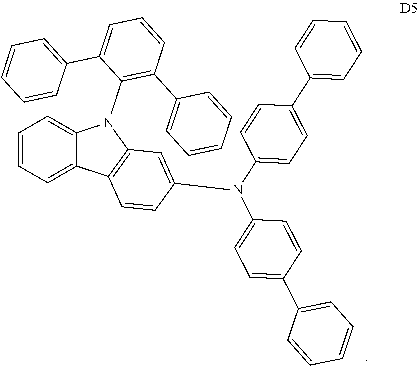 [SMILES: c1ccc(-c2ccc(N(c3ccc(-c4ccccc4)cc3)c3ccc4c5ccccc5n(-c5c(-c6ccccc6)cccc5-c5ccccc5)c4c3)cc2)cc1]

### Evaluation of Performance of Compounds

### Effect Example 1

In order to explain that the compound containing the carbazole derivative as provided by the present invention had relatively good properties, the compounds C1 to C10 provided by Compound Examples 1 to 10 of the present invention and the compounds D1 to D5 provided by Comparative Examples 1 to 5 were subjected to an HOD test, HOMO energy level calculation, and a thermal stability test, respectively.

### (1) HOD Test:

### Preparation Method for Sample to be Tested:

Firstly, on an ITO layer (anode) formed on a substrate, Compound C1 provided by Compound Example 1 and F4TCNQ (the mass ratio of Compound C1 to F4TCNQ was 97:3) were deposited in vacuo with a thickness of 10 nm to form a hole injection layer; secondly, on the above hole injection layer, Compound C1 was deposited in vacuo with a thickness of 120 nm to form a hole transport layer; and finally, aluminum (A1) was deposited with a thickness of 150 nm to form a cathode, thereby preparing a sample to be tested.

The preparation method for the remaining samples to be tested were as follows. Compound C1 in the above preparation method was replaced by Compounds C2 to C10 and Comparative Compounds D1 to D5, respectively, and the other preparation steps were the same.

### Specific HOD Test Method:

A voltage was applied to the samples to be tested separately. When the current passing through the sample to be tested was 10000 mA/cm², the applied voltage was determined. The HOD test could measure the speed of hole transport from the positive electrode to the luminescent layer. The higher the voltage, the slower the hole transport rate of the compound in the sample to be tested; conversely, the lower the voltage, the faster the hole transport rate of the compound in the sample to be tested.

### (2) HOMO Energy Level:

The compound to be tested and NPB were prepared into a thin film, the ionization potential value thereof was measured by means of a photoelectron spectrophotometer in the atmosphere, and the value thereof was further converted to obtain an HOMO energy level value.

### (3) Thermal Stability Test:

NETZSCH 3500 differential scanning calorimeter was used, wherein the nitrogen flow rate was 30.0 ml/min, the test temperature was 30° C. to 250° C., and the heating rate was 20° C./min. According to the test curve, the glass transition temperature (Tg) of the material was calculated. The glass transition temperature (Tg) of the material was associated with the film-forming property and thermal stability of the material. The higher the glass transition temperature of the material, the better the thermal stability of the material. The glass transition temperature (Tg) of the material was associated with the film-forming property and thermal stability of the material. The higher the glass transition temperature of the material, the better the thermal stability of the material, and the material was less prone to crystallization or agglomeration.

The above specific test and calculation results were as shown in Table 1:

TABLE 1
Performance test results of compounds

| | | | | Glass |
|---|---|---|---|---|
| | | | | transition |
| | | Voltage | HOMO | temperature |
| No. | Structure of compound | (V) | (eV) | (° C.) |
| C1 | 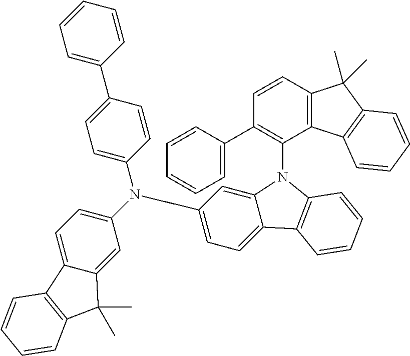 [SMILES: CC1(C)c2ccccc2-c2ccc(N(c3ccc(-c4ccccc4)cc3)c3ccc4c5ccccc5n(-c5c(-c6ccccc6)ccc6c5-c5ccccc5C6(C)C)c4c3)cc21] | 2.17 | −5.02 | 135 |
| | C1 | | | |
| C2 | 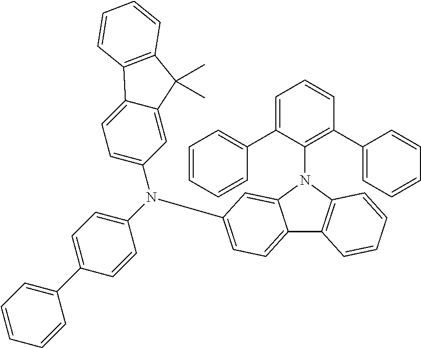 [SMILES: CC1(C)c2ccccc2-c2ccc(N(c3ccc(-c4ccccc4)cc3)c3ccc4c5ccccc5n(-c5c(-c6ccccc6)cccc5-c5ccccc5)c4c3)cc21] | 2.13 | −5.05 | 126 |
| | C2 | | | |
| C3 | 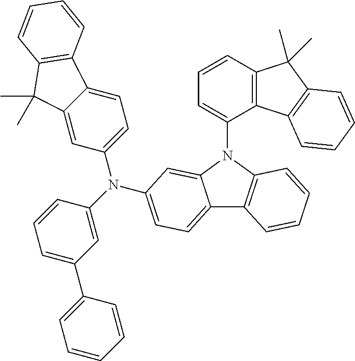 [SMILES: CC1(C)c2ccccc2-c2ccc(N(c3cccc(-c4ccccc4)c3)c3ccc4c5ccccc5n(-c5cccc6c5-c5ccccc5C6(C)C)c4c3)cc21] | 2.78 | −5.12 | 129 |
| | C3 | | | |
| C4 | 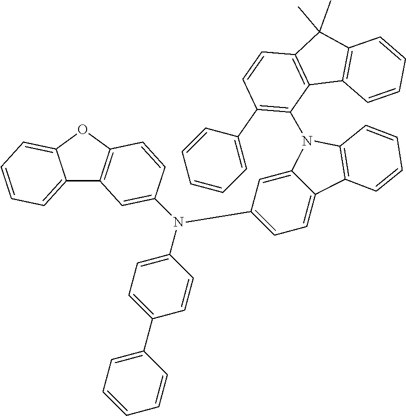 [SMILES: CC1(C)c2ccccc2-c2c1ccc(-c1ccccc1)c2-n1c2ccccc2c2ccc(N(c3ccc(-c4ccccc4)cc3)c3ccc4oc5ccccc5c4c3)cc21] | 2.56 | −4.93 | 128 |
| | C4 | | | |
| C5 | 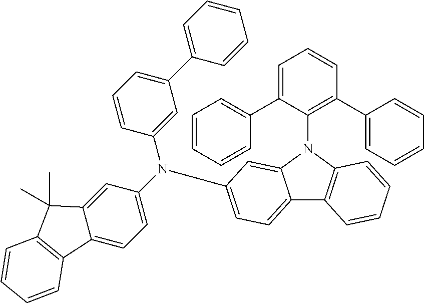 [SMILES: CC1(C)c2ccccc2-c2ccc(N(c3cccc(-c4ccccc4)c3)c3ccc4c5ccccc5n(-c5c(-c6ccccc6)cccc5-c5ccccc5)c4c3)cc21] | 2.32 | −5.10 | 125 |
| | C5 | | | |
| C6 | 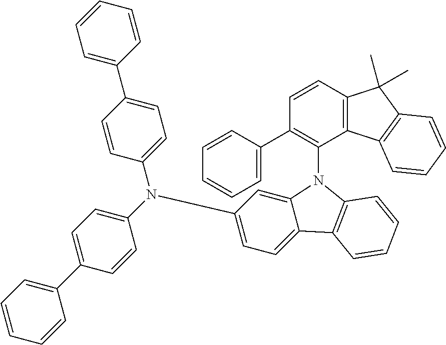 [SMILES: CC1(C)c2ccccc2-c2c1ccc(-c1ccccc1)c2-n1c2ccccc2c2ccc(N(c3ccc(-c4ccccc4)cc3)c3ccc(-c4ccccc4)cc3)cc21] | 2.78 | −5.07 | 130 |
| | C6 | | | |
| C7 | 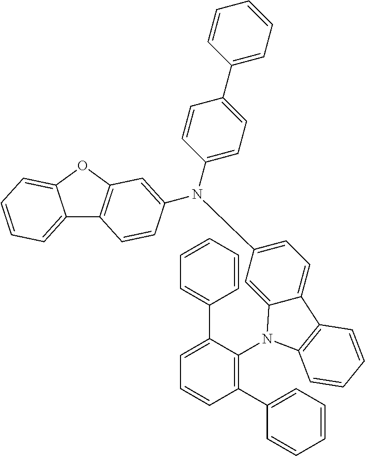 [SMILES: c1ccc(-c2ccc(N(c3ccc4c(c3)oc3ccccc34)c3ccc4c5ccccc5n(-c5c(-c6ccccc6)cccc5-c5ccccc5)c4c3)cc2)cc1] | 2.80 | −5.15 | 127 |
| | C7 | | | |
| C8 | 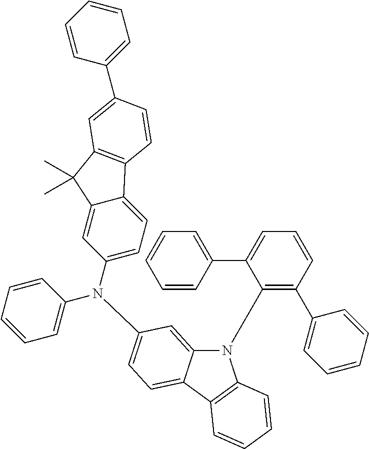 [SMILES: CC1(C)c2cc(-c3ccccc3)ccc2-c2ccc(N(c3ccccc3)c3ccc4c5ccccc5n(-c5c(-c6ccccc6)cccc5-c5ccccc5)c4c3)cc21] | 2.49 | −5.13 | 131 |
| | C8 | | | |
| C9 | 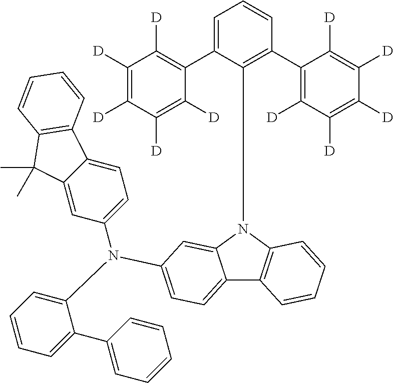 [SMILES: [2H]c1c([2H])c([2H])c(-c2cccc(-c3c([2H])c([2H])c([2H])c([2H])c3[2H])c2-n2c3ccccc3c3ccc(N(c4ccc5c(c4)C(C)(C)c4ccccc4-5)c4ccccc4-c4ccccc4)cc32)c([2H])c1[2H]] | 2.74 | −5.06 | 130 |
| | C9 | | | |
| C10 | 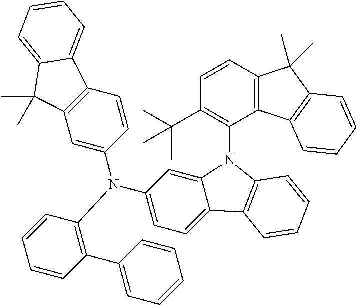 [SMILES: CC(C)(C)c1ccc2c(c1-n1c3ccccc3c3ccc(N(c4ccc5c(c4)C(C)(C)c4ccccc4-5)c4ccccc4-c4ccccc4)cc31)-c1ccccc1C2(C)C] | 2.84 | −5.11 | 128 |
| | C10 | | | |
| D1 | 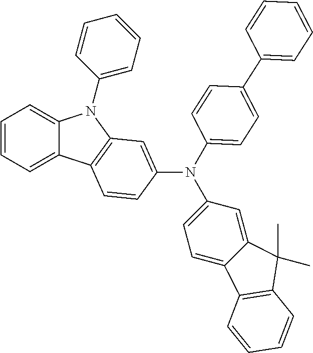 [SMILES: CC1(C)c2ccccc2-c2ccc(N(c3ccc(-c4ccccc4)cc3)c3ccc4c5ccccc5n(-c5ccccc5)c4c3)cc21] | 3.05 | −5.20 | 109 |
| | D1 | | | |
| D2 | 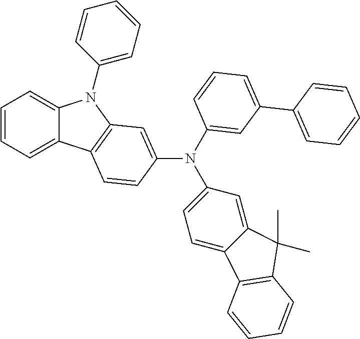 [SMILES: CC1(C)c2ccccc2-c2ccc(N(c3cccc(-c4ccccc4)c3)c3ccc4c5ccccc5n(-c5ccccc5)c4c3)cc21] | 3.11 | −5.23 | 107 |
| | D2 | | | |
| D3 | 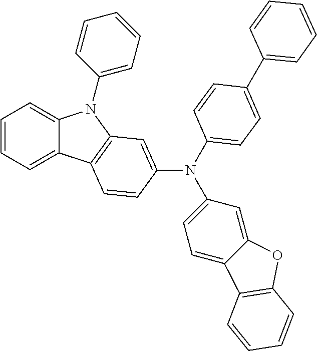 [SMILES: c1ccc(-c2ccc(N(c3ccc4c(c3)oc3ccccc34)c3ccc4c5ccccc5n(-c5ccccc5)c4c3)cc2)cc1] | 3.31 | −5.26 | 106 |
| | D3 | | | |
| D4 | 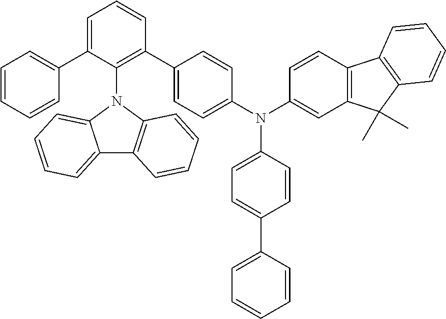 [SMILES: CC1(C)c2ccccc2-c2ccc(N(c3ccc(-c4ccccc4)cc3)c3ccc(-c4cccc(-c5ccccc5)c4-n4c5ccccc5c5ccccc54)cc3)cc21] | 3.20 | −5.19 | 105 |
| | D4 | | | |
| D5 | 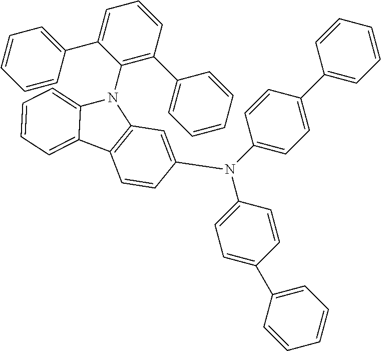 [SMILES: c1ccc(-c2ccc(N(c3ccc(-c4ccccc4)cc3)c3ccc4c5ccccc5n(-c5c(-c6ccccc6)cccc5-c5ccccc5)c4c3)cc2)cc1] | 3.27 | −5.19 | 123 |
| | D5 | | | |

According to the voltage data in Table 1, it could be seen that the compound containing the carbazole derivative as provided by the present invention has a lower voltage value, a higher HOMO energy level value and a higher glass transition temperature as compared with the compounds of the comparative examples. By comparison, it could be seen that in C2, C5, C7, C8, and C9 as compared with D1, D2, and D3, by respectively introducing two phenyl groups in the ortho positions of the phenyl group connected to carbazole, the phenyl groups in the ortho positions had a relatively strong electron-donating ability, the HOMO energy level value of the compound could be improved, the steric hindrance of the molecule could be significantly increased, the molecular rotation during heating was reduced, and the glass transition temperature of the molecule was increased. In C3 and C10 as compared with D1, D2, and D3, by introducing one phenyl group in an ortho position of the phenyl group connected to carbazole, the newly introduced phenyl group and the central benzene ring formed a fluorene structure, which could form a crisscross spatial structure, which was beneficial to the stacking between molecules and facilitated the transport of holes between molecules. In C1, C4 and C6 as compared with D1, D2 and D3, by introducing two phenyl groups in the ortho positions of the phenyl group connected to carbazole with one of the newly introduced phenyl groups and the central benzene ring forming a fluorene structure. The ortho positions of the phenyl group had a relatively strong electron-donating ability and the fluorene structure could form a crisscross spatial structure, which was beneficial to the stacking between molecules and facilitated the transport of holes between molecules. In C1-C10 as compared with D4, the diarylamine was connected to the benzene rings of the carbazole, whereby the conjugation of two benzene rings in the carbazole was better, which could increase the hole transport rate, and the carbazole had a relatively strong electron-donating ability, which could significantly improve the HOMO energy level value of the compound. Compared with D5, C1, C2, C4, C5, C6, C7, C8, and C9 had fluorene structures, in which the included angle between the planes of the two benzene rings was smaller and the conjugation between the two benzene rings was better, which was beneficial to delocalization of electrons in the molecule and could effectively reduce the hole transport barrier potential and increase the hole transport rate, so the compound containing the carbazole derivative as provided by the present invention had a lower voltage value.

### DEVICE EXAMPLE

The organic electroluminescent devices of the following examples comprised an anode 2, a hole transport region, a luminescent layer 5, an electron transport region, and a cathode 8, which were sequentially arranged on a substrate 1, wherein the hole transport region comprised a hole transport layer 3 and a luminescent auxiliary layer 4; the electron transport region comprised an electron transport layer 6 and an electron injection layer 7; and the luminescent layer 5 was composed of a host and a dopant guest, wherein the host of the luminescent layer could be composed of one molecular material or a plurality of molecular materials. The typical structure of the organic electroluminescent devices was as shown in FIG. 1.

For the anodes of the following examples, anode materials commonly used in the art, such as ITO, Ag or multilayer structures thereof, were used. For the hole injection unit, hole injection materials commonly used in the art were used, and at the same time F4TCNQ, HATCN, NDP-9, etc. were added for doping. For the light-emitting unit, luminescent materials commonly used in the art were used, for example, they could be composed of a host material doped with an emitting guest material, wherein the emitting guest material could be an organic material such as a pyrene compound or could also be a metal complex (such as the metal Ir or Pt). For the electron transport unit, electron transport materials commonly used in the art were used. For the electron injection layer, electron injection materials commonly used in the art, such as Liq, LiF and Yb were used. For the cathode, materials commonly used in the art are used, such as the metals A1 and Ag or metal mixtures (Ag-doped Mg, Ag-doped Ca, etc.).

The electrode preparation method and the deposition method for each functional layer in the following examples were both conventional methods in the art, e.g., vacuum thermal evaporation or ink-jet printing. No more repetition would be given here, and only some process details and test methods in the preparation process were supplemented as follows.

### Device Example 1

This example provided a blue-light organic electroluminescent device, the preparation method of which was as follows. firstly, on an ITO layer (anode) formed on a substrate, C1 and F4TCNQ (at a mass ratio of C1 to F4TCNQ of 97:3) were deposited in vacuo with a thickness of 10 nm to form a hole injection layer; secondly, on the above hole injection layer, C1 was deposited in vacuo with a thickness of 120 nm to form a hole transport layer; thirdly, on the above hole transport layer, B Prime was deposited in vacuo with a thickness of 10 nm to form a luminescent auxiliary layer; again, on the above luminescent auxiliary layer, a mixture of BH and BD was deposited in vacuo with a thickness of 20 nm to form a luminescent layer, wherein BH was used as a host, BD was used as a dopant, and the mass ratio of the host to the dopant was 98:2; subsequently, on the above luminescent layer, a mixture of ET-01 and Liq (at a mass ratio of ET-01 to Liq of 1:1) was deposited in vacuo with a thickness of 35 nm to form an electron transport layer; then, on the above electron transport layer, LiF was deposited with a thickness of 0.2 nm to form an electron injection layer; and finally, on the above electron injection layer, aluminum (Al) was deposited with a thickness of 150 nm to form a cathode, thereby preparing a blue-light organic electroluminescent device. The molecular structural formulas of the materials of the layers other than the hole transport layer were as follows:

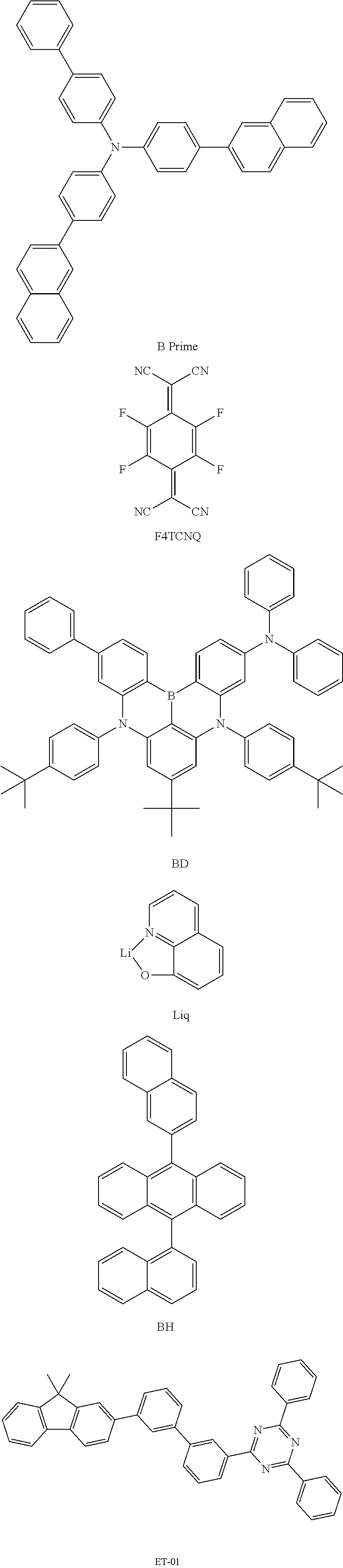

### Blue-light Device Examples 2-10

This example provided a blue-light organic electroluminescent device, the preparation method of which was as follows. Compound C1 used in the hole injection layer and hole transport layer in Blue-light Device Example 1 was replaced by Compound C2, C3, C4, C5, C6, C7, C8, C9 or C10, and the other preparation steps were the same as in Blue-light Device Example 1, so as to separately prepare a blue-light organic electroluminescent device.

### Comparative Blue-light Device Examples 1-5

This comparative example provided a blue-light organic electroluminescent device which had been experimented in the research process, the preparation method of which was as follows. Compound C1 used in the hole injection layer and hole transport layer in Blue-light Device Example 1 was replaced by Comparative Compound D1, D2, D3, D4, or D5, and the other preparation steps were the same as in Blue-light Device Example 1, so as to separately prepare a blue-light organic electroluminescent device.

### Effect Example 2

The organic electroluminescent devices provided by Blue-light Device Examples 1 to 10 and Comparative Blue-light Device Examples 1 to 5 were tested by standard methods. The organic electroluminescent device was measured at a current density of J=10 mA/cm² for the driving voltage, brightness, electroluminescent current efficiency (measured as cd/A), and external quantum efficiency (EQE, measured in percentage), which were calculated, as a function of luminous density, from a current/voltage/luminous density characteristic curve (IVL characteristic curve) showing Lambertian emission characteristics, for luminous spectrum.

The lifetime LT was defined as the time for the brightness to decrease from the initial luminous brightness L₀ to a specific proportion L₁, during working at a constant current J; The expressions J=50 mA/cm² and L₁=90% meant that during working at 50 mA/cm², the luminous brightness decreased to 90% of the initial value L₀ thereof after the time LT. Similarly, the expressions J=20 mA/cm² and L₁=80% meant that during working at 20 mA/cm², the luminous brightness decreased to 80% of the initial value L₀ thereof after the time LT.

The organic electroluminescent devices provided by Blue-light Device Examples 1 to 10 and Comparative Blue-light Device Examples 1 to 5 were respectively tested for performance. The specific test instruments and methods were as follows: the brightness was tested by means of spectrum scanner PhotoResearch PR-635; the current density and turn-on voltage were tested by digital SourceMeter Keithley 2400; and the lifetime test was carried out by means of LT-96ch lifetime test device. The specific test results were as shown in Table 2.

TABLE 2
Performance test results of blue-light devices

| Hole | | | | @J = 10 | |
|---|---|---|---|---|---|
| transport | | Vop | EQE | mA/cm² | Light |
| material | Structure of compound | (V) | (%) | LT95 (h) | color |
| C1 | 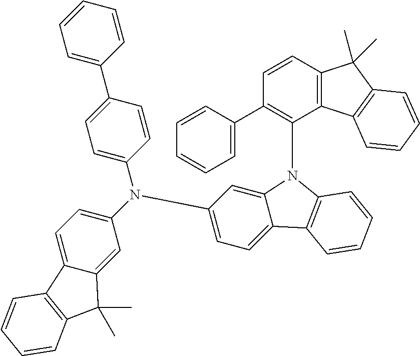 [SMILES: CC1(C)c2ccccc2-c2ccc(N(c3ccc(-c4ccccc4)cc3)c3ccc4c5ccccc5n(-c5c(-c6ccccc6)ccc6c5-c5ccccc5C6(C)C)c4c3)cc21] | 3.91 | 6.51 | 120.38 | Blue light |
| | C1 | | | | |
| C2 | 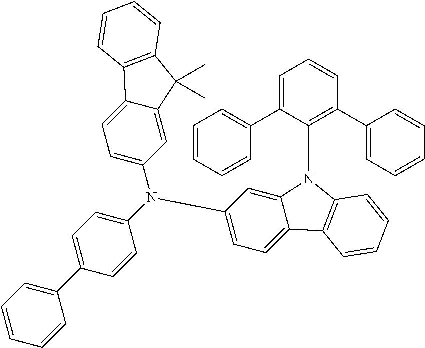 [SMILES: CC1(C)c2ccccc2-c2ccc(N(c3ccc(-c4ccccc4)cc3)c3ccc4c5ccccc5n(-c5c(-c6ccccc6)cccc5-c5ccccc5)c4c3)cc21] | 4.00 | 6.74 | 114.92 | Blue light |
| | C2 | | | | |
| C3 | 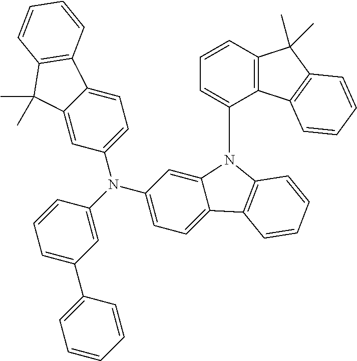 [SMILES: CC1(C)c2ccccc2-c2ccc(N(c3cccc(-c4ccccc4)c3)c3ccc4c5ccccc5n(-c5cccc6c5-c5ccccc5C6(C)C)c4c3)cc21] | 3.98 | 6.78 | 111.94 | Blue light |
| | C3 | | | | |
| C4 | 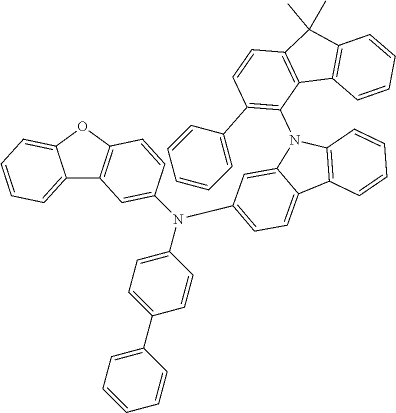 [SMILES: CC1(C)c2ccccc2-c2c1ccc(-c1ccccc1)c2-n1c2ccccc2c2ccc(N(c3ccc(-c4ccccc4)cc3)c3ccc4oc5ccccc5c4c3)cc21] | 3.96 | 6.54 | 158.45 | Blue light |
| | C4 | | | | |
| C5 | 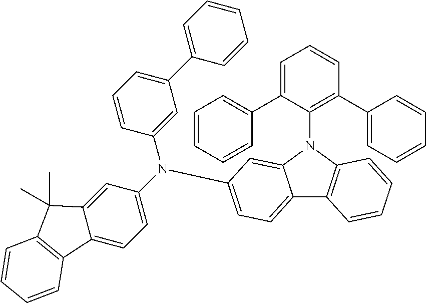 [SMILES: CC1(C)c2ccccc2-c2ccc(N(c3cccc(-c4ccccc4)c3)c3ccc4c5ccccc5n(-c5c(-c6ccccc6)cccc5-c5ccccc5)c4c3)cc21] | 4.10 | 6.90 | 115.95 | Blue light |
| | C5 | | | | |
| C6 | 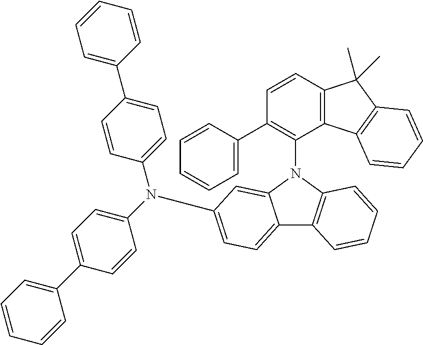 [SMILES: CC1(C)c2ccccc2-c2c1ccc(-c1ccccc1)c2-n1c2ccccc2c2ccc(N(c3ccc(-c4ccccc4)cc3)c3ccc(-c4ccccc4)cc3)cc21] | 4.03 | 6.68 | 114.23 | Blue light |
| | C6 | | | | |
| C7 | 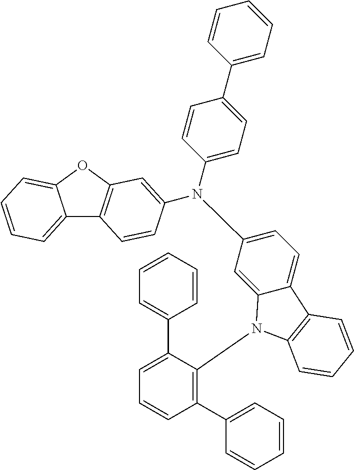 [SMILES: c1ccc(-c2ccc(N(c3ccc4c(c3)oc3ccccc34)c3ccc4c5ccccc5n(-c5c(-c6ccccc6)cccc5-c5ccccc5)c4c3)cc2)cc1] | 3.95 | 6.91 | 131.88 | Blue light |
| | C7 | | | | |
| C8 | 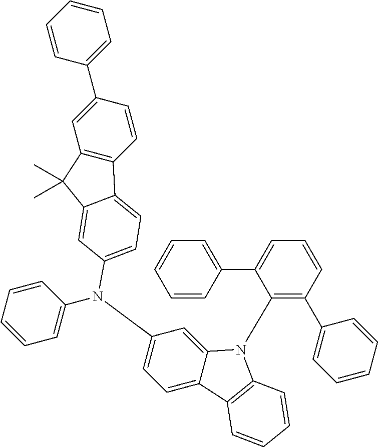 [SMILES: CC1(C)c2cc(-c3ccccc3)ccc2-c2ccc(N(c3ccccc3)c3ccc4c5ccccc5n(-c5c(-c6ccccc6)cccc5-c5ccccc5)c4c3)cc21] | 4.08 | 7.02 | 117.74 | Blue light |
| | C8 | | | | |
| C9 | 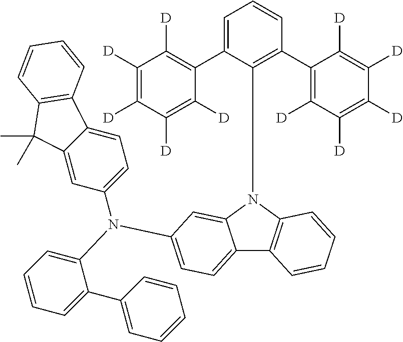 [SMILES: [2H]c1c([2H])c([2H])c(-c2cccc(-c3c([2H])c([2H])c([2H])c([2H])c3[2H])c2-n2c3ccccc3c3ccc(N(c4ccc5c(c4)C(C)(C)c4ccccc4-5)c4ccccc4-c4ccccc4)cc32)c([2H])c1[2H]] | 4.06 | 6.79 | 120.09 | Blue light |
| | C9 | | | | |
| C10 | 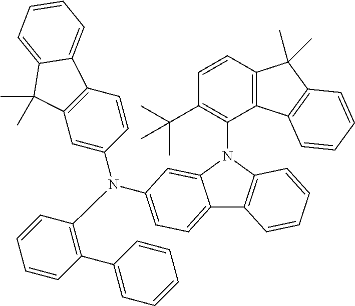 [SMILES: CC(C)(C)c1ccc2c(c1-n1c3ccccc3c3ccc(N(c4ccc5c(c4)C(C)(C)c4ccccc4-5)c4ccccc4-c4ccccc4)cc31)-c1ccccc1C2(C)C] | 4.12 | 7.12 | 131.02 | Blue light |
| | C10 | | | | |
| D1 | 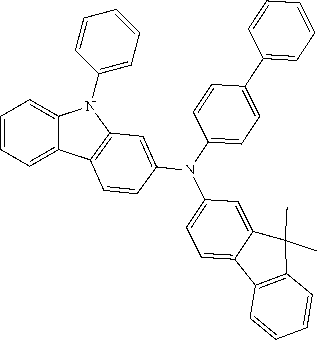 [SMILES: CC1(C)c2ccccc2-c2ccc(N(c3ccc(-c4ccccc4)cc3)c3ccc4c5ccccc5n(-c5ccccc5)c4c3)cc21] | 4.26 | 5.05 | 79.19 | Blue light |
| | D1 | | | | |
| D2 | 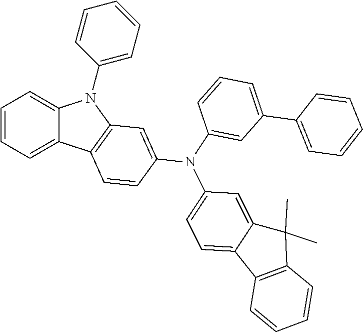 [SMILES: CC1(C)c2ccccc2-c2ccc(N(c3cccc(-c4ccccc4)c3)c3ccc4c5ccccc5n(-c5ccccc5)c4c3)cc21] | 4.29 | 5.28 | 84.36 | Blue light |
| | D2 | | | | |
| D3 | 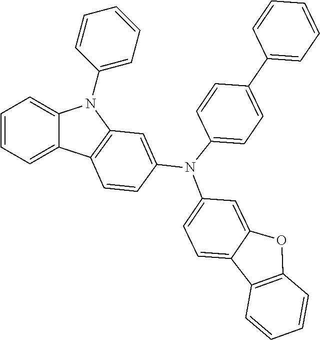 [SMILES: c1ccc(-c2ccc(N(c3ccc4c(c3)oc3ccccc34)c3ccc4c5ccccc5n(-c5ccccc5)c4c3)cc2)cc1] | 4.25 | 5.24 | 65.38 | Blue light |
| | D3 | | | | |
| D4 | 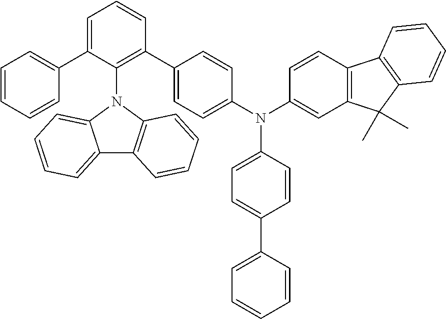 [SMILES: CC1(C)c2ccccc2-c2ccc(N(c3ccc(-c4ccccc4)cc3)c3ccc(-c4cccc(-c5ccccc5)c4-n4c5ccccc5c5ccccc54)cc3)cc21] | 4.29 | 5.13 | 70.90 | Blue light |
| | D4 | | | | |
| D5 | 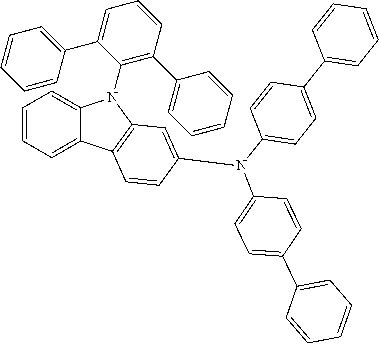 [SMILES: c1ccc(-c2ccc(N(c3ccc(-c4ccccc4)cc3)c3ccc4c5ccccc5n(-c5c(-c6ccccc6)cccc5-c5ccccc5)c4c3)cc2)cc1] | 4.31 | 5.22 | 92.13 | Blue light |
| | D5 | | | | |

According to the device performance test results in Table 2, it could be seen that compared with the comparative compounds, the use of the compound containing the carbazole derivative as provided by the present invention as the hole transport layer material could improve the luminous efficiency of the organic electroluminescent device, reduce the driving voltage of the organic electroluminescent device, and prolong the lifetime of the organic electroluminescent device, because the compound containing the carbazole derivative as provided by the present invention had a higher hole transport rate, a higher HOMO energy level value and a higher glass transition temperature as compared with the compounds of the comparative examples. Where the compound containing the carbazole derivative as provided by the present invention was applied to the hole transport layer of the organic electroluminescent device, the hole transport layer had a higher HOMO energy level value, so that the hole transport barrier potential could be reduced, and in turn, the driving voltage of the organic electroluminescent device could be reduced. The hole transport layer had a relatively high hole transport rate, which could effectively improve the hole injection and migration efficiency in the organic electroluminescent device, thus ensuring that the organic electroluminescent device attained the excellent effects of a high luminous efficiency and a low starting voltage. The hole transport layer had a higher glass transition temperature, and the glass transition temperature (Tg) of the material was associated with the film-forming property and thermal stability of the material. The higher the glass transition temperature of the material, the better the thermal stability of the material, and the material was less prone to crystallization or agglomeration, thus prolonging the lifetime of the organic electroluminescent device. In summary, the compound containing the carbazole derivative as provided by the present invention was a hole transport layer material with excellent performance and had a wide application prospect and economic benefits.

The above description is only preferred embodiments of the present invention, and the scope of protection of the present invention is not limited thereto. Any changes, substitutions, etc. readily conceivable to any of those familiar with the technical field within the technical scope of the disclosure of the present invention should be included in the scope of protection of the present invention. Therefore, for the scope of protection of the present invention, the scope of protection of the claims shall prevail.

1. A compound containing a carbazole derivative, characterized in that the general structural formula of the compound containing the carbazole derivative is one of the following structures: 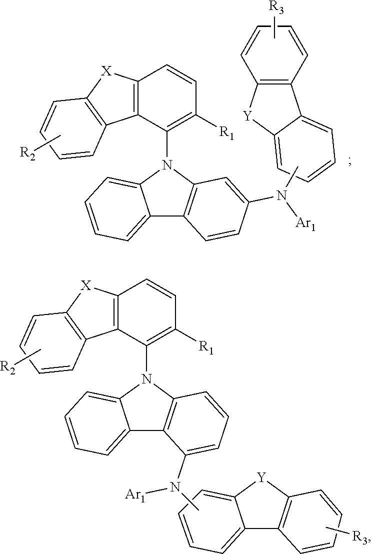
wherein
R₁ is selected from any one of hydrogen, deuterated or non-deuterated phenyl, and deuterated or non-deuterated alkyl with a carbon atom number of 1 to 5;
R₂ represents hydrogen on a benzene ring connected thereto, wherein any hydrogen can be independently replaced by deuterium;
R₃ represents hydrogen on a benzene ring connected thereto, wherein any hydrogen can be independently replaced by any one of deuterium, deuterated or non-deuterated phenyl, deuterated or non-deuterated alkyl with a carbon atom number of 1 to 5, and deuterated or non-deuterated alkenyl with a carbon atom number of 2 to 5;
X and Y are each independently selected from any one of non-bonding, 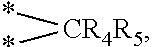
O, and S, and X and Y are not both selected from non-bonding;
Ar₁ is selected from any one of or a combination of arbitrarily some of substituted or unsubstituted aryl with a carbon atom number of 6 to 30, substituted or unsubstituted fused cyclic aryl with a carbon atom number of 10 to 30, and substituted or unsubstituted fused cyclic heteroaryl with a carbon atom number of 9 to 30;
R₄ and R₅ are each independently selected from any one of or a combination of arbitrarily some of hydrogen, deuterium, substituted or unsubstituted alkyl with a carbon atom number of 1 to 10, substituted or unsubstituted cycloalkyl with a carbon atom number of 3 to 15, and substituted or unsubstituted aryl with a carbon atom number of 6 to 15;
when R₄, R₅, and Ar₁ have substituents, the substituents on R₄, R₅, and Ar₁ are each independently selected from any one of or a combination of arbitrarily some of deuterium, alkyl with a carbon atom number of 1 to 10, aryl with a carbon atom number of 6 to 30, heteroaryl with a carbon atom number of 5 to 30, fused cyclic aryl with a carbon atom number of 10 to 30, fused cyclic heteroaryl with a carbon atom number of 9 to 30, and cycloalkyl with a carbon atom number of 3 to 10; and the above two or more adjacent substituents can be bonded to one another via a linker group or a single bond to form an aliphatic ring with a carbon atom number of 6 to 15 or an aromatic ring with a carbon atom number of 6 to 30; and
any hydrogen on the compound containing the carbazole derivative can be independently replaced by deuterium, alkyl, or cycloalkyl.

2. The compound containing the carbazole derivative according to claim 1, characterized in that the structure of the compound containing the carbazole derivative is selected from any one of the following structures:

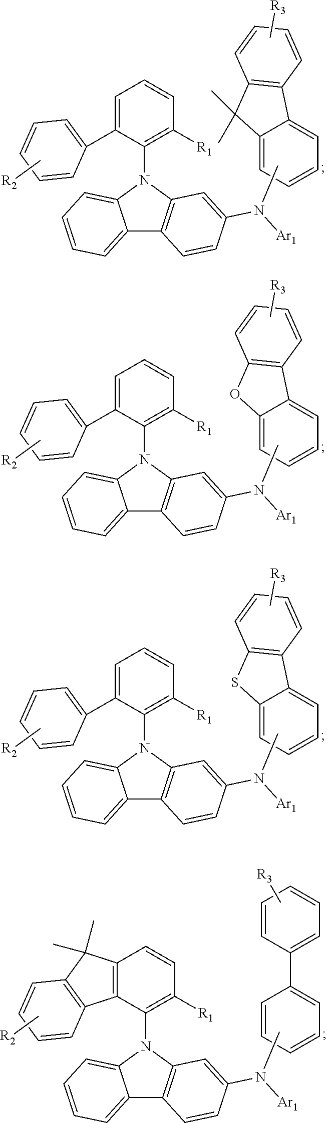

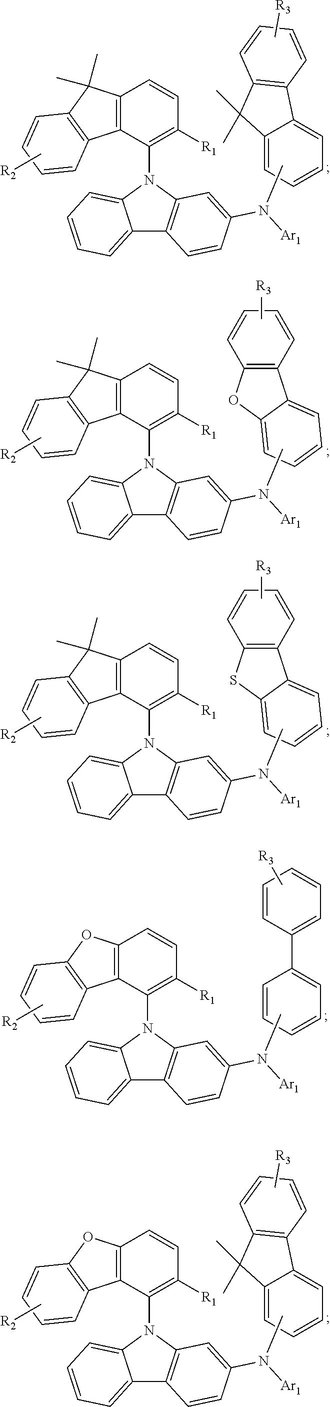

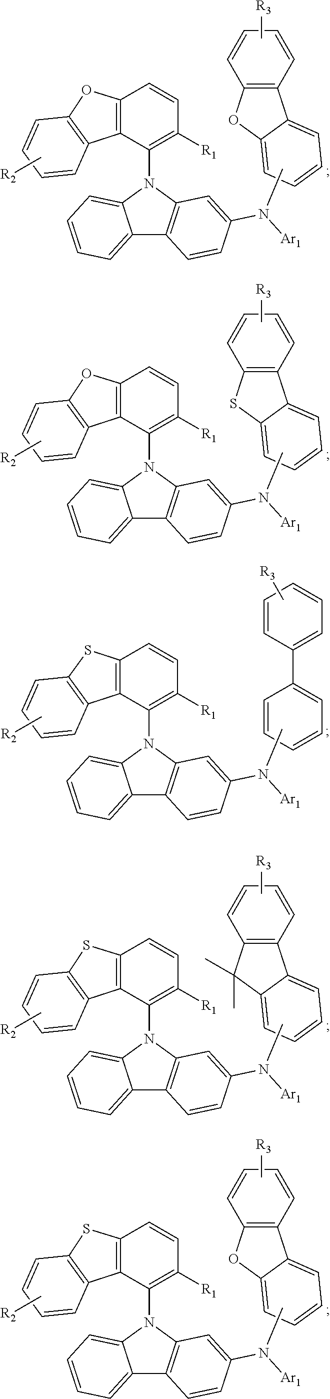

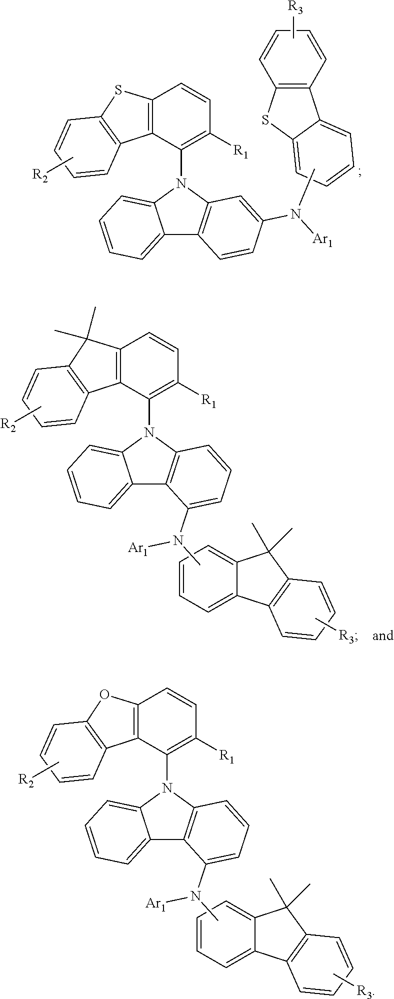

3. The compound containing the carbazole derivative according to claim 2, characterized in that the structure of the compound containing the carbazole derivative is selected from any one of the following structures:

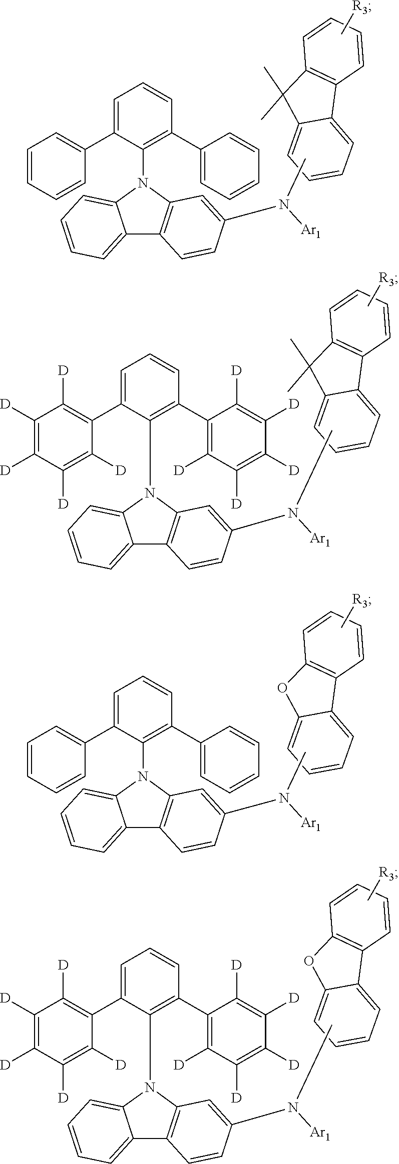

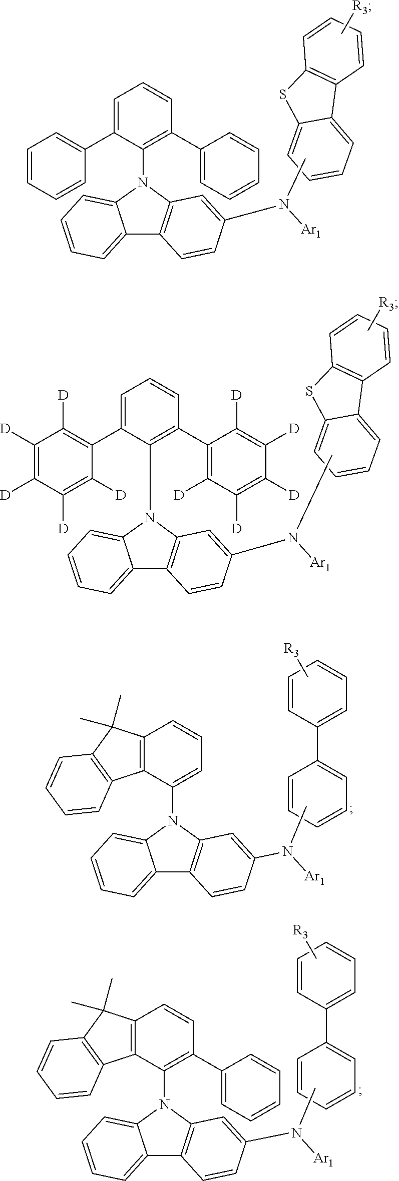

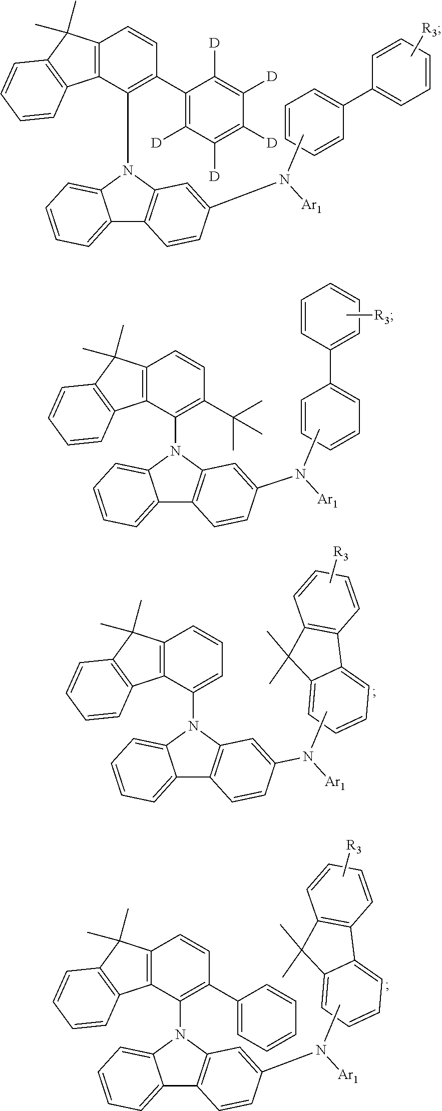

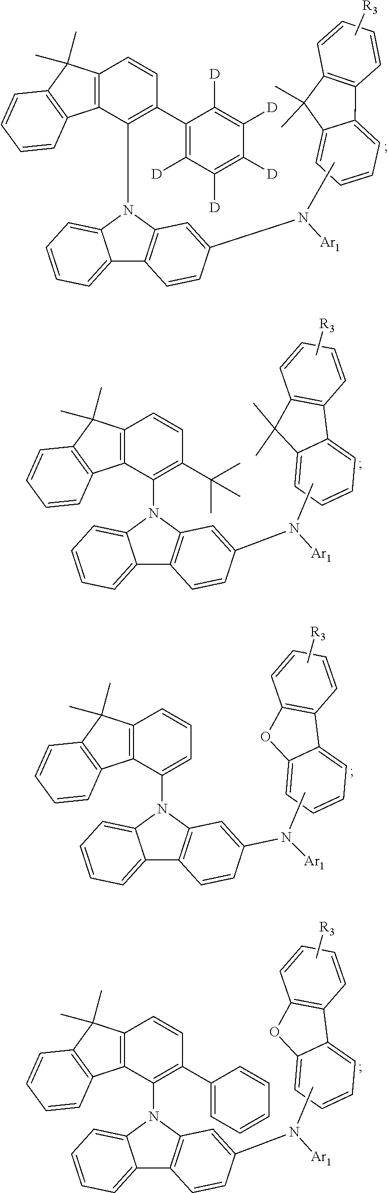

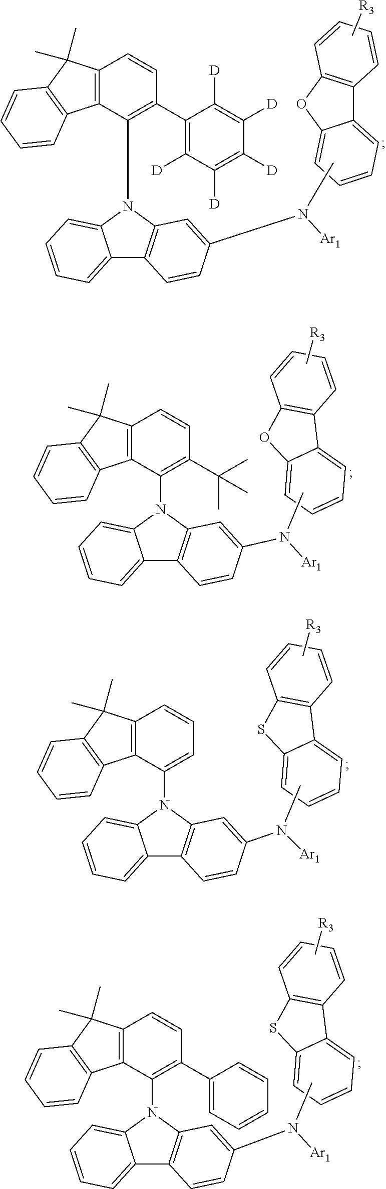

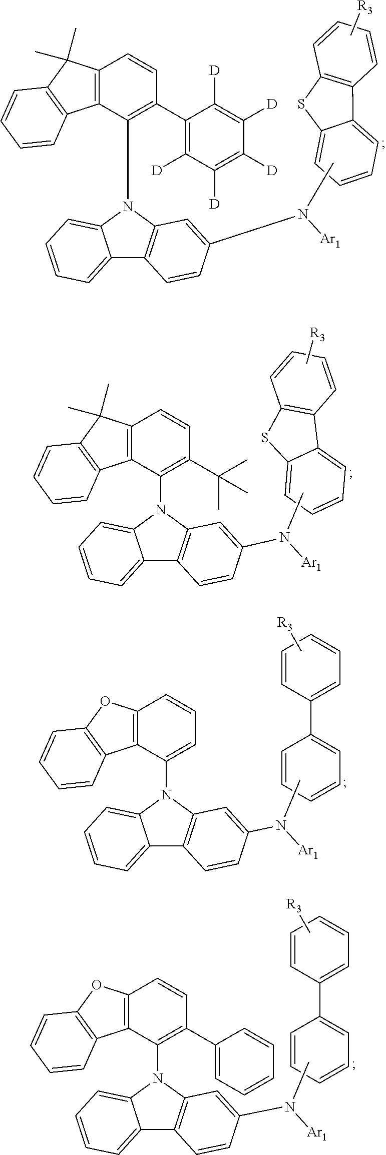

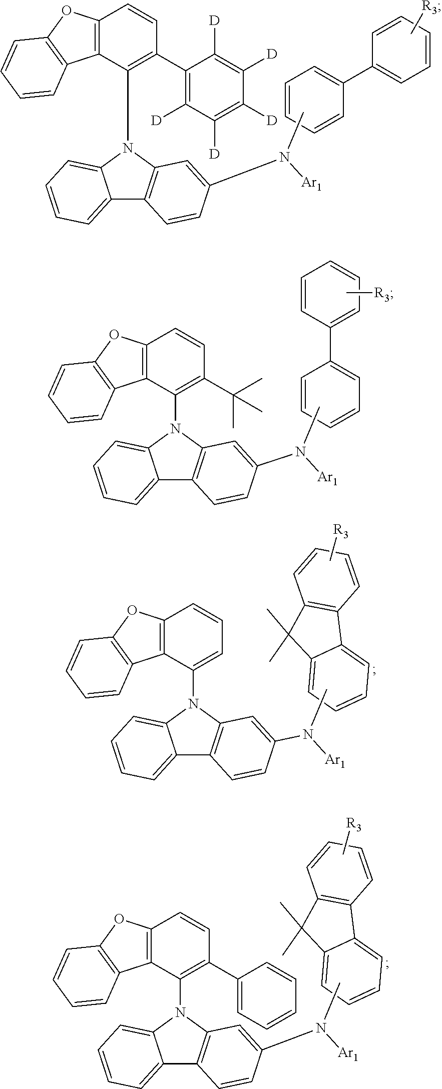

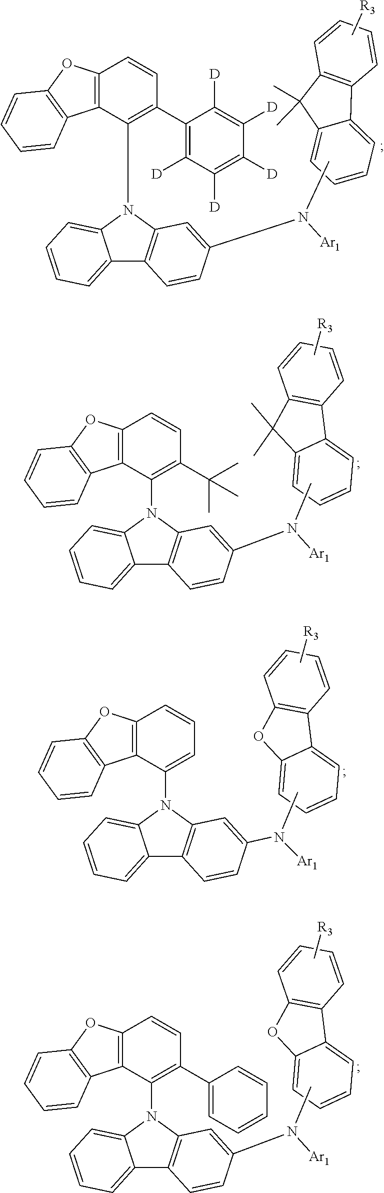

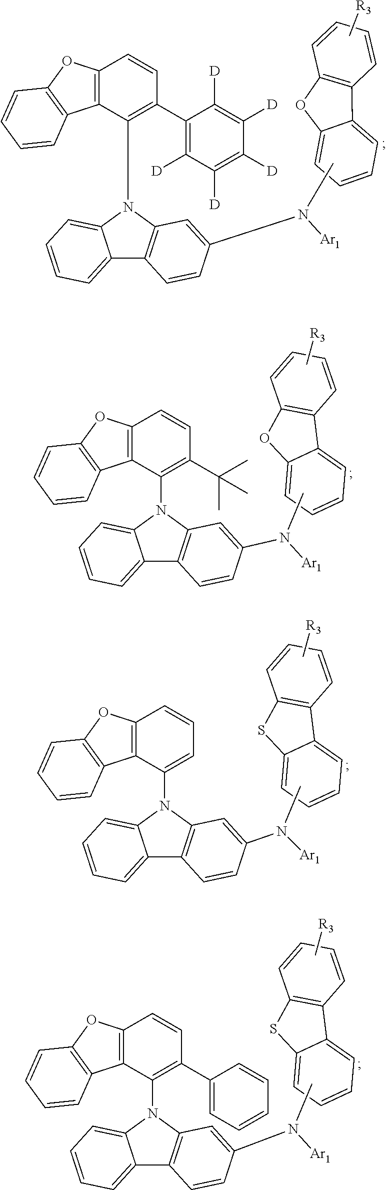

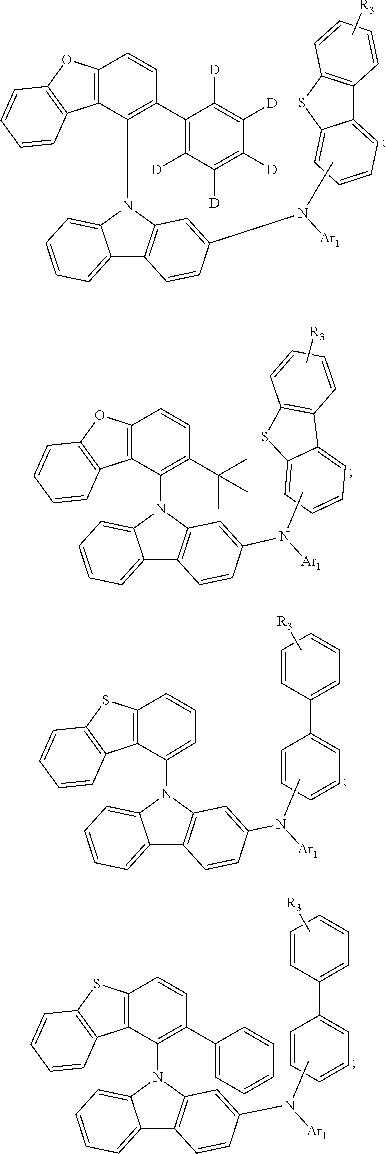

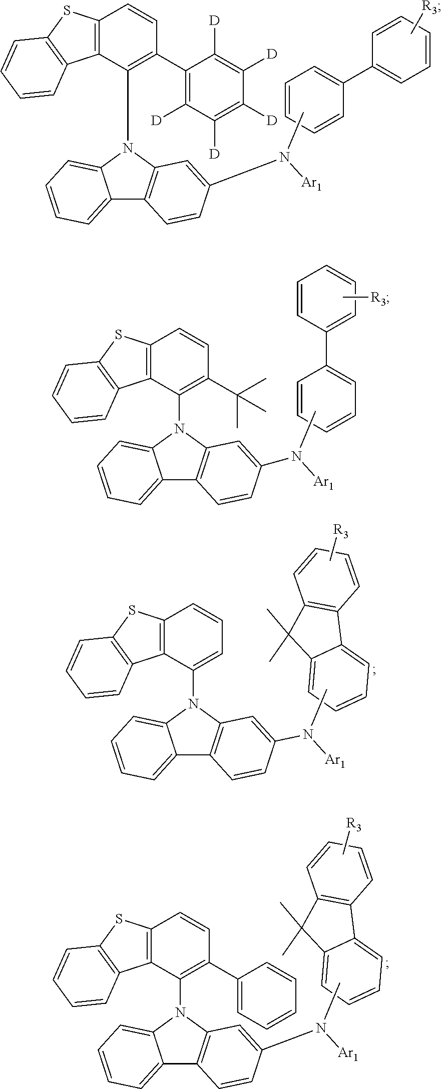

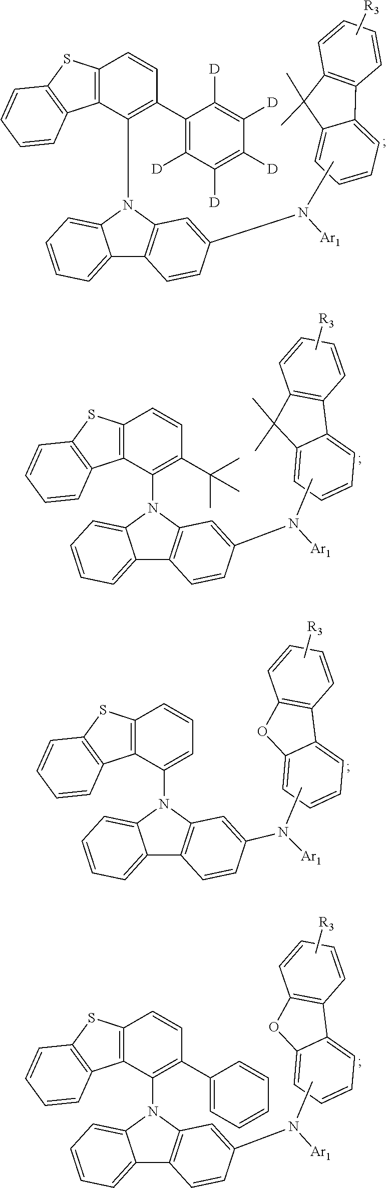

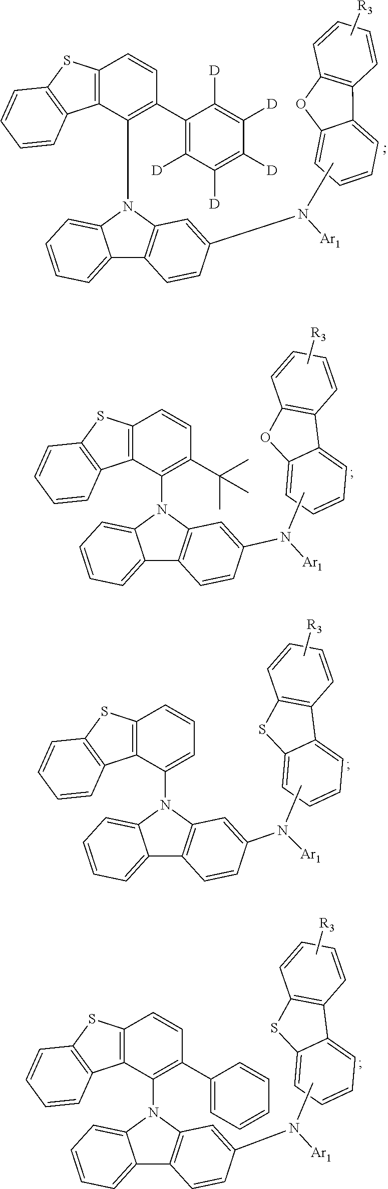

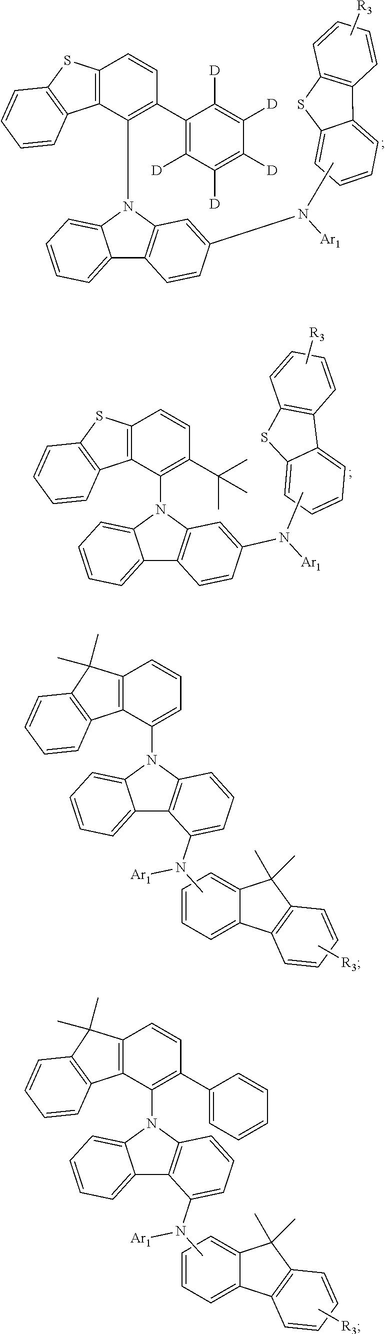

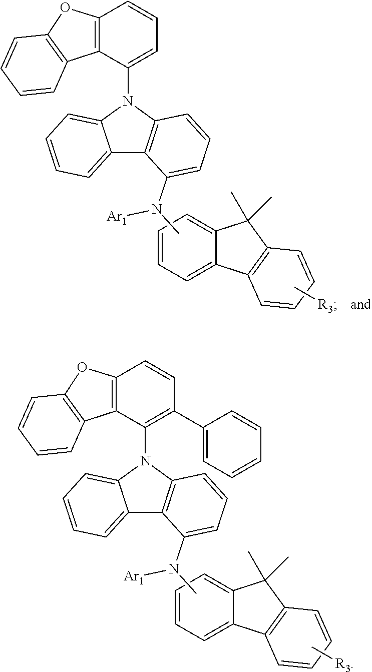

4. The compound containing the carbazole derivative according to claim 3, characterized in that the structure of the compound containing the carbazole derivative is selected from any one of the following structures:

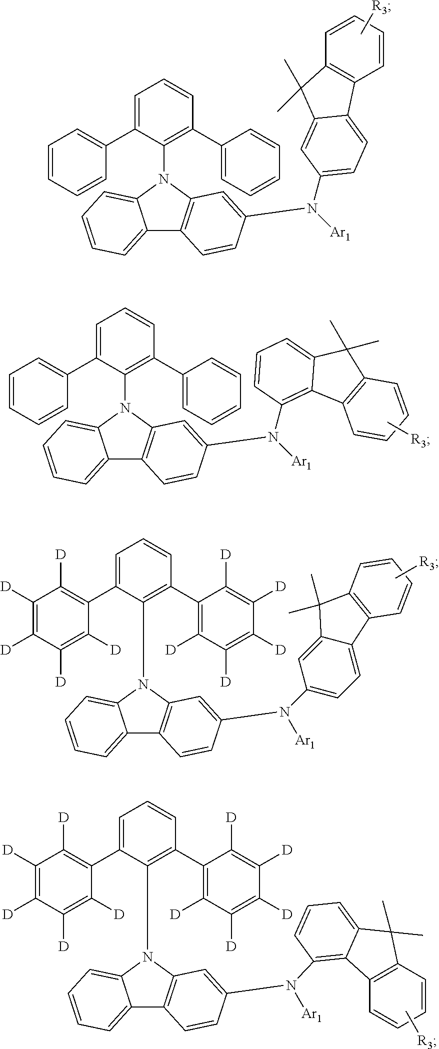

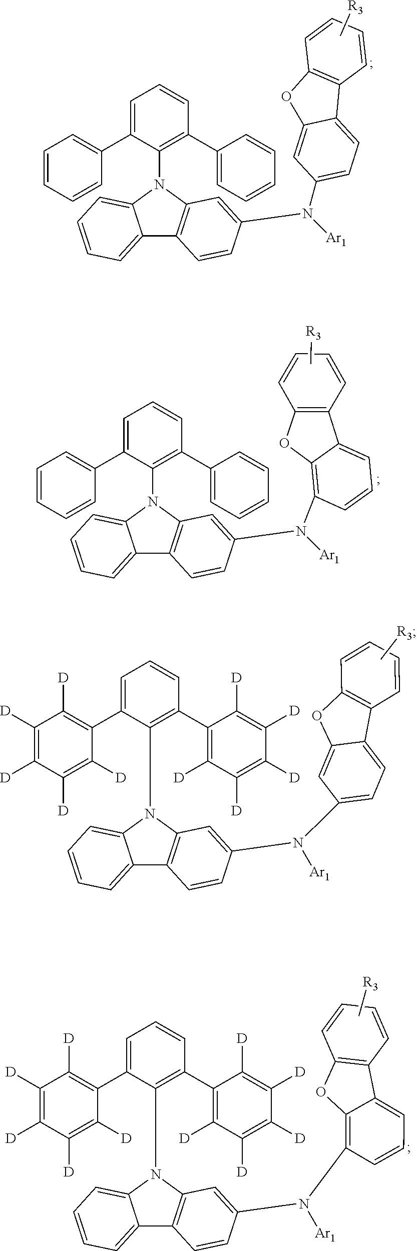

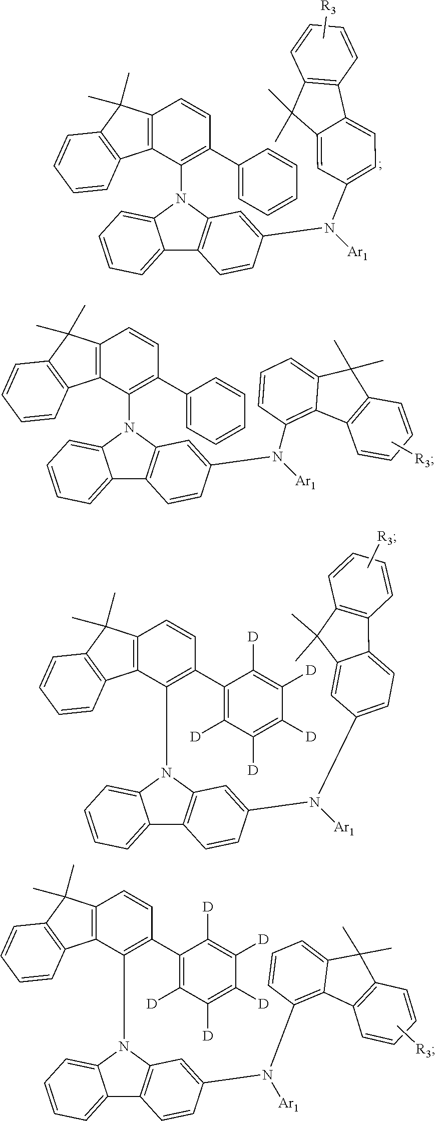

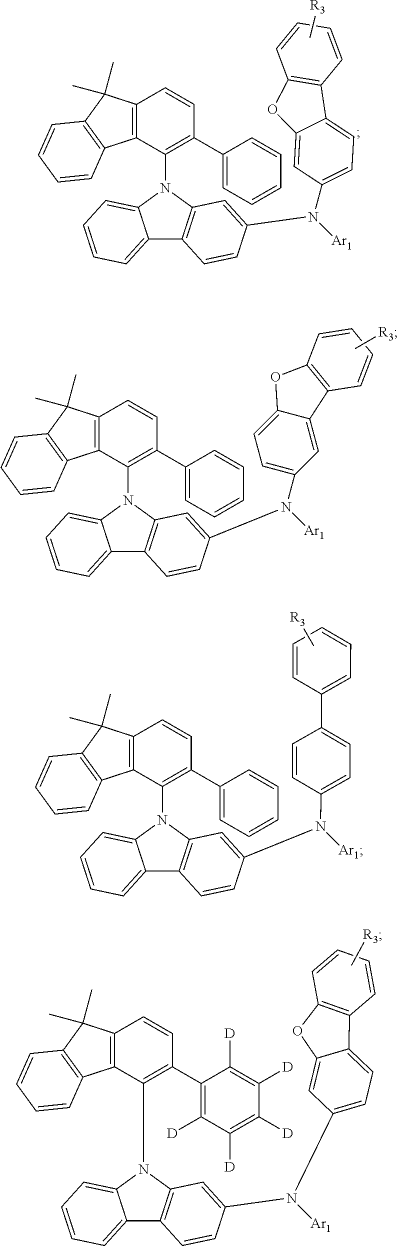

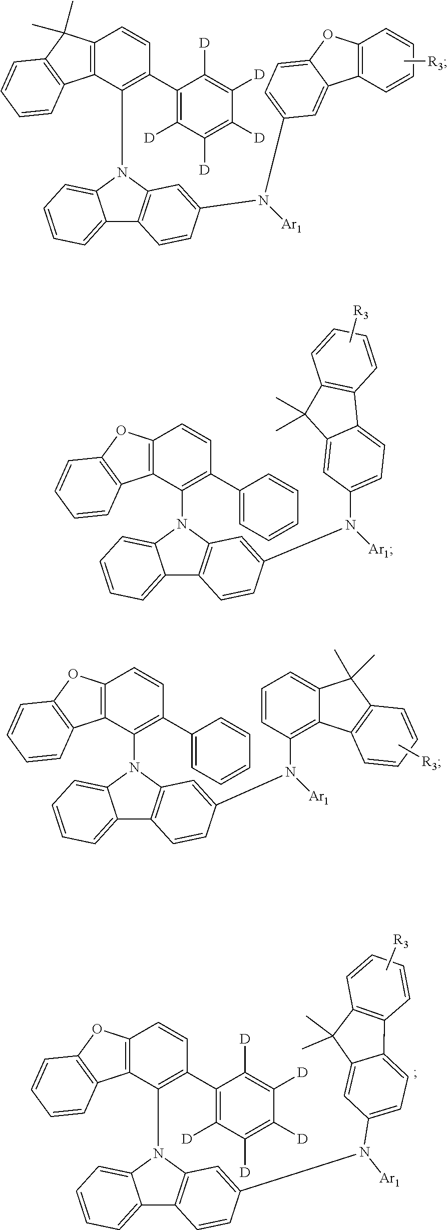

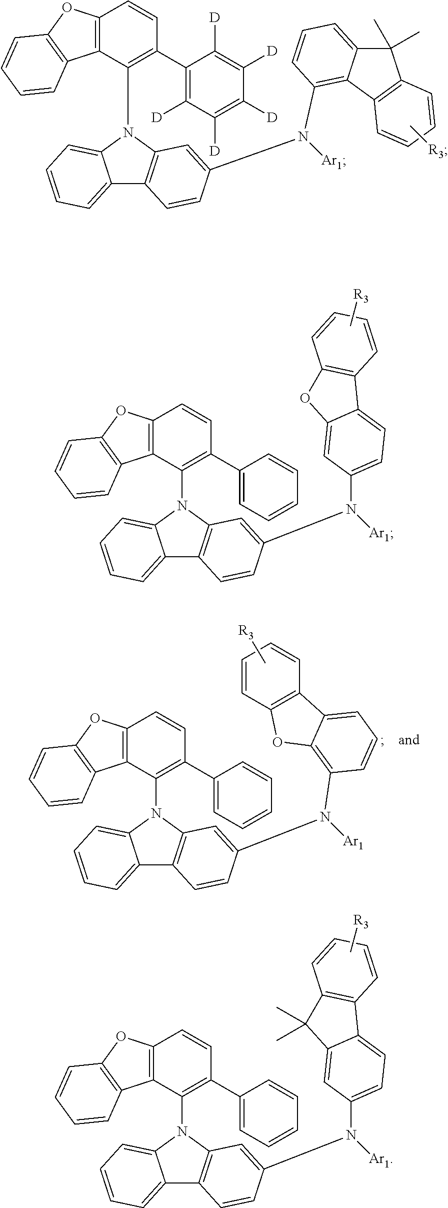

5. The compound containing the carbazole derivative according to claim 1, characterized in that Ar1 is selected from any one of substituted or unsubstituted aryl with a carbon atom number of 6 to 20, substituted or unsubstituted naphthyl, substituted or unsubstituted phenanthryl, substituted or unsubstituted 9,9-dimethylfluorenyl, and deuterated or non-deuterated phenyldibenzofuranyl.

6. The compound containing the carbazole derivative according to claim 1, characterized in that the compound containing the carbazole derivative is selected from any one of the following compounds:

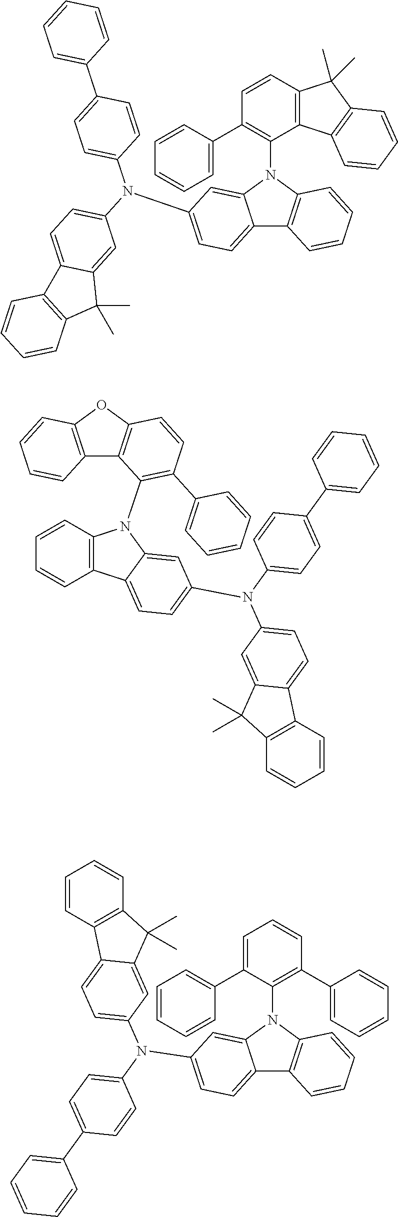 [SMILES: CC1(C)c2ccccc2-c2ccc(N(c3ccc(-c4ccccc4)cc3)c3ccc4c5ccccc5n(-c5c(-c6ccccc6)ccc6c5-c5ccccc5C6(C)C)c4c3)cc21 ; CC1(C)c2ccccc2-c2ccc(N(c3ccc(-c4ccccc4)cc3)c3ccc4c5ccccc5n(-c5c(-c6ccccc6)ccc6oc7ccccc7c56)c4c3)cc21 ; CC1(C)c2ccccc2-c2ccc(N(c3ccc(-c4ccccc4)cc3)c3ccc4c5ccccc5n(-c5c(-c6ccccc6)cccc5-c5ccccc5)c4c3)cc21]

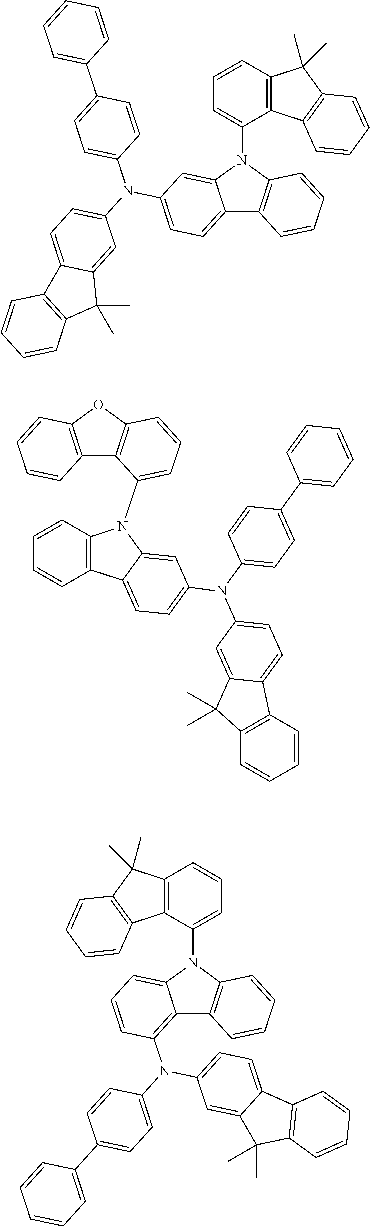 [SMILES: CC1(C)c2ccccc2-c2ccc(N(c3ccc(-c4ccccc4)cc3)c3ccc4c5ccccc5n(-c5cccc6c5-c5ccccc5C6(C)C)c4c3)cc21 ; CC1(C)c2ccccc2-c2ccc(N(c3ccc(-c4ccccc4)cc3)c3ccc4c5ccccc5n(-c5cccc6oc7ccccc7c56)c4c3)cc21 ; CC1(C)c2ccccc2-c2ccc(N(c3ccc(-c4ccccc4)cc3)c3cccc4c3c3ccccc3n4-c3cccc4c3-c3ccccc3C4(C)C)cc21]

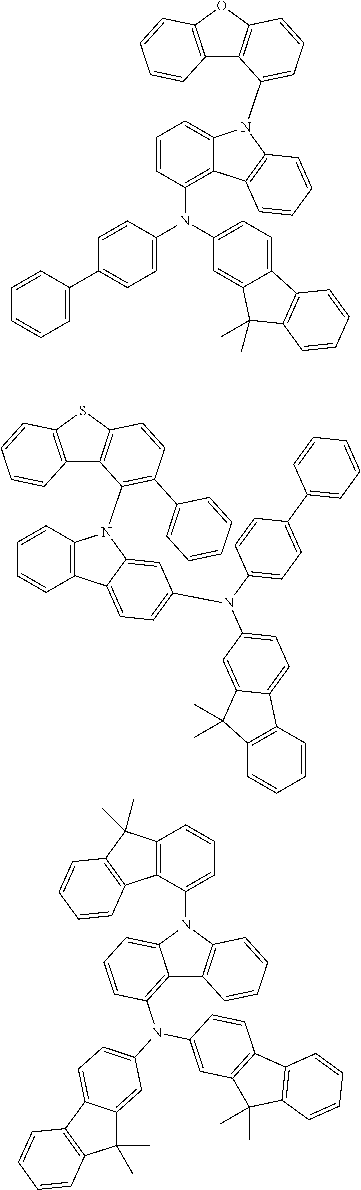 [SMILES: CC1(C)c2ccccc2-c2ccc(N(c3ccc(-c4ccccc4)cc3)c3cccc4c3c3ccccc3n4-c3cccc4oc5ccccc5c34)cc21 ; CC1(C)c2ccccc2-c2ccc(N(c3ccc(-c4ccccc4)cc3)c3ccc4c5ccccc5n(-c5c(-c6ccccc6)ccc6sc7ccccc7c56)c4c3)cc21 ; CC1(C)c2ccccc2-c2ccc(N(c3ccc4c(c3)C(C)(C)c3ccccc3-4)c3cccc4c3c3ccccc3n4-c3cccc4c3-c3ccccc3C4(C)C)cc21]

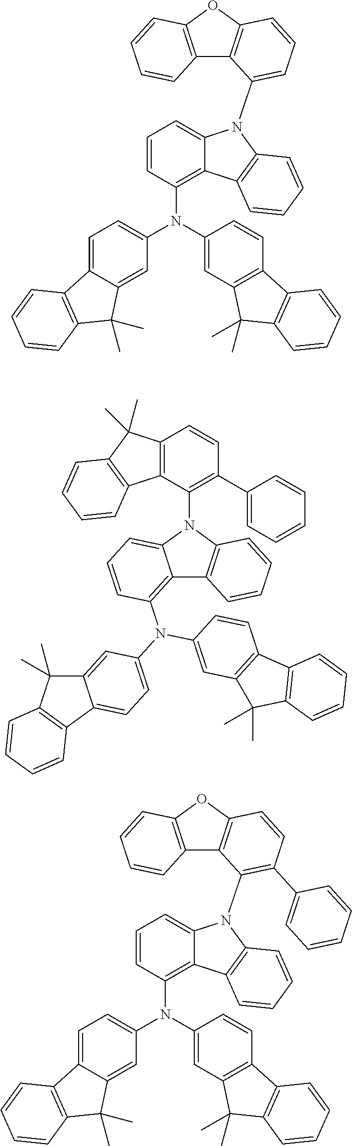 [SMILES: CC1(C)c2ccccc2-c2ccc(N(c3ccc4c(c3)C(C)(C)c3ccccc3-4)c3cccc4c3c3ccccc3n4-c3cccc4oc5ccccc5c34)cc21 ; CC1(C)c2ccccc2-c2ccc(N(c3ccc4c(c3)C(C)(C)c3ccccc3-4)c3cccc4c3c3ccccc3n4-c3c(-c4ccccc4)ccc4c3-c3ccccc3C4(C)C)cc21 ; CC1(C)c2ccccc2-c2ccc(N(c3ccc4c(c3)C(C)(C)c3ccccc3-4)c3cccc4c3c3ccccc3n4-c3c(-c4ccccc4)ccc4oc5ccccc5c34)cc21]

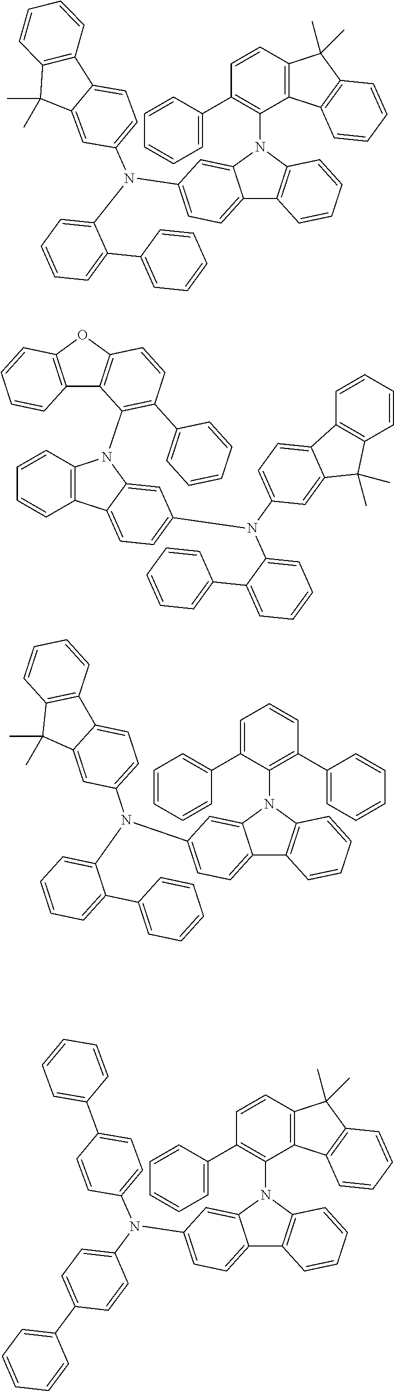 [SMILES: CC1(C)c2ccccc2-c2ccc(N(c3ccc4c5ccccc5n(-c5c(-c6ccccc6)ccc6c5-c5ccccc5C6(C)C)c4c3)c3ccccc3-c3ccccc3)cc21 ; CC1(C)c2ccccc2-c2ccc(N(c3ccc4c5ccccc5n(-c5c(-c6ccccc6)ccc6oc7ccccc7c56)c4c3)c3ccccc3-c3ccccc3)cc21 ; CC1(C)c2ccccc2-c2ccc(N(c3ccc4c5ccccc5n(-c5c(-c6ccccc6)cccc5-c5ccccc5)c4c3)c3ccccc3-c3ccccc3)cc21 ; CC1(C)c2ccccc2-c2c1ccc(-c1ccccc1)c2-n1c2ccccc2c2ccc(N(c3ccc(-c4ccccc4)cc3)c3ccc(-c4ccccc4)cc3)cc21]

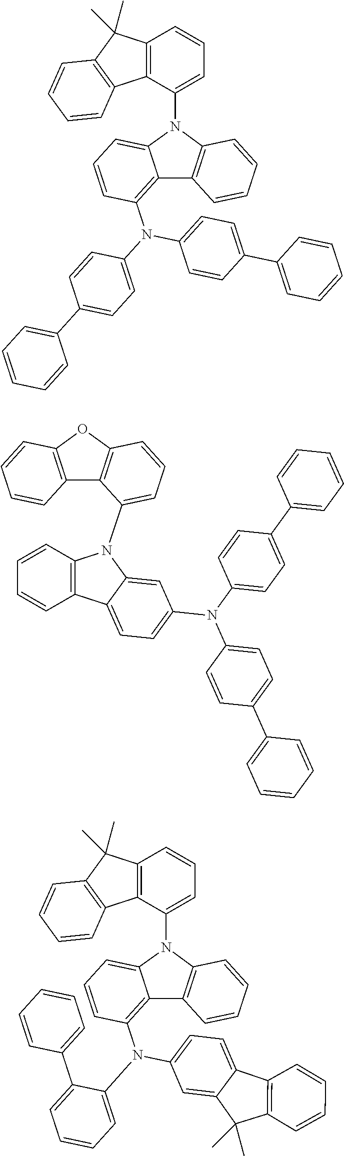 [SMILES: CC1(C)c2ccccc2-c2c(-n3c4ccccc4c4c(N(c5ccc(-c6ccccc6)cc5)c5ccc(-c6ccccc6)cc5)cccc43)cccc21 ; c1ccc(-c2ccc(N(c3ccc(-c4ccccc4)cc3)c3ccc4c5ccccc5n(-c5cccc6oc7ccccc7c56)c4c3)cc2)cc1 ; CC1(C)c2ccccc2-c2ccc(N(c3ccccc3-c3ccccc3)c3cccc4c3c3ccccc3n4-c3cccc4c3-c3ccccc3C4(C)C)cc21]

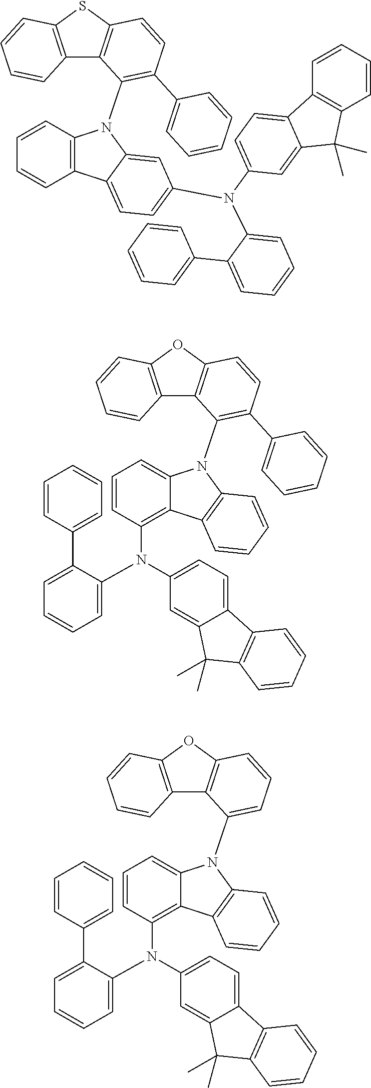 [SMILES: CC1(C)c2ccccc2-c2ccc(N(c3ccc4c5ccccc5n(-c5c(-c6ccccc6)ccc6sc7ccccc7c56)c4c3)c3ccccc3-c3ccccc3)cc21 ; CC1(C)c2ccccc2-c2ccc(N(c3ccccc3-c3ccccc3)c3cccc4c3c3ccccc3n4-c3c(-c4ccccc4)ccc4oc5ccccc5c34)cc21 ; CC1(C)c2ccccc2-c2ccc(N(c3ccccc3-c3ccccc3)c3cccc4c3c3ccccc3n4-c3cccc4oc5ccccc5c34)cc21]

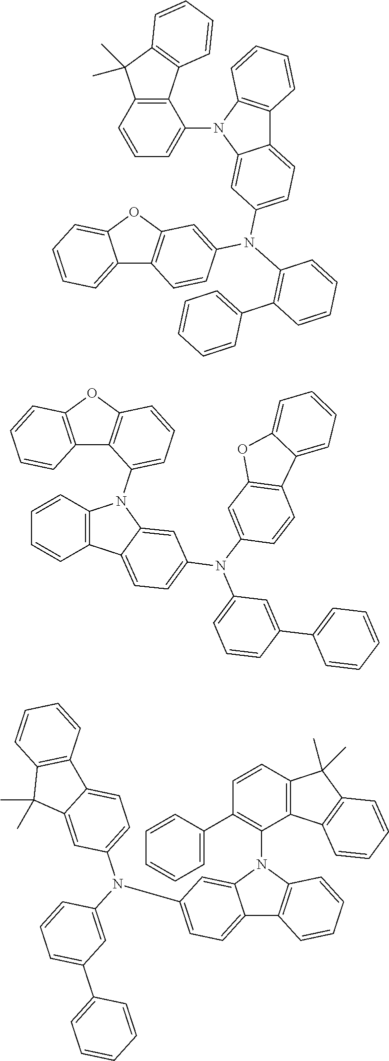

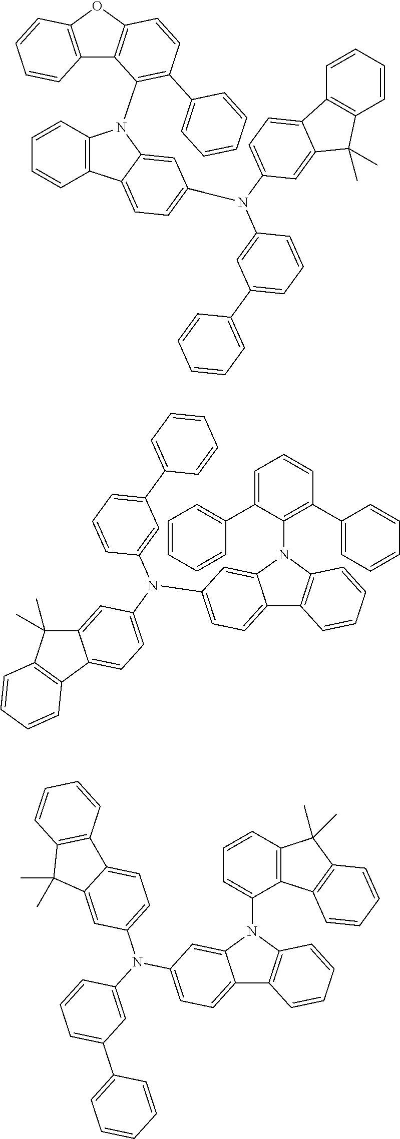 [SMILES: CC1(C)c2ccccc2-c2ccc(N(c3cccc(-c4ccccc4)c3)c3ccc4c5ccccc5n(-c5c(-c6ccccc6)ccc6oc7ccccc7c56)c4c3)cc21 ; CC1(C)c2ccccc2-c2ccc(N(c3cccc(-c4ccccc4)c3)c3ccc4c5ccccc5n(-c5c(-c6ccccc6)cccc5-c5ccccc5)c4c3)cc21 ; CC1(C)c2ccccc2-c2ccc(N(c3cccc(-c4ccccc4)c3)c3ccc4c5ccccc5n(-c5cccc6c5-c5ccccc5C6(C)C)c4c3)cc21]

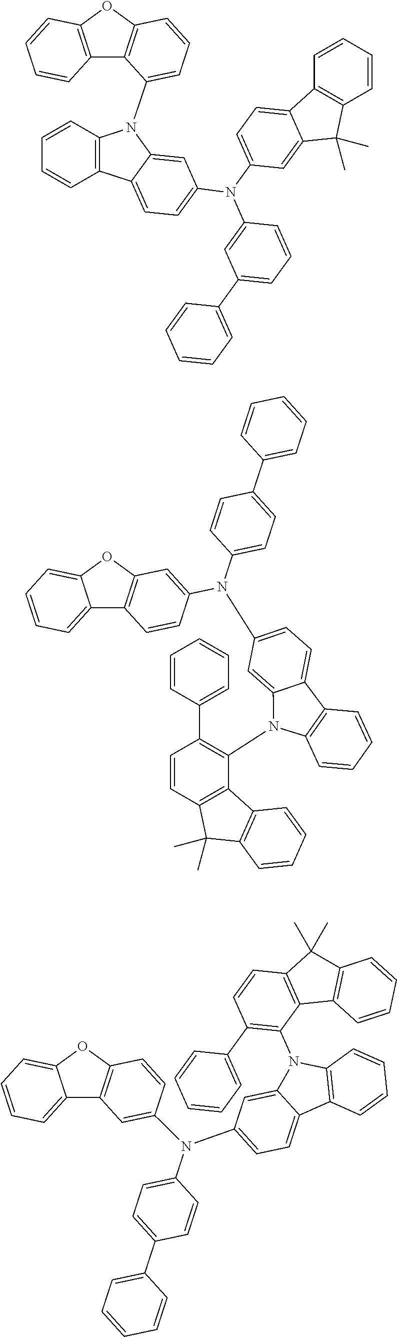 [SMILES: CC1(C)c2ccccc2-c2ccc(N(c3cccc(-c4ccccc4)c3)c3ccc4c5ccccc5n(-c5cccc6oc7ccccc7c56)c4c3)cc21 ; CC1(C)c2ccccc2-c2c1ccc(-c1ccccc1)c2-n1c2ccccc2c2ccc(N(c3ccc(-c4ccccc4)cc3)c3ccc4c(c3)oc3ccccc34)cc21 ; CC1(C)c2ccccc2-c2c1ccc(-c1ccccc1)c2-n1c2ccccc2c2ccc(N(c3ccc(-c4ccccc4)cc3)c3ccc4oc5ccccc5c4c3)cc21]

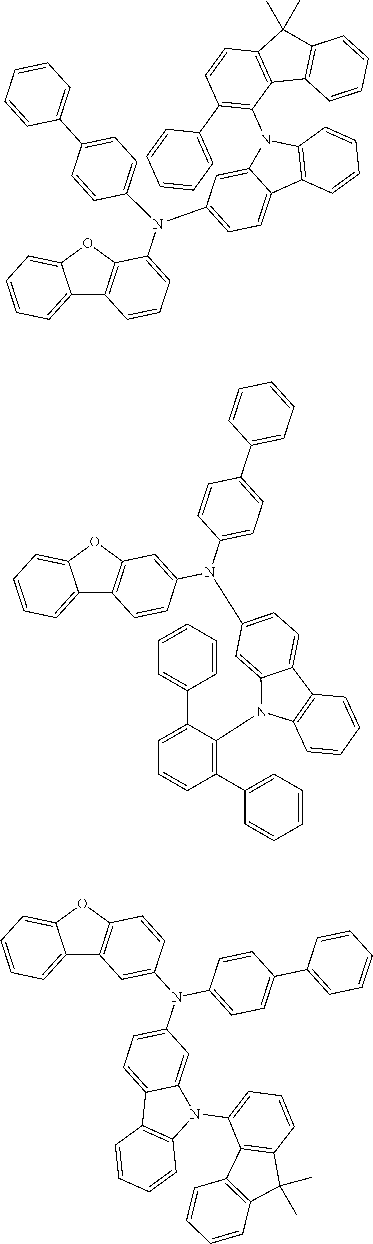 [SMILES: CC1(C)c2ccccc2-c2c1ccc(-c1ccccc1)c2-n1c2ccccc2c2ccc(N(c3ccc(-c4ccccc4)cc3)c3cccc4c3oc3ccccc34)cc21 ; c1ccc(-c2ccc(N(c3ccc4c(c3)oc3ccccc34)c3ccc4c5ccccc5n(-c5c(-c6ccccc6)cccc5-c5ccccc5)c4c3)cc2)cc1 ; CC1(C)c2ccccc2-c2c(-n3c4ccccc4c4ccc(N(c5ccc(-c6ccccc6)cc5)c5ccc6oc7ccccc7c6c5)cc43)cccc21]

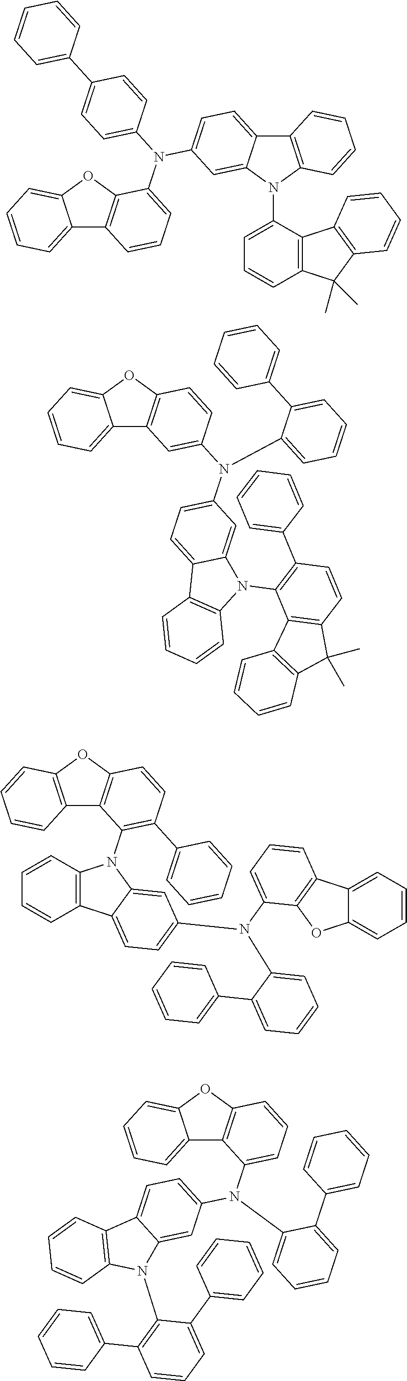

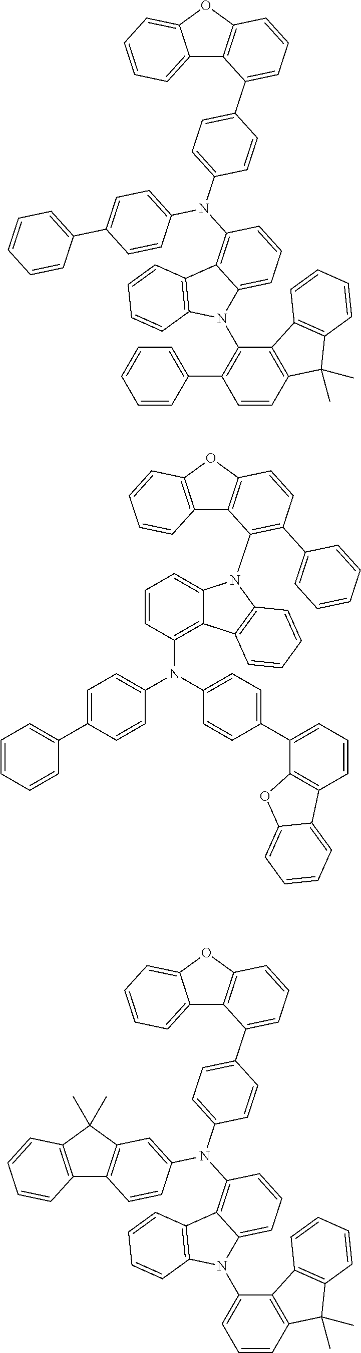 [SMILES: CC1(C)c2ccccc2-c2c1ccc(-c1ccccc1)c2-n1c2ccccc2c2c(N(c3ccc(-c4ccccc4)cc3)c3ccc(-c4cccc5oc6ccccc6c45)cc3)cccc21 ; c1ccc(-c2ccc(N(c3ccc(-c4cccc5c4oc4ccccc45)cc3)c3cccc4c3c3ccccc3n4-c3c(-c4ccccc4)ccc4oc5ccccc5c34)cc2)cc1 ; CC1(C)c2ccccc2-c2ccc(N(c3ccc(-c4cccc5oc6ccccc6c45)cc3)c3cccc4c3c3ccccc3n4-c3cccc4c3-c3ccccc3C4(C)C)cc21]

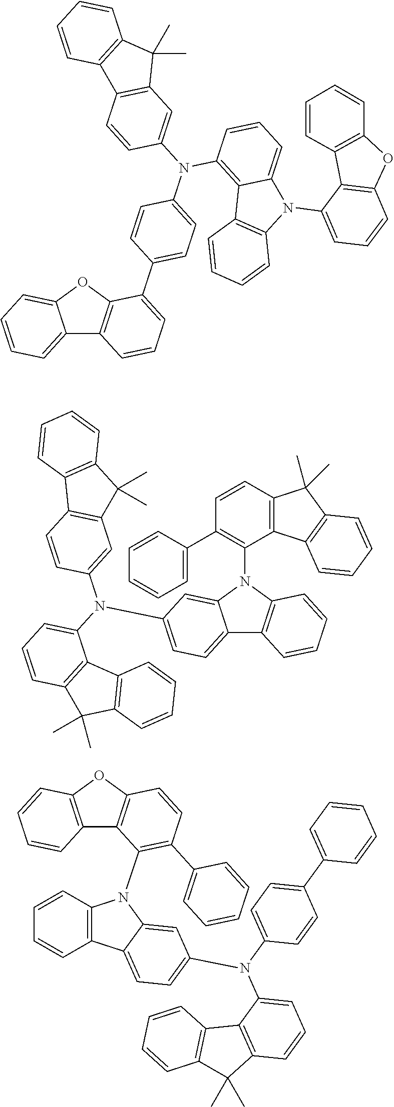

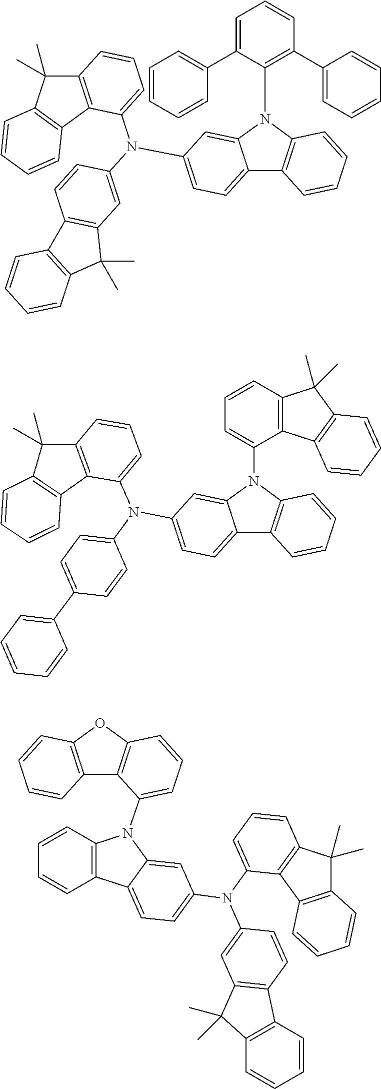 [SMILES: CC1(C)c2ccccc2-c2ccc(N(c3ccc4c5ccccc5n(-c5c(-c6ccccc6)cccc5-c5ccccc5)c4c3)c3cccc4c3-c3ccccc3C4(C)C)cc21 ; CC1(C)c2ccccc2-c2c(N(c3ccc(-c4ccccc4)cc3)c3ccc4c5ccccc5n(-c5cccc6c5-c5ccccc5C6(C)C)c4c3)cccc21 ; CC1(C)c2ccccc2-c2ccc(N(c3ccc4c5ccccc5n(-c5cccc6oc7ccccc7c56)c4c3)c3cccc4c3-c3ccccc3C4(C)C)cc21]

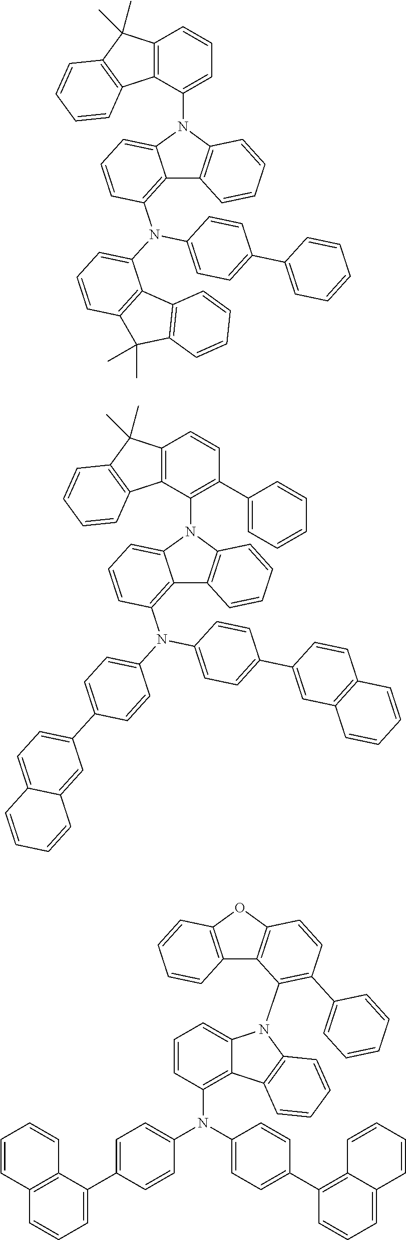

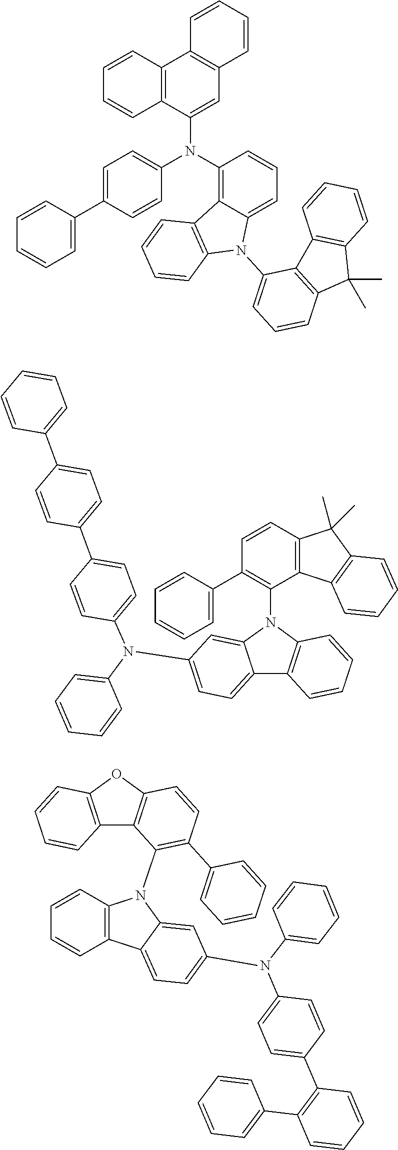

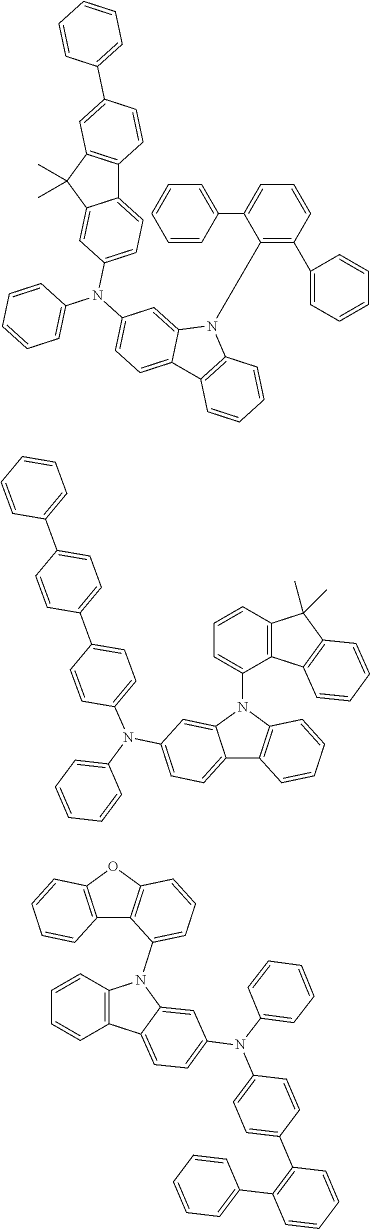

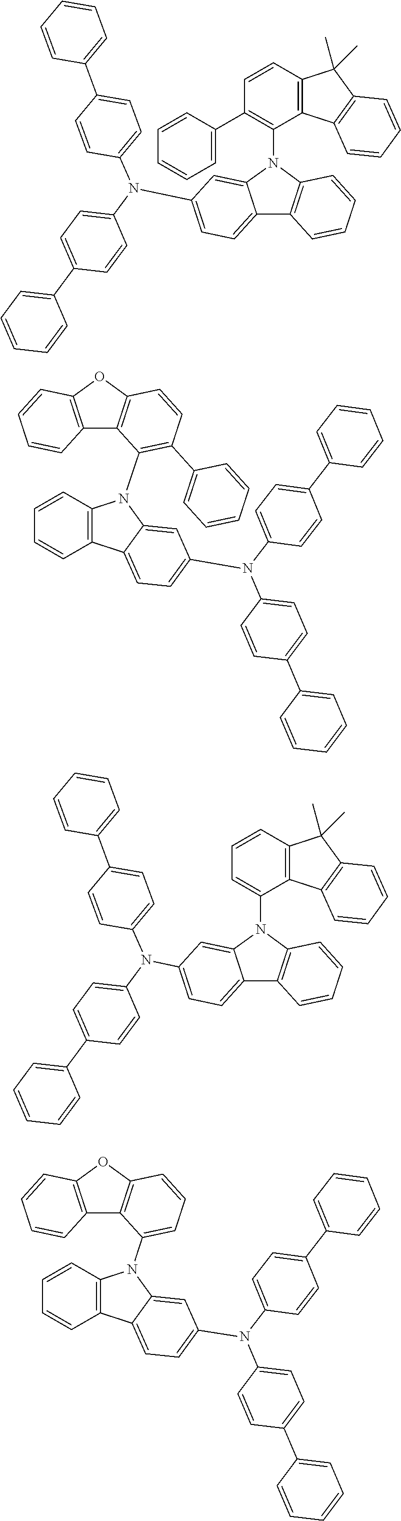

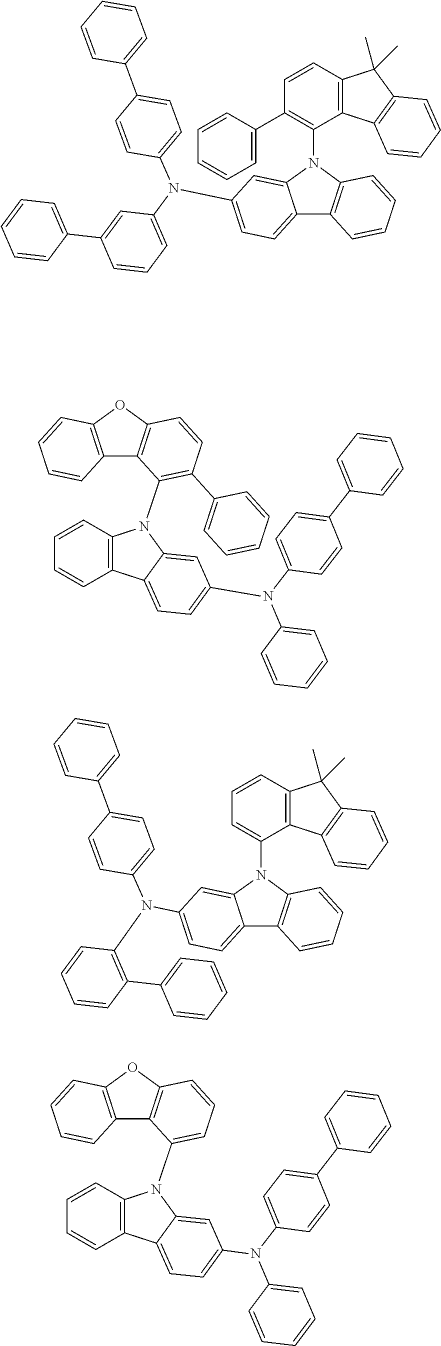 [SMILES: CC1(C)c2ccccc2-c2c1ccc(-c1ccccc1)c2-n1c2ccccc2c2ccc(N(c3ccc(-c4ccccc4)cc3)c3cccc(-c4ccccc4)c3)cc21 ; c1ccc(-c2ccc(N(c3ccccc3)c3ccc4c5ccccc5n(-c5c(-c6ccccc6)ccc6oc7ccccc7c56)c4c3)cc2)cc1 ; CC1(C)c2ccccc2-c2c(-n3c4ccccc4c4ccc(N(c5ccc(-c6ccccc6)cc5)c5ccccc5-c5ccccc5)cc43)cccc21 ; c1ccc(-c2ccc(N(c3ccccc3)c3ccc4c5ccccc5n(-c5cccc6oc7ccccc7c56)c4c3)cc2)cc1]

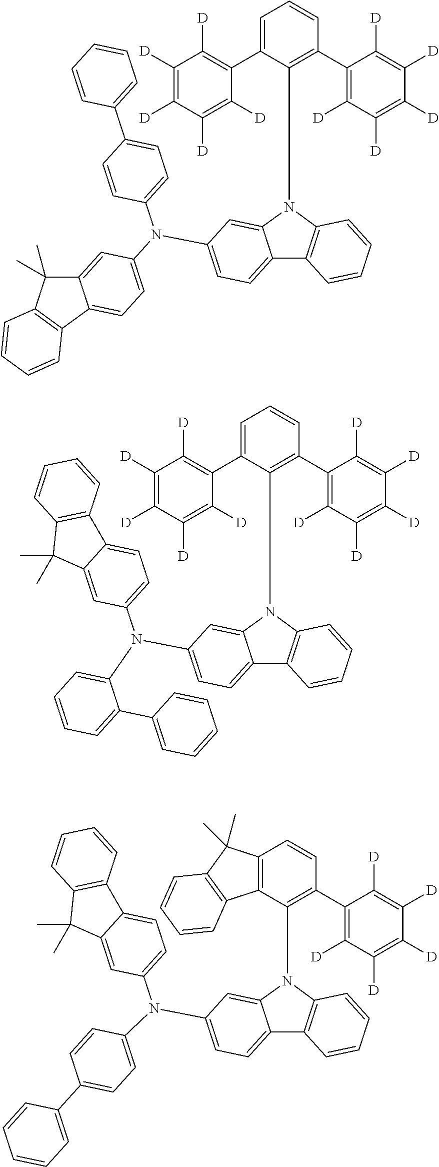 [SMILES: [2H]c1c([2H])c([2H])c(-c2cccc(-c3c([2H])c([2H])c([2H])c([2H])c3[2H])c2-n2c3ccccc3c3ccc(N(c4ccc(-c5ccccc5)cc4)c4ccc5c(c4)C(C)(C)c4ccccc4-5)cc32)c([2H])c1[2H] ; [2H]c1c([2H])c([2H])c(-c2cccc(-c3c([2H])c([2H])c([2H])c([2H])c3[2H])c2-n2c3ccccc3c3ccc(N(c4ccc5c(c4)C(C)(C)c4ccccc4-5)c4ccccc4-c4ccccc4)cc32)c([2H])c1[2H] ; [2H]c1c([2H])c([2H])c(-c2ccc3c(c2-n2c4ccccc4c4ccc(N(c5ccc(-c6ccccc6)cc5)c5ccc6c(c5)C(C)(C)c5ccccc5-6)cc42)-c2ccccc2C3(C)C)c([2H])c1[2H]]

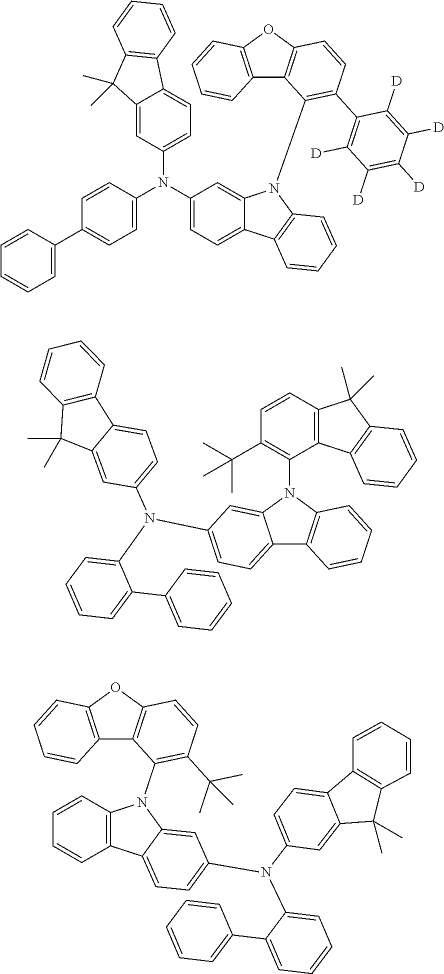 [SMILES: [2H]c1c([2H])c([2H])c(-c2ccc3oc4ccccc4c3c2-n2c3ccccc3c3ccc(N(c4ccc(-c5ccccc5)cc4)c4ccc5c(c4)C(C)(C)c4ccccc4-5)cc32)c([2H])c1[2H] ; CC(C)(C)c1ccc2c(c1-n1c3ccccc3c3ccc(N(c4ccc5c(c4)C(C)(C)c4ccccc4-5)c4ccccc4-c4ccccc4)cc31)-c1ccccc1C2(C)C ; CC(C)(C)c1ccc2oc3ccccc3c2c1-n1c2ccccc2c2ccc(N(c3ccc4c(c3)C(C)(C)c3ccccc3-4)c3ccccc3-c3ccccc3)cc21]

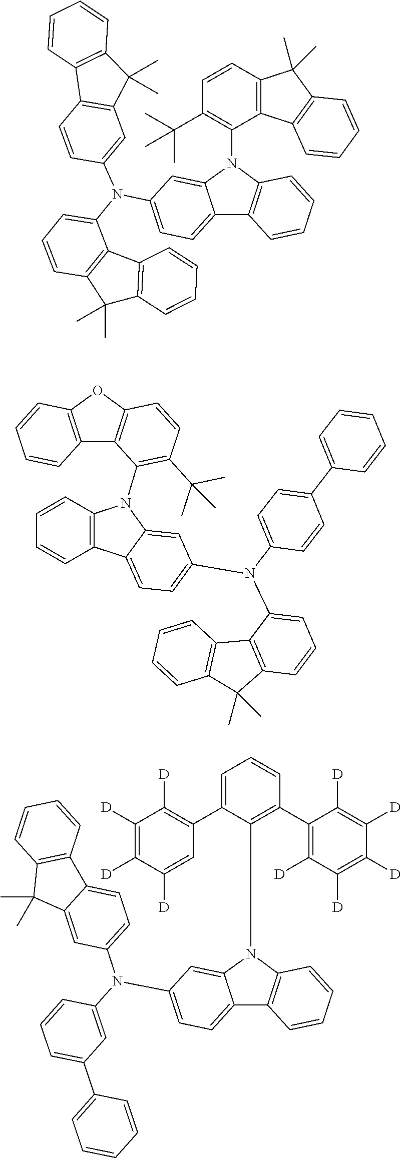

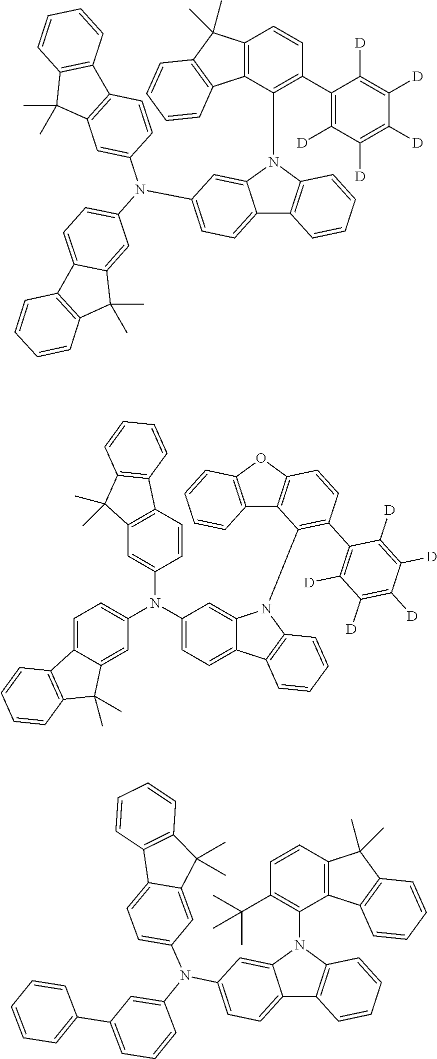 [SMILES: [2H]c1c([2H])c([2H])c(-c2ccc3c(c2-n2c4ccccc4c4ccc(N(c5ccc6c(c5)C(C)(C)c5ccccc5-6)c5ccc6c(c5)C(C)(C)c5ccccc5-6)cc42)-c2ccccc2C3(C)C)c([2H])c1[2H] ; [2H]c1c([2H])c([2H])c(-c2ccc3oc4ccccc4c3c2-n2c3ccccc3c3ccc(N(c4ccc5c(c4)C(C)(C)c4ccccc4-5)c4ccc5c(c4)C(C)(C)c4ccccc4-5)cc32)c([2H])c1[2H] ; CC(C)(C)c1ccc2c(c1-n1c3ccccc3c3ccc(N(c4cccc(-c5ccccc5)c4)c4ccc5c(c4)C(C)(C)c4ccccc4-5)cc31)-c1ccccc1C2(C)C]

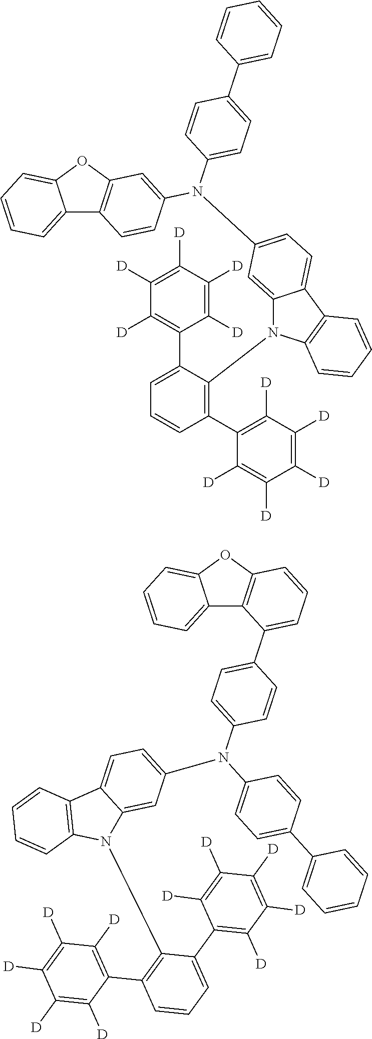 [SMILES: [2H]c1c([2H])c([2H])c(-c2cccc(-c3c([2H])c([2H])c([2H])c([2H])c3[2H])c2-n2c3ccccc3c3ccc(N(c4ccc(-c5ccccc5)cc4)c4ccc5c(c4)oc4ccccc45)cc32)c([2H])c1[2H] ; [2H]c1c([2H])c([2H])c(-c2cccc(-c3c([2H])c([2H])c([2H])c([2H])c3[2H])c2-n2c3ccccc3c3ccc(N(c4ccc(-c5ccccc5)cc4)c4ccc(-c5cccc6oc7ccccc7c56)cc4)cc32)c([2H])c1[2H]]

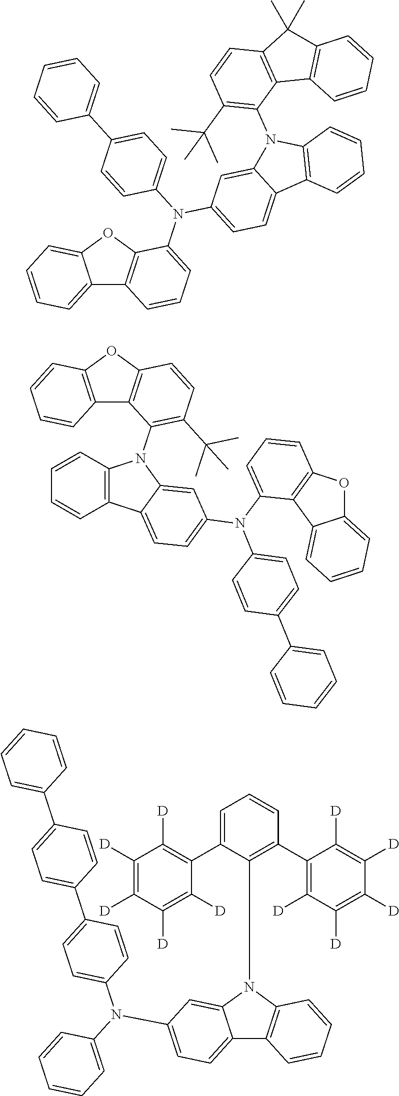 [SMILES: CC(C)(C)c1ccc2c(c1-n1c3ccccc3c3ccc(N(c4ccc(-c5ccccc5)cc4)c4cccc5c4oc4ccccc45)cc31)-c1ccccc1C2(C)C ; CC(C)(C)c1ccc2oc3ccccc3c2c1-n1c2ccccc2c2ccc(N(c3ccc(-c4ccccc4)cc3)c3cccc4oc5ccccc5c34)cc21 ; [2H]c1c([2H])c([2H])c(-c2cccc(-c3c([2H])c([2H])c([2H])c([2H])c3[2H])c2-n2c3ccccc3c3ccc(N(c4ccccc4)c4ccc(-c5ccc(-c6ccccc6)cc5)cc4)cc32)c([2H])c1[2H]]

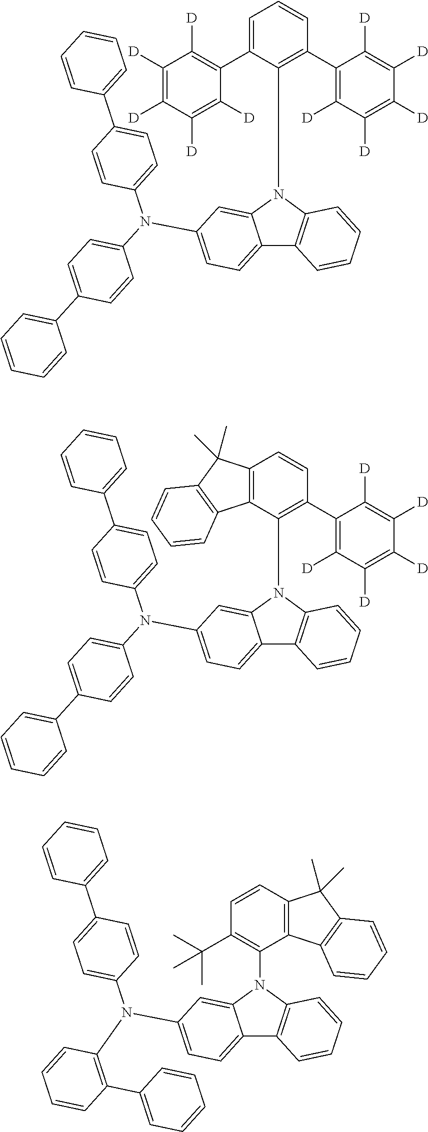 [SMILES: [2H]c1c([2H])c([2H])c(-c2cccc(-c3c([2H])c([2H])c([2H])c([2H])c3[2H])c2-n2c3ccccc3c3ccc(N(c4ccc(-c5ccccc5)cc4)c4ccc(-c5ccccc5)cc4)cc32)c([2H])c1[2H] ; [2H]c1c([2H])c([2H])c(-c2ccc3c(c2-n2c4ccccc4c4ccc(N(c5ccc(-c6ccccc6)cc5)c5ccc(-c6ccccc6)cc5)cc42)-c2ccccc2C3(C)C)c([2H])c1[2H] ; CC(C)(C)c1ccc2c(c1-n1c3ccccc3c3ccc(N(c4ccc(-c5ccccc5)cc4)c4ccccc4-c4ccccc4)cc31)-c1ccccc1C2(C)C]

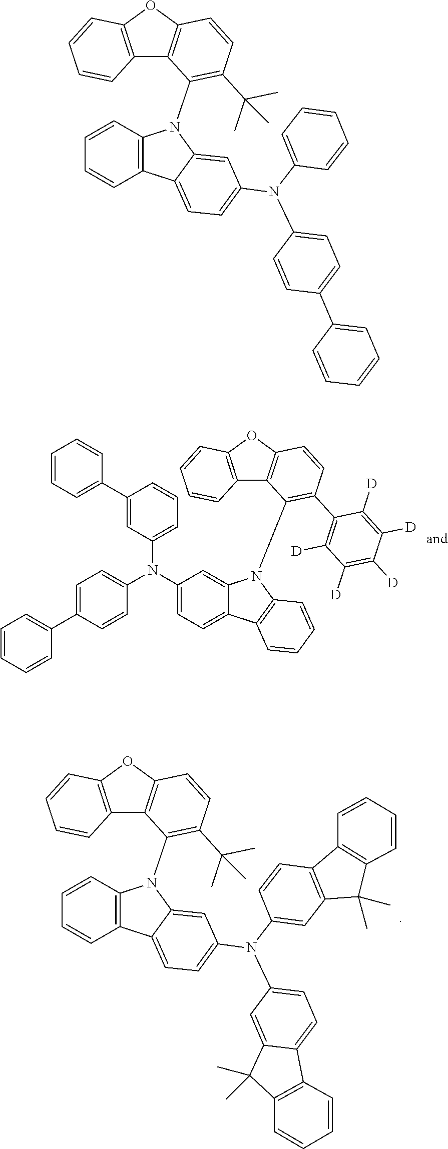 [SMILES: CC(C)(C)c1ccc2oc3ccccc3c2c1-n1c2ccccc2c2ccc(N(c3ccccc3)c3ccc(-c4ccccc4)cc3)cc21 ; [2H]c1c([2H])c([2H])c(-c2ccc3oc4ccccc4c3c2-n2c3ccccc3c3ccc(N(c4ccc(-c5ccccc5)cc4)c4cccc(-c5ccccc5)c4)cc32)c([2H])c1[2H] ; CC(C)(C)c1ccc2oc3ccccc3c2c1-n1c2ccccc2c2ccc(N(c3ccc4c(c3)C(C)(C)c3ccccc3-4)c3ccc4c(c3)C(C)(C)c3ccccc3-4)cc21]

7. An application of the compound containing the carbazole derivative according to claim 1 in the preparation of an organic electroluminescent device.

8. An organic electroluminescent device, characterized in that the organic electroluminescent device comprises one or more compounds containing carbazole derivatives according to claim 1.

9. The organic electroluminescent device according to claim 8, characterized in that the organic electroluminescent device comprises an anode, a hole transport layer, a luminescent auxiliary layer, a luminescent layer, an electron transport layer, an electron injection layer, and a cathode, which are sequentially arranged on the substrate, wherein the hole transport layer comprises one or more compounds containing carbazole derivatives according to claim 1.
